(19) United States
(12) Patent Application Publication
(10) Pub. No.: US 2024/0385137 A1
(43) Pub. Date: Nov. 21, 2024

(54) Solid State Multiple-functioning Nanostructured Organometallic D-Wave Pi Josephson Junction Toroidal Arrays Superconductive Quantum Interference Devices (SQUID) of Making and Applications Thereto

(71) Applicant: Ellen Tuanying Chen, Rockville, MD (US)

(72) Inventor: Ellen Tuanying Chen, Rockville, MD (US)

(21) Appl. No.: 18/199,845

(22) Filed: May 19, 2023

(51) Int. Cl. G01N 27/327; C12Q 1/00; C12Q 1/37; G01N 27/30; G01N 27/447

(52) U.S. Cl. CPC G01N 27/3273; C12Q 1/005; C12Q 1/37; G01N 27/308; G01N 27/44791

(57) ABSTRACT

The present invention provides a multiple functioning system for quantum sensing, scalable long time energy storage and quantum computing, as well as providing a dielectric insulator coating method, a solid-state component approach, and a delocalized direct-electron transfer relay in the nanopores “Five-petal Starflower”-like structured membrane to overcome the d-wave Pi JJ ground energy instability, and enables to act as a gate-controlled diode bridge suppressing supercurrent at a low scan rate. The system comprises multiple-layer organo-metallic cross-linked polymers forming various superlattice nanostructured “Five-petal Starflower”-like toroidal nano array membranes based on for sensing Cooper-pair wave transmissions causing intrinsic magnetic flux quantum observed based on a Josephson junction toroidal array, an insulator coating material and method, and a long JJ tunneling in the membranes promoted Cooper-pairs cross the Josephson toroidal junction barriers at zero-bias, and magnifying the quantum conductance. The One-Device-Assembly system enables a pj energy consumption for quantum qubits; or acting as a long-time energy storage device with high energy density; also acted as a quantum sensor sensing the presence of sub-nanogram per mL to 50 μg/mL level collagen that is inversely proportional to the supercurrent at zero bias under a media-free and electrolyte-free condition at room-temperature without an external magnetic field applied.

### CROSS-REFERENCE TO RELATED APPLICATIONS

This patent application entitled of “Solid State Nanostructured Organometallic D-Wave Pi Josephson Junction Toroidal Arrays Superconductive Quantum Interference Devices (SQUID) of Making and Applications Thereto” is a Continuation in Part of US patent application that claims the benefit of the U.S. Non-Provisional patent application Ser. No. 16/742,745 in titled of “Nanostructured Biomimetic Protein Superconductive Devices of Making and Its Multiple Applications Thereto” filed on Jan. 14, 2020. The entire disclosure of the prior Patent Application Ser. No. 62/848,422 in the title Nanostructured Biomimetic Organo-metallic Superconductive Devices of Making and its applications for Direct Real-time Monitoring Atto-Molarity ATP in Biological Specimens” filed on May 15, 2019, is hereby incorporated by reference, as is set forth herein in its entirety.

### FIELD OF THE INVENTION

The present invention relates to the field of applications in the superconductor, in particular, to a device having both characteristics in superconductivity and memristive/memcapacitive/meminductive embedded with non-ferromagnetic switches functioning at room temperature and its applications in sensing biological specimens.

The present CIP invention relates to the field of room temperature superconductors, in particular, to a system having multiple characteristics in superconducting, mems-ristor/mems-capacitor/mems-inductor, quantum sensing, quantum computing, and long-time energy storage in all solid state.

### BACKGROUND OF THE INVENTION

Real-time monitoring of ATP in biological specimens is a convenient means to monitor hygiene in healthcare units and for medical devices. Rapid and precise monitoring has been in strong demand from the public and under tight regulations [1-8].

Researchers found a rapid measurement of ATP in a series of diluted bacterial cultures correlates with the bacterial concentrations contaminated in the biological specimens, thus ATP testing becomes an important indicator for monitoring hygiene in healthcare units [9-11].

The predominate luciferase bioluminescent sensor method, the fluorescence in situ hybridization (FISH) method, the HPLC method, the flow cytometry method, and the immobilized gene electrochemical sensor method are common methods for ATP [7-14]. Among these methods, the luciferase bioluminescent sensor method has been recommended by CDC for assessing hospital device surface hygiene and has been used for decades. Nante's review article revealed the ATP luciferase bioluminescent portable sensor method is not a standardized methodology, because each tool has different benchmark values, not always clearly defined. The authors stressed this technique could be used to assess, in real-time, hospital surfaces' cleanliness, but has its limitations of not accurate in detecting bacteria, and the requirement of the washing out of the disinfectant step on the surfaces before testing are draw backs [14]. The gene method could improve the detection limits to 10 fM ATP, but it is not real-time monitoring and the procedures are burdensome [12]. Even NASA recently recognized an unmet need for real-time online assessing the drinking water contamination for astronauts who are on their space flight, because all current methods used for monitoring water quality including the ATP bioluminescent and gene methods were deemed unfit because the requirements of (1) rapid accurate reagent-free real-time testing; (2) the testing machine needs to be lightweight and small in size; (3) there should be no sample preparation steps, have not met [15]. In the face of the demand and challenges, our group proposed an innovative approach to attack the problems, that is to develop superlattice nanostructured superconductive/memristive sensors having organo-metallic crossed-linking polymer membranes which work at Josephson Junction at the zero-bias potential, may overcome protein nonspecific bounding and increase accuracy, based upon our prior experience using the superconductive/memristive device to enable direct detection of collagen-1 in 16.7 atto molarities with higher than 96% accuracy using fresh human capillary blood serum specimens at low (8.3 fM) and high (0.55 nM) levels having imprecision of 4.9% and 0.8%, respectively compared with the controls, under antibody-frec, tracer-free, and reagent-free conditions at room temperature [16-17].

Recent theoretical predictions of Josephson-based meminductive and memristive quantum superconducting devices not only have multiple state superposition properties but also the Cooper-pair waves behave hysterically have drawn attention [18-20]. Herein by utilizing these properties, we assert that significantly improving the ATP testing method may be accomplishable. We planned to develop two types of sensors, namely, the biomimetic matrix metalloproteinase (MMP-2) Sensor 1 at its active state by a heating method to switch “Off” the cysteine group in the membrane, which is ready for biocommunication with ATP; then the results due to ATP's interaction will be compared with Sensor 2. also at its active state of the biomimetic MMP-2, but by a direct fabrication method of organo-metallic self-assembled cross-linked polymer without cysteine. It is well-known that superlattice membranes have been used as candidates for applications in superconductivity [21].

Literature reported ATP hydrolyzation extracellularly induces cancer cells' drug resistance [22-25]. Assessing human milk's immunological advantage over cow milk in preventing extracellular ATP hydrolyzation is important to human health. Shonhai's group published a review article regarding the roles of the Heat Shock Proteins (HSP) that acted as immunomodulates whose capability is to transform the anti-inflammatory property when the HSP concentrations are low to the pro-inflammatory property when HSP concentrations are high [26], and unfortunately, the authors did not define the concentration range. We felt an unmet need exists for this health-related important topic. Extracellular ATP concentration and intracellular ATP concentration ranges are important related to HSPs' functions either in physiological or pathological function, because HSPs primarily occur

extracellularly, but also reported from literature, HSPs occur in an intracellular micro-environment [27]. If we can build a well-characterized and well-controlled system to define the critical HSP concentration ranges in the transformation between the two statuses that would be a primary attempt toward a resolution.

### Following are the Background of the Invention of the CIP

Pi Josephson junction of D-wave materials made Superconductive-insulator-superconductive (S-I-S) Josephson junction circuit element increased interest for the applications of a phase spin splitter and alternation of magnetism [1-5]. A Pi Josephson junction is a Josephson junction in which the Josephson phase o equals x in the ground state, i.e. when no external current or magnetic field applied, however, the corresponding Josephson energy is unstable, because it reached the maximum [6]. D-wave superconductive toroidal Josephson junction array devices made by metalloorganic superconductive polymers-insulator-mem-element (S-I-M) organic polymers demonstrated many applications, such as quantum sensing, energy storage, and quantum entanglement computing at room-temperature under reagent-free and external magnetic field-free conditions studied at our group [7-10]. Recently our group published an article using a d-wave mixed-spin triplet state Cooper-pair to penetrate toroidal

Josephson junction barriers and for the applications of increased energy storage in a methanol media [11]. A key manufacturing process for batteries has to consume tons of toxic organic solvents per day, herein it is crucial to reduce the amount of organic solvent usage. The goals of the present research project are to provide a new generation superconductive Josephson toroidal array of pi junctions in all solid states under media-free and external magnetic field-free conditions working at room temperature for overcoming pi junction energy instability at the ground state. providing this device as a platform of extended energy long-time storage. Further goal of this project is to find an innovative application in room temperature Josephson diode with a higher diode effecincy.

Our approaches to reach the goals are 1), fabricate two types of Josephson junctions: one is the JJ junction using known superconductive double-layer metal-organic polymer materials coated on the surface of a glassy carbon (GC) electrode as(S), separated by a collagen-coated dielectric insulator as (I), and another GC electrode having known functions of memristive/memcapacitive organic conductive polymer coated onto the electrode surface as (Mem) assembling together as a SIM configuration; the collagen-coated insulator serves as the primary JJ; the second types JJ is based on the zinc ion formed superlattice by chelating with functional groups of the superconductive double-layer metal-organic polymers as an S-I-S configuration, here the zinc ions served as insulators. 2), build a long Josephson junction (LJJ) by promoting Cooper-pairs long tunneling through the delocalizing direct-electron transfer (DET) forming relaying chain under media-free and electrolyte-free conditions. This is our hypothesis that if one of our established platforms in room-temperature superconducting devices were based on Cooper-pairs DET effect under using a media (either in PBS or in methanol) and an analyte of collagen to extend the DET from local DET to a long range DET relay, then we would be able to take away the media, emphasize the effect of collagen coating on an insulator, it may promote Cooper-pair's LJJ and overcome the d-wave Pi Josephson junction ground state energy unstable problem. Wherein our goal for all solid components, media-free, and electrolyte-free can be accomplished, as the benefits were the long-time energy storage with stability [1, 2, 12-15].

### SUMMARY OF THE INVENTION

It is an object of the present invention to provide a new generation of organo-metallic superconductive, memristive/memcapacitive device compromising of the active sites of a biomimetic Matrix Metalloproteinase (MMP-2) in the membrane of the device.

It is an object of the present invention to provide a new generation of organo-metallic superconductor/memristor/memcapacitor with multiple-layer curvature structure mimicking the function and structure of cation-diffusive facilitator (CDF) protein YiiP of E. coli (gram-negative), and to efflux zinc ions from cellular membrane.

It is an object of the present invention to further provide the biomimetic YiiP protein which is capable to be a periplasmic zinc chaperone for providing zinc to E. coli when it is in a starvation zinc state.

It is a further object of the present invention to provide the biomimetic YiiP protein which is capable to be a Heat Shock Protein (HSP60) chaperone of GroEL in helping folding of a protein in its cavity.

It is a further object of the present invention to provide the biomimetic MMP-2 sensor which is able to sense aM ATP concentration presence in biological samples in a direct, rapid real-time monitoring fashion of hospital instrumental hygiene without using an antibody, labeling, and other burdensome procedures.

It is a further object of the present invention to provide the biomimetic MMP-2 sensor which is able to be memory storage and a superlattice quantum computing chip working at room temperature.

It is a further object of the present invention to provide a method that can be optimally operated for fabrication of the self-assembled 3D-nanocage structure multiple-layer membrane on an electrode surface acted as a living biological cell model to assess the immunomodulant concentration effect.

It is a further object of the present invention to provide a device having multiple utilities, not only in sensing multiple analytes, but also can be used for defining the immunomodulant concentration effect on transformations from an anti-inflammatory to a pro-inflammatory status in the presence of LPS challenge with a wide range ATP concentration change in the biological specimens.

It is a further object of the present invention to provide a device to direct monitor the reversible membrane potential change under the influence of ATP concentrations.

### Following are the Summaries of the Instant Invention for the CIP Application

It is an object of the instant invention to provide a new generation of room-temperature organometallic superconductive and memristive/memcapacitive devices working in all solid components under media-free and electrolyte-free conditions.

It is an object of the instant invention to provide a new generation of room-temperature organometallic superconductive and memristive/memcapacitive devices comprising of flexible Josephson toroidal junction array that has the capability of stabling D-wave Pi junction ground energy.

It is an object of the instant invention to provide a new generation of room-temperature organometallic superconductive and memristive/memcapacitive devices comprising of a dielectric insulator with a layer of coating material, which the coating process is free from using organic toxic media, and the coated dielectric insulator serves with a function for the “Primary Josephson Junction” barrier.

It is an object of the instant invention to provide a new generation of room-temperature organometallic superconductive and memristive/memcapacitive devices comprising of superconductive layer comprises of conductive organic cross-linked polymers and metal ions, which the metal ions serve of “secondary Josephson junction” barrier.

It is an object of the instant invention to provide a new generation of room-temperature organometallic superconductive and memristive/memcapacitive devices having a delocalized long Cooper-pair tunneling that forms d-wave pi junction coherently between the primary and the secondary JJ toroidal arrays coherently based on the direct-electron transfer (DET) relay enabling across multiple barriers taken advantage of anharmonic Josephson Oscillation.

It is a further object of the instant invention to provide a new generation of room-temperature organometallic superconductive and memristive/memcapacitive device that operates in multiple functions, for quantum computing, quantum sensing, and long-time stable energy storage.

It is a further object of the instant invention to provide a new generation of room-temperature organometallic superconductive and memristive/memcapacitive device that operates under free of external magnetic fields applied.

It is a further object of the instant invention to provide a new generation of room-temperature gate-controlled diode bridge forming SIS Josephson junction array that can control supercurrent leading to superconducting logics like CMOS logics having low energy dissipation.

### BRIEF DESCRIPTION OF THE DRAWINGS

FIG. 1A depicts the 2D AFM membrane image in the native state of the biomimetic MMP-2 device. FIG. 1B depicts the cross-section analysis of the AFM membrane image.

FIG. 2A depicts the 3D AFM image of the activated biomimetic MMP-2 Sensor 1 by the heating method. FIG. 2B depicts the enlarged 2D AFM image with the same z-value range between 33.2 to −21.4 nm as the native state device in FIG. 1A for comparison.

FIG. 3A depicts the 2D image of the multiple-layer ring structure of the activated biomimetic MMP-2 Sensor 2 by the direct fabrication method in 0.734×0.734 μm². The ring structure parameters labeled as “1” are listed. FIG. 3B depicts the cross-section analysis of the AFM membrane image. FIG. 3C depicts the 3D AFM image from the bird's view. FIG. 3D depicts the enlarged image in 0.9×0.9 μm² shown in the flat area in FIG. 3I. FIG. 3E depicts the alternative band deep-band flat formation of the membrane which promotes the Cooper-pair electron cloud mobility in 1.6×1.6 μm². FIG. 3F depicts the enlarged 2D AFM image at high sensor mode in a top view of the biomimetic Heat Shock Protein (HSP) 60 chaperone structure having 7 subunits as shown on the top. The arrow indicates the mobile zinc ions. FIG. 3G depicts the top view in an amplitude mode of the AFM image of the large single porous biomimetic HSP60 structure. FIG. 3H depicts the side view of the 3D AFM image in high sensor mode for the single porous structure in 3×3.6 μm². FIG. 3I depicts the 3D AFM image of the tall cylinder structure of the biomimetic HSP60 in 10×10 μm².

FIG. 4 illustrates the scheme of the steps of direct detection of ATP using the 3D-nanocage structure biomimetic MMP-2 membrane sensor/superconductor approach.

Step 1: The Direct Electron-relay (DER) model of the 3D-nanocage polymer network structure between the zinc ions from the activated biomimetic MMP-2. by elimination of the cysteine group from the polymer mixture of triacetyl-β-cyclodextrin (TCD), polyethylene glycol diglycidyl ether (PEG), poly (4-vinyl pyridine) (PVP), bis-substituted imidazole dimethyl-β-cyclodextrin (bM-β-DMCD), cysteine and embedded zinc chloride in appropriate proportions_before deposited on the gold chip, and the mM-β-DMCD (short names as MCD) in Tris/HCl buffer. Step 2: Form a long-range DET in the presence of Tris/MCD media with ATP being included in the cyclodextrin cavities, that not only stabilized ATP but also invited ATP to participate in the long-range direct electron-relay chain between the media and the 3D-nanocage membrane electrode assembling (MEA); Step 3: the ATP interacts with the cage polymer network molecules forming enhanced DER without a need for an external power supply.

FIG. 5A depicts activated Sensor 1 by the heating method's i-V curves in different scan rates from 1 Hz to 25 kHz in pH 7.8 buffer (20 mM Tris/HCl buffer with 3 mM KCl, 130 mM NaCl and 0.5 mg/mL MCD), in short, as the Tris/HCl/MCD buffer. FIG. 5B depicts Sensor I's i-V curves under the influence of ATP concentrations over 40 fM to 60 nM compared with the control under the scan rate of 60 Hz in the Tris media.

FIG. 6 depicts the scan rate impacts on the i-V curves of the activated state biomimetic MMP-2 Sensor 2, by the direct fabrication method, over scan rate from 1 Hz to 25 kHz in the tris/HCl/MCD media.

FIG. 7 depicts Sensor 2's i-V curves transformation between memristive to superconductive at zero-bias potential under the influence of ATP from 25 aM to 2 mM compared with the control with a 60 Hz scan rate in the tris/HCl/MCD media. Panel A is the i-V curve for ATP 25 aM compared with the control; Panel B is for the i-V curve having 400 aM; Panel C: is ATP 400 fM; Panel D: is ATP 200 pM; Panel E: ATP 200 nM; Panel F: ATP 2 mM.

FIG. 8 depicts the comparison of the superconducting i-V curves at the zero-bias potential for Sensor 2 under the influence of ATP from 200 fM to 200 mM compared with the control under a 10 kHz scan rate in the tris/HCl/MCD media. Panel A: is the control; Panel B: is ATP 200 fM; Panel C: is ATP 400 fM; Panel D: is ATP 200 pM; Panel E: is ATP 200 nM; Panel F: is ATP 400 nM; Panel G: ATP 800 nM; Panel H: ATP 2.0 mM, and Panel I: ATP 200 mM in the media.

FIG. 9 depicts the supercurrent vs. ATP concentration curves of the DET_{red} and DETₒₓ peaks at the zero-bias potential with a 60 Hz scan rate for Sensor 2. The ATP concentration ranges from 400 aM to 2 mM. Data obtained according to Figures from FIG. 7B to 7F.

FIG. 10 depicts the supercurrent vs. ATP concentration curves of the DET_{red} and DETₒₓ peaks at the zero-bias potential at a 10 KHz scan rate for Sensor 2. The ATP concentration ranges from 200 fM to 200 mM. Data was obtained according to FIGS. 8A to 8I.

FIG. 11 depicts the impact of ATP concentrations at the Josephson junction on the supercurrent and the impact on the phase change of the waves at the zero-bias potential during 10 consecutive scans at 60Hz over the ATP concentrations from 400 fM (Panel A), 200 pM (Panel B), 200 nM (Paneol C), and to 2.0 mM (Panel D) in the Tris/HCl/MCD media.

FIG. 12A depicts 25 aM low concentration ATP in consecutive 5 scan cycles of san rate 60 Hz i-V curves compared with the control of Sensor 2. There was no phase change occurring and keeping the hysteresis curves. FIG. 12B depicts 400 fM concentrations ATP in consecutive 9 scan cycles of san rate 60 Hz i-V curves compared with the control, having a phase change occur and superconducting at the zero-bias potential. FIG. 12C also depicts the presence of 200 nM ATP concentrations, Sensor 2 shows the phase change and superconducting current at a 60 Hz scan rate.

FIG. 13 depicts the 3D dynamic map of the relationships over 5-level ATP concentrations from 0.2 pM to 2.0 mM over the potential range between −40 mV to +40 mV for illustration of the relationship among ATP concentrations, zero-bias potential, and the differential quantum conductance using 10 kHz scan rate forwarding scan data.

FIG. 14 depicts Sensor 1's real-time direct monitoring of current vs, time profiles over ATP concentrations over 100 aM to 400 nM (9 levels from curve a to k) vs. controls in the buffer solution. Samples run triplicates.

FIG. 15 depicts the lower-end concentration curves from a (the control) to e. Inserts are enlarged views for low-end concentration compared with controls.

FIG. 16 Panel A depicts the calibration curve of current density after subtracting the background current vs. ATP over 100 aM to 170 nM. FIG. 16 Panel B depicts the calibration curve over the linear range from 400 fM to 170 nM. Each sample run triplicates.

FIG. 17 depicts Sensor 2's open circuit potential curves for monitoring the energy change over 25 aM-400 pM ATP.

FIG. 18 depicts the calibration curve of voltage vs. ATP concentrations over 25 aM-400 pM.

FIG. 19 depicts Sensor 2's open circuit potential curves at the high-end ATP concentration over 0.8 nM to 2.0 mM.

FIG. 20 depicts the double log plot of the calibration curve of the open circuit potential vs. ATP concentrations from 0.8 nM to 2.0 mM.

FIG. 21 depicts Sensor 2's voltage vs. time curves by the Double-step Chronopotentialmetry (DSCPO) method in the presence of various ATP concentrations from 25 aM to 400 nM in the Tris/HCl/MCD buffer compared with the control. Each sample run triplicates.

FIG. 22A depicts the calibration curve of the normalized action potential divided by the mean signal vs. ATP concentrations over 100 aM to 200 nM. FIG. 22B depicts the low-end ATP calibration curve from ATP 25 aM to 400 aM in the Action potential of the DER peak.

FIG. 22C depicts the semi-log plot of the absolute normalized resting potential vs. ATP concentrations under the same experimental conditions as FIG. 22A.

FIG. 22D depicts a table that comprises Table 1 shows a comparison of method performance for quantitation of ATP spiked in the milk samples using the voltage method

FIG. 23 depicts the Anti-inflammatory and the Pro-inflammatory counter map related to the ATP concentration at zero bias, and the quantum conductance for the “Healthy” HSP60 Sensor 2.

FIG. 24 shows the semi-log plot curve of the ATP concentration ranges effect on the cell Reversed Membrane Potential (RMP) (after subtracting the control). It was shown the trace is not depending upon the ATP concentrations between the range from 100 aM to 800 nM with n=24, eight concentration levels.

FIG. 25 depicts the semi-log plot curve of ATP concentration ranges effect on the ratio of action potential vs. resting membrane potential.

FIG. 26 Left Panel depicts the organic milk CV curves for current vs. applied potential in the presence of 60 nM ATP compared with the milk control (in black color) in 6 consecutive scans at 60 Hz. FIG. 26 Right Panel depicts the CV curves of 60 nM ATP (in red color) in buffer media and curves of the buffer control.

FIG. 27 depicts the DET peaks current vs. scan cycles: the top Panel is for the DET_{red} peak and the bottom Panel is for the DETₒₓ peak.

FIG. 28A depicts the first scan cycle at 60 Hz in the presence of 60 nM ATP (final concentration) in the human milk sample compared with the milk control sample. FIG. 28B depicts the second scan cycle at 60 Hz in the presence of 60 nM ATP (final concentration) in the human milk sample compared with the control milk sample.

FIG. 28C depicts the third scan cycle. FIG. 28D depicts the fourth scan cycle.

FIG. 29 depicts the alternative trends of the superconducting current at the zero-bias vs. scan cycles for the forward scan and backward scan compared with the human milk controls, respectively.

FIG. 30 depicts the overlapping curves of the control human milk sample in 6 consecutive scans vs. the milk sample with 60 nM ATP at 60 Hz.

### Followings are the Brief Descriptions of the Drawings for CIP Application

FIG. 31A depicts the 2D AFM image of the GC/double-layer membrane structure in 1.2×1.2 μm². FIG. 31B depicts the 2D AFM image in small area of 507.1×507.1 nm² with superlattice structures and zinc clusters.

FIG. 31C depicts the 3D view of the AFM image of the zinc cluster “tower” structure. FIG. 31D depicts the 2D view AFM image of the 16.8×16.8 μm² nano porous structured surface. FIG. 31E depicts the roughness analysis results of the large image in FIG. 31D. FIG. 31F depicts the section analysis results of the larger “Five Petal Starflower” nanopores structure in FIG. 31D. FIG. 31G depicts the section analysis results of the small nanopores in a small “five petal star flower” structure in FIG. 31D.

FIG. 32A depicts the 8.4×4 μm2 AFM image of the detail toroidal structure of a large “Five-petal star flower” with zinc ion atoms as “Morning dews” and a fly “Honey bee”. The zinc ion clusters played the role as an “insulator in middle of the Josephson junctions. FIG. 32B depicts the 2D view of the layered membrane and the “fly bee” of zinc clusters in 3.2×3.2 um². FIG. 32C depicts peak depth analysis of the larger “Five petal star flower” pore depth.

FIG. 33 depicts the Friedel-oscillation AFM image in the 1.3×1.3 μm2 view window.

FIG. 34A depicts the toroidal Flexible Josephson junction array circuitry model. FIG. 34B depicts the art of the layered “Five petal star flower” array matrix layout on a GC electrode.

FIG. 35 depicts the art model in a side view of the Josephson Junction. “60” is the GC electrode with a nanopore and pillar membrane; “61” is a symbol indicates the two current collector's connection is reversable. “62” refers to another layer of coating on the membrane embedded with o-nitrophenyl acetate (o-NPA) on top of the GC/MEA; “63” refers to the Pt current collectors; “64” refers to the collagen-1 coating matrix on a dielectric insulator; “65” refers to the circular current flow in a positive direction with the zinc atoms as the brown balls; “66” refers to the cyclodextrin linked copolymer formed “Five petal Starflower” matrix alignment with each other produced the eternal superconducting current in the blue circle having induced a Φ₀, single flux quantum, that a non-ferromagnetic field is produced; “67” refers to the Cooper pair. Notice there is an air barrier between the membrane and the array of cyclodextrin matrix. “68” refers to the PEG. . . . PVP's N-terminal chain. The righthand side is an illustration of the JJ circuit.

FIG. 36A depicts the collagen concentrations impact on the i-V curves at zero-bias at 10 KHz scan rate compared with the control with the GC/S-I-M whole cell configuration. FIG. 36B depicts the collagen concentrations impact on the i-V curves at zero-bias at 20 KHz scan rate compared with the control with the GC/S-I-M whole cell configuration. The label I_{c}⁺ indicates the positive critical supercurrent at zero bias; while the I_{c}⁺ indicates the negative critical supercurrent at zero bias for the control sample.

FIG. 37A depicts the plot of the initial rate of supercurrent in nA/(μg/mL) due to collagen at 10 and 20 kHz for the backward scan over collagen concentration 0.5 ng/mL to 50 μg/mL compared with controls. respectively. FIG. 37B depicts the plot of the initial rate of supercurrent in nA/(μg/mL) due to coated collagen at 10 and 20 kHz for the forward scan over collagen concentration 0.5 ng/mL to 50 μg/mL compared with the controls. respectively. FIG. 37C depicts the plots of supercurrent vs. collagen concentrations at 10 kHz scan rate for a comparison between a whole cell and a half-cell's performance against the controls. FIG. 37D depicts the plots of supercurrent vs. backward scan rates between 60 Hz and 20 kHz for a half-cell over collagen concentrations from 0.5 ng/mL to 50 μg/mL (6 levels) compared with two controls.

FIG. 38A depicts the collagen concentrations impact on the i-V curves of a Josephson diode at zero-bias at 1 kHz scan rate blocking the current flow direction compared with the control, and the label I_{c}⁺ indicates the positive critical supercurrent at zero bias; while the I_{c}⁺ indicates the negative critical supercurrent at zero bias for the control sample. FIG. 38B depicts the collagen concentrations impact on the i-V curves at zero-bias of a Josephson diode at 300 Hz scan rate compared with the control. FIG. 38C depicts the collagen concentrations impact on the i-V curves of a whole cell at zero-bias at 60 Hz scan rate compared with the control.

FIG. 39 depicts the i-V curves of a half-cell of GC/S-I-N at 60 Hz scan rate compared with control, and no media. “Blue” arrows indicate the forward scan, and the black arrows for backward scan.

FIG. 40A depicts the i-V curves of the half-cell in FIG. 39 has 10 consecutive cycles scan at 60 Hz for with or without collagen. FIG. 40B depicts the i-V curves of a whole cell of GC/S-I-M/GC shown in FIG. 38C has 10 consecutive cycles scan at 60 Hz over collagen coating from 0.5 ng/ml to 25 μg/mL compared with control.

FIG. 41A depicts the plot of supercurrent at zero-bias vs. time for 10 cycles scans with or without collagen coating from the half-cell configuration at 60 Hz scan rate at the backward scan. FIG. 41B depicts the same types of plots of supercurrent at zero-bias vs. time for 10 cycles scans with or without collagen coating from the whole cell configuration at the backward scan. FIG. 41C depicts the i-V curves of a GC/SIS sensor as control over the scan rate 60 Hz to 20 KHz with no media, no dielectric insulator, and no reagent conditions. This sensor doesn't contain a dielectric insulator. The red arrows indicate a forward scan, and the black arrows backward scan. Arrows at zero bias indicates the state.

FIG. 42A depicts the i-V curves of the GC/SIS sensor control in 10 KHz consecutive scan cycles in no media, no dielectric insulator, and no reagent conditions. The “SIS” means the “I” is the zinc ion clusters acted as “insulator”. The polymer superlattice as a superconductive material with a symbol is “S”. FIG. 42B depicts the i-V curves of the GC/SIN/Pt half-cell control with a coated insulator in 10 KHz consecutive scan cycles under the same experimental conditions as FIG. 42A. The “N” of “SIN” means the “normal metal” of Pt. “I” means the dielectric insulator, and “S” means the polymer superlattice membrane is superconductive. FIG. 42C depicts the i-V curves of the GC/SIN/Pt half-cell at 10 KHz scan rate with collagen coating over 0.5 ng/ml to 50 μg/mL compared with the control with 10 consecutive cycle scans. FIG. 42D depicts the i-V curves of the GC/SIM/GC whole cell at 10 KHz scan rate with collagen coating over 0.5 ng/mL to 50 μg/mL compared with the control with 10 consecutive cycles scans. FIG. 42E depicts the plot of supercurrent at zero-bias vs. time among 10 cycles scans with or without collagen coating from the half-cell configuration at 10 KHz scan rate at the forward scan. And also against the control sensor's supercurrent at zero bias. FIG. 42F depicts the same types of plots of supercurrent at zero-bias vs. time for 10 cycles scans with or without collagen coating with the whole cell GC/SIM/GC configuration and against the control sensor at the forward scan at 10 KHz.

FIG. 43 depicts the plots of quantum conductance G_{c}/Gₒ vs. applied potential from the sample with 150ng/ml collagen coating on the insulator compared with the control at the first and the 10^{th} scan cycles at 10 KHz scan rate.

FIG. 44A depicts the plots of peak current at zero voltage vs. phase (Φ₀/π) in a total 0.10 s in the presence of collagen coating over concentration 0.5 ng/ml to 50 μg/mL on insulators of a half-cell compared with the controls. FIG. 44B depicts the plots of peak current at zero voltage vs. phase (Φ₀/π) in a total 0.10 s in the presence of collagen coating over concentration 2.4 μg/mL to 50 μg/mL on insulators of a whole cell compared with the control.

FIG. 45A depicts the 3D dynamic map of quantum conductance vs. potential and vs. supercurrent for the control Josephson junction array device, which has no coating on the insulator at the first scan cycle at 10 KHz scan rate. FIG. 45B depicts the contour map of quantum conductance vs. potential, and vs. supercurrent for the control Josephson junction array device, which has no coating on the insulator, at the first scan cycle at 10 KHz scan rate. FIG. 45C depicts the 3D dynamic map of quantum conductance vs. potential and vs. supercurrent for the control Josephson junction array device, which has no coating on the insulator at the 10^{th} scan cycle at 10 KHz scan rate. FIG. 45D depicts the contour map of quantum conductance vs. potential, and vs. supercurrent for the control Josephson junction array device, which has no coating on the insulator, at the 10^{th} scan cycle at 10 KHz scan rate.

FIG. 46A depicts the 3D dynamic map of quantum conductance vs. potential and vs. supercurrent with 150 ng/mL collagen coated insulator at the GC/S-I_{Collagen}-M/GC Josephson junction array device at the first scan cycle at 10 KHz scan rate. FIG. 46B depicts the contour map of quantum conductance vs. potential and supercurrent for 150 ng/ml collagen coated insulator at the GC/S-I_{Collagen}-M/GC Josephson junction array device at the first scan cycle at 10 KHz scan rate. FIG. 46C depicts the 3D dynamic map of quantum conductance vs. potential and vs. supercurrent with 150 ng/mL collagen coated insulator at the GC/S-I_{Collagen}-M/GC Josephson junction array device at the 10^{th} scan cycle at 10 KHz scan rate. FIG. 46D depicts the contour map of quantum conductance vs. potential and supercurrent with 150 ng/ml collagen coated insulator in the GC/S-I_{Collagen}-M/GC Josephson junction array device at the 10^{th} scan cycle at 10 KHz scan rate.

FIG. 47 depicts the device finished 9000 cycles of charge/discharge at ±30 mA with 50 ms per cycle under media-free and electrolyte-free conditions at room-temperature.

FIG. 48 depicts the plot curve of cell voltage vs. time of the GC/SIM/GC device with 150 ng/ml collagen coated on each side of the insulator continued discharge at 50 mA at a nominal voltage 5.4 V for 16.6 hours after finished 9000 charge/discharge cycles at media-free and electrolyte-free conditions.

FIG. 49A depicts the stability comparison of a single cycle (50 ms) for charge/discharge at the first cycle with the collagen 150 ng/ml coated GC/SIM/GC device compared with the control, that without collagen. FIG. 49B depicts the voltage vs. time curve after 9000 cycles charge/discharge. FIG. 49C depicts the voltage vs. time curve after the 9000 cycles in FIG. 49B and after rest 21 days' testing.

FIG. 50 depicts the contour plot of the collagen concentration impact on Eⱼ and E_{L}.

FIG. 51 depicts the contour map plot of the capacitive energy of the device as a memcapacitor vs. the nonlinear potential energy stored in the LJJ array, and vs. collagen concentrations.

### EXAMPLE 1

### Fabrication of the Membranes

Sensor 1 has an activated biomimetic MMP-2 membrane by a heating method at 80° C. for 5 minutes using the innate biomimetic MMP-2 membrane fabricated based on a published procedure [34]. Sensor 2 was also in a state of activation of biomimetic MMP-2 by a direct deposited method with compositions of TCD, PEG, PVP, bM-β-DMCD, and embedded zinc chloride on gold chips with appropriate proportions at 370C for 96 hours. The morphology of the AU/SAM was characterized using an Atomic Force Microscope (AFM) (model Dimension Edge AFM. Bruker. MA).

### EXAMPLE 2

### The Friedel-Oscillation in the Superlattice Membranes

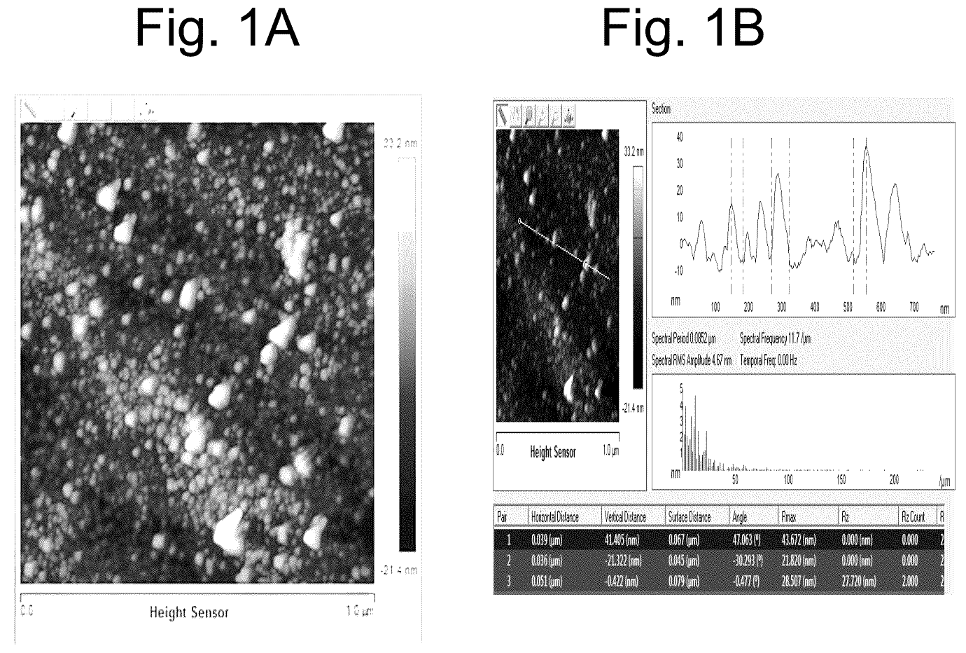

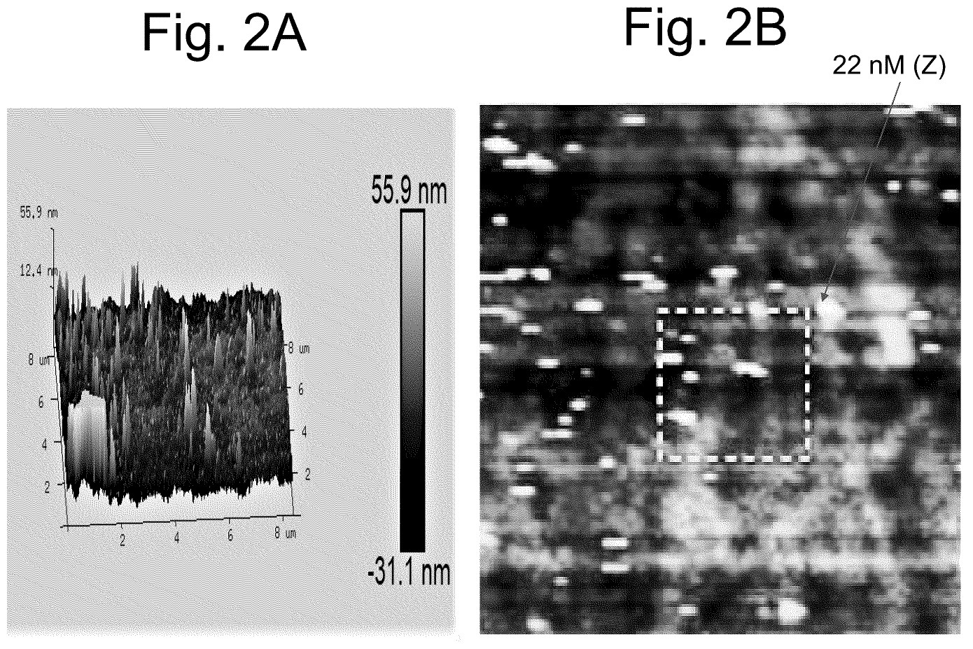

Friedel oscillation is a phenomenon of long-range indirect interactions between electrons on a superlattice surface [21]. Evaluations of the Friedel-oscillation were conducted based on the AFM images. FIG. 1A revealed the strong Friedel-oscillation with a flame-like electric cloud surrounding the zinc atoms which the “cloud” moves toward the same direction in the 1.0 μm² area. This is the evidence of the Cooper pair transmission waves at the Josephson junction. The image was for the biomimetic “native” MMP-2 membrane, i.e., the membrane comprised of triacetyl-β-cyclodextrin (TCD), polyethylene glycol diglycidyl ether (PEG), poly(4-vinyl pyridine) (PVP), bis-substituted imidazole dimethyl-β-cyclodextrin (bM-β-DMCD), cysteine and embedded zinc chloride in appropriate proportions. The native MMP-2 protein has two states: an innate state with the cysteine “on” and an activated state with the cysteine “off”. FIG. 1A depicts the 2D AFM membrane image in the native state of the biomimetic MMP-2 device. FIG. 1B depicts the cross-section analysis of the AFM membrane image. FIG. 1A was with the cysteine “On” in its innate state, and we observed the Cooper pair electrons moving toward the same direction. FIG. 2A depicts the 3D AFM image of the activated biomimetic MMP-2 Sensor 1 by the heating method. FIG. 2B depicts the enlarged 2D AFM image with the same z-value range between 33.2 to-21.4 nm as the native state device in FIG. 1A for comparison. Sensor 1 has an activated biomimetic MMP-2 membrane by a heating method to kick out the cysteine group in the network, as evidence, we did not observe moving Cooper pairs in FIG. 2A, the labeled square has the same Z range as shown in FIG. 1A. The matrix of the superlattice was shown partially damaged shown in FIG. 2B.

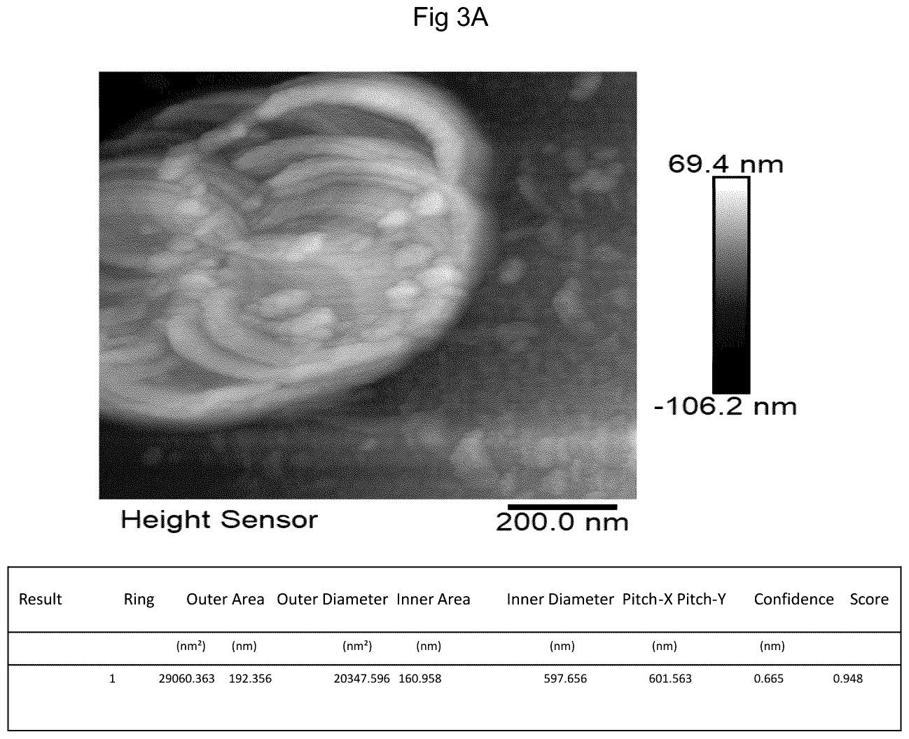

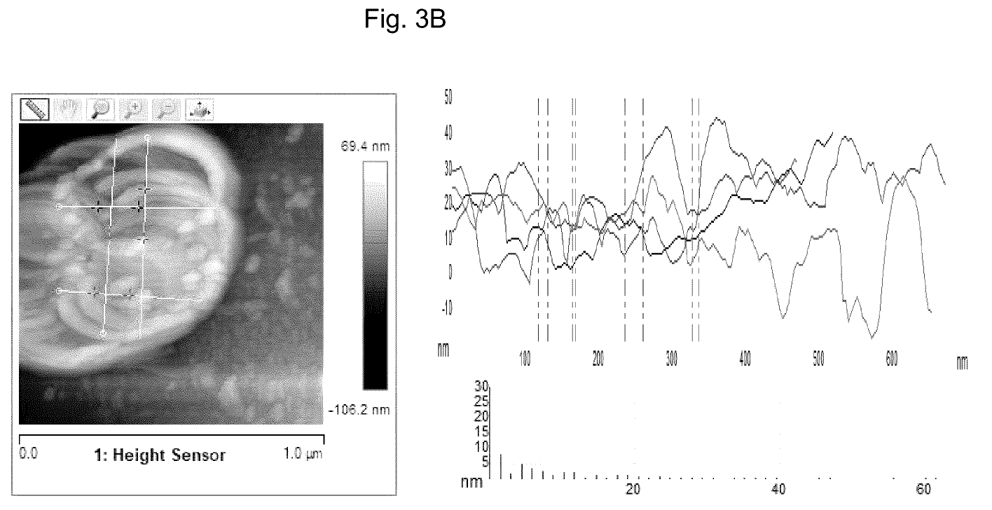

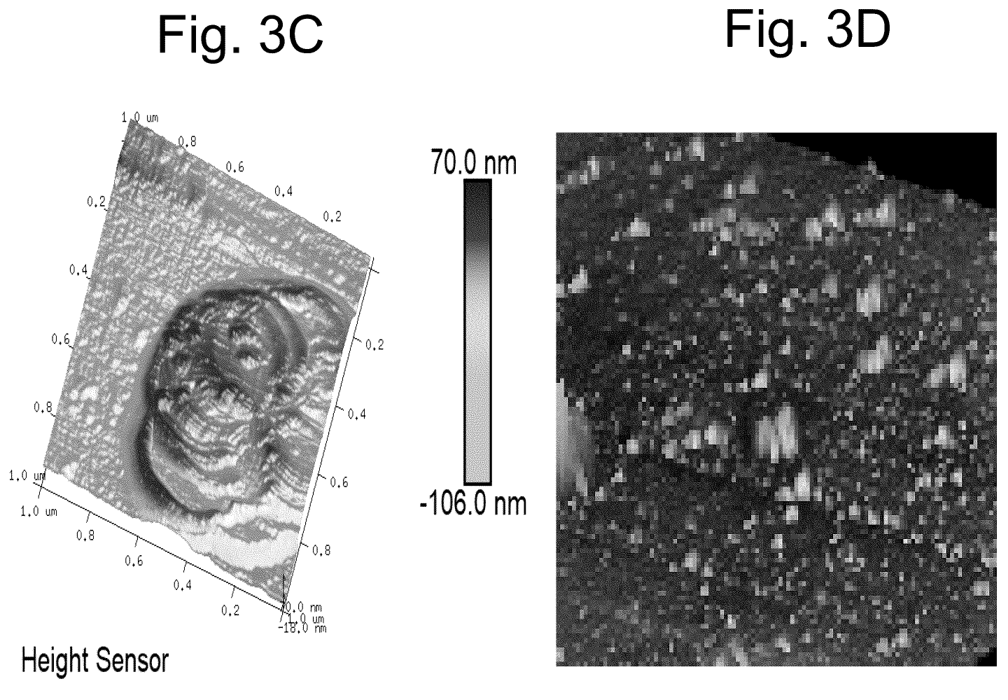

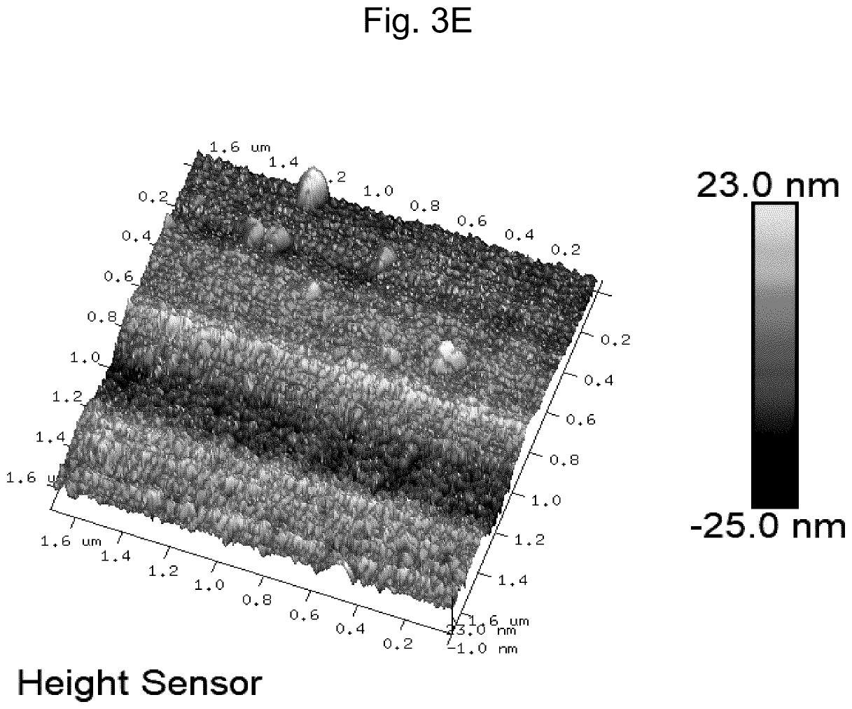

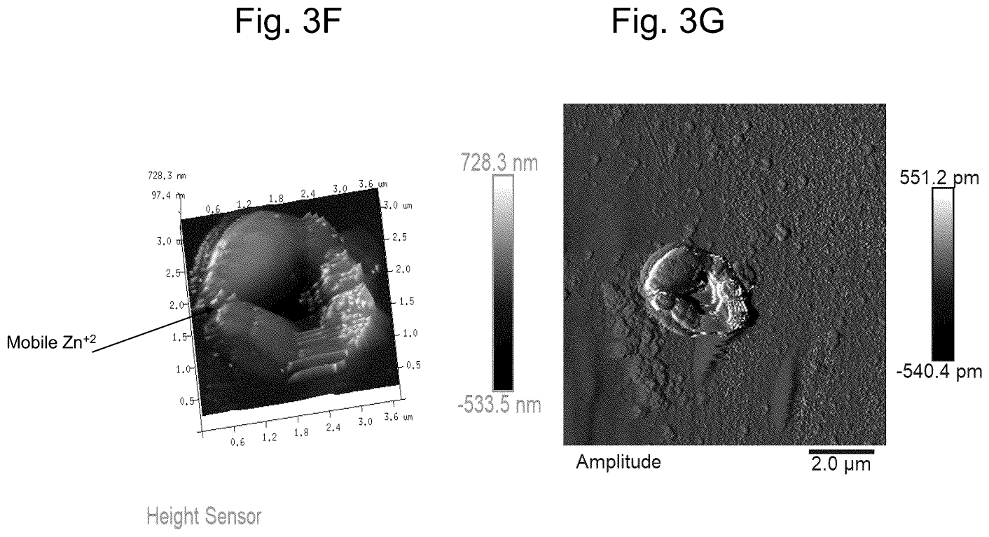

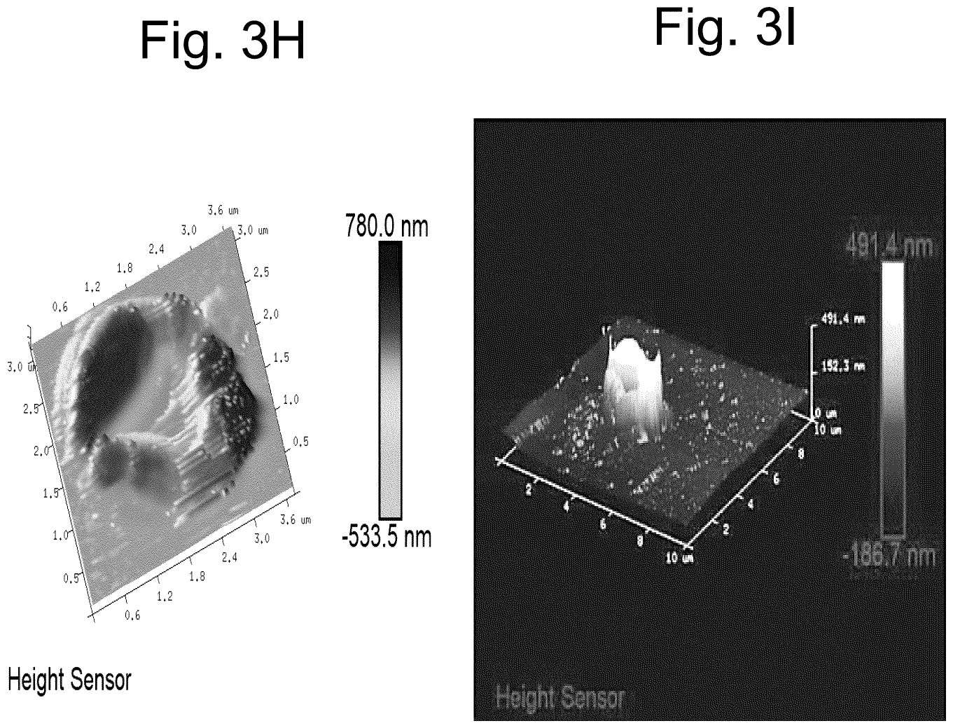

FIG. 3A depicts the 2D image of the multiple-layer ring structure of the activated biomimetic MMP-2Sensor 2 by the direct fabrication method in 0.734×0.734 μm². The ring structure parameters labeled as “1” are listed. The inner ring diameter is about 161 nm, and the out ring diameter is 192 nm. Sensor 2 was directly fabricated using the same polymers and other compositions such as bM-β-DMCD/TCD/PEG/PVP/ZnCl², except without cysteine. The Friedel-oscillation was observed in two locations in FIG. 3A. FIG. 3B depicts the cross-section analysis of the AFM membrane image. FIG. 3C depicts the 3D AFM image from the bird's view. FIG. 3D depicts the enlarged image in 0.9×0.9 μm² shown in the flat area in FIG. 3I. The Friedel-oscillation observed as the moving flames surrounded the zinc atoms, while the Cooper pairs moved toward the same direction and the superlattice matrix was very orderly arranged on the surface of the FIG. 3D. FIG. 3E depicts the alternative arrangement between the valley band and the flat band on the membrane which promotes the Cooper-pair electron cloud mobility in 1.6×1.6 μm². FIG. 3F depicts the enlarged 2D AFM image at high sensor mode in a top view of the biomimetic Heat Shock Protein (HSP) 60 chaperone structure having 7 subunits as shown on the top. The arrow indicates the mobile zinc ions. FIG. 3G depicts the top view in an amplitude mode of the AFM image of the large single porous biomimetic HSP60 structure. FIG. 3H depicts the side view of the 3D AFM image in high sensor mode for the single porous structure in 3×3.6 μm². FIG. 3I depicts the 3D AFM image of the tall cylinder structure in a size of 1.3-1.5 um diameter and 491 nm in height of the biomimetic HSP60 in 10×10 μm².

### EXAMPLE 3

### Direct Electron Relay (DER) Model Based on a Cylinder Cage-like Structured Polymer Network

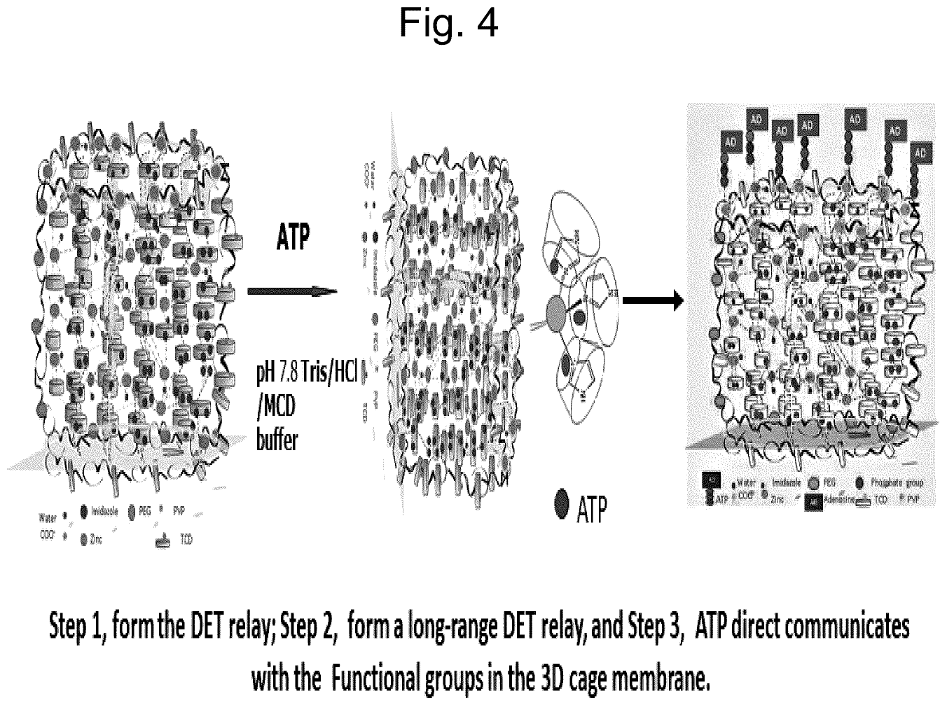

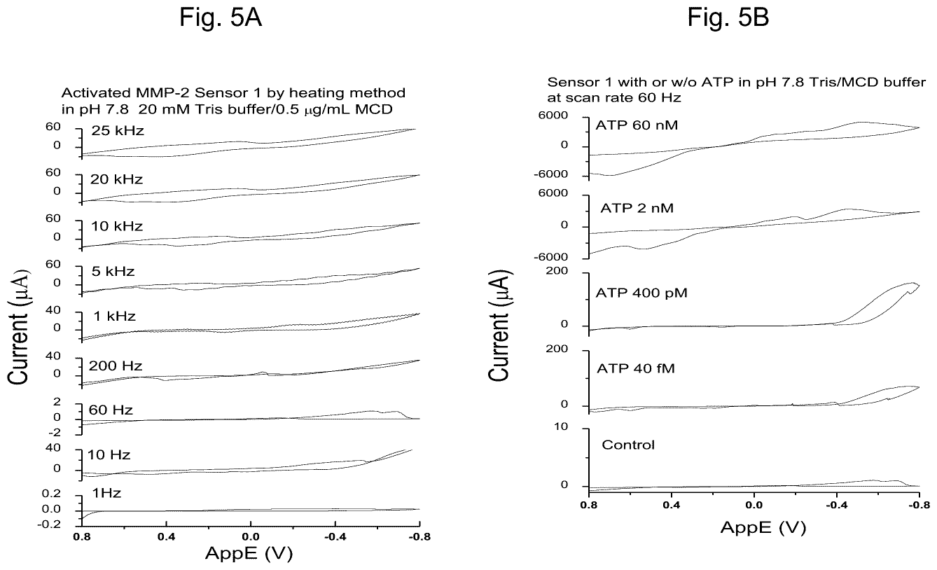

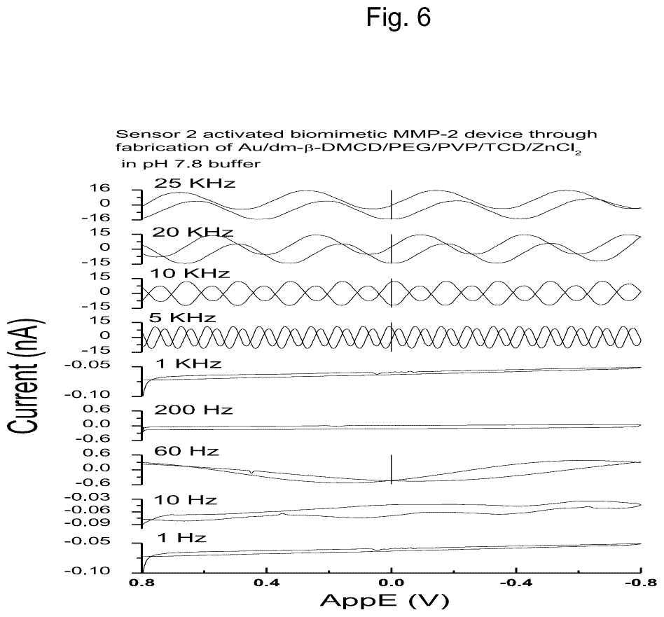

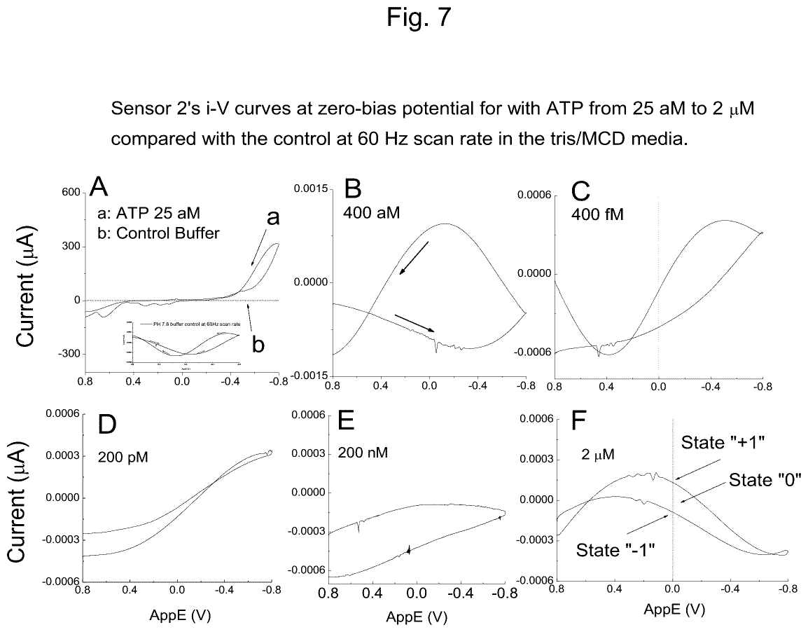

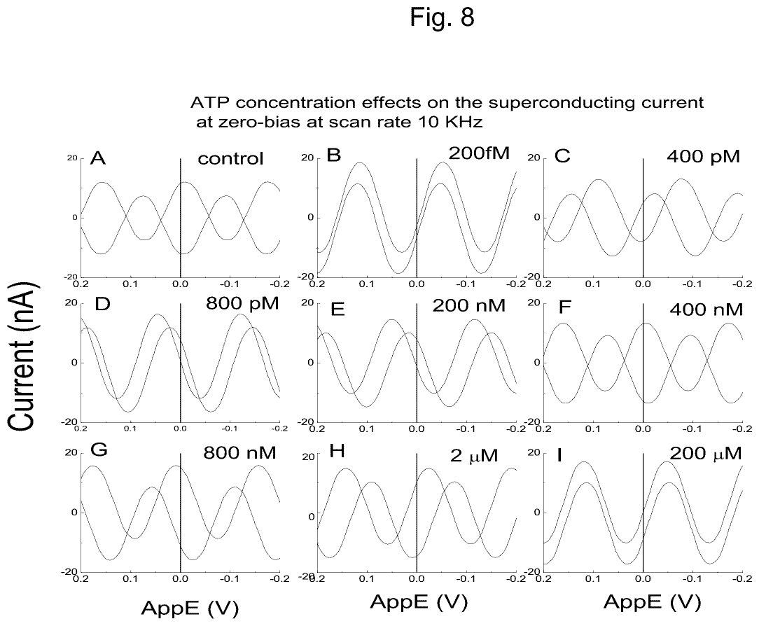

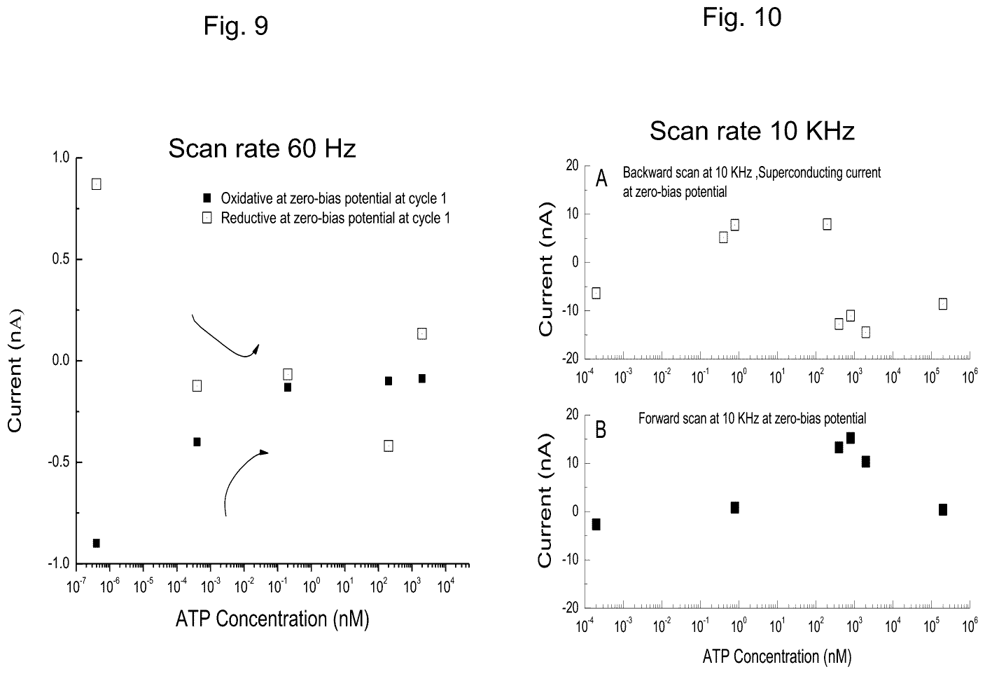

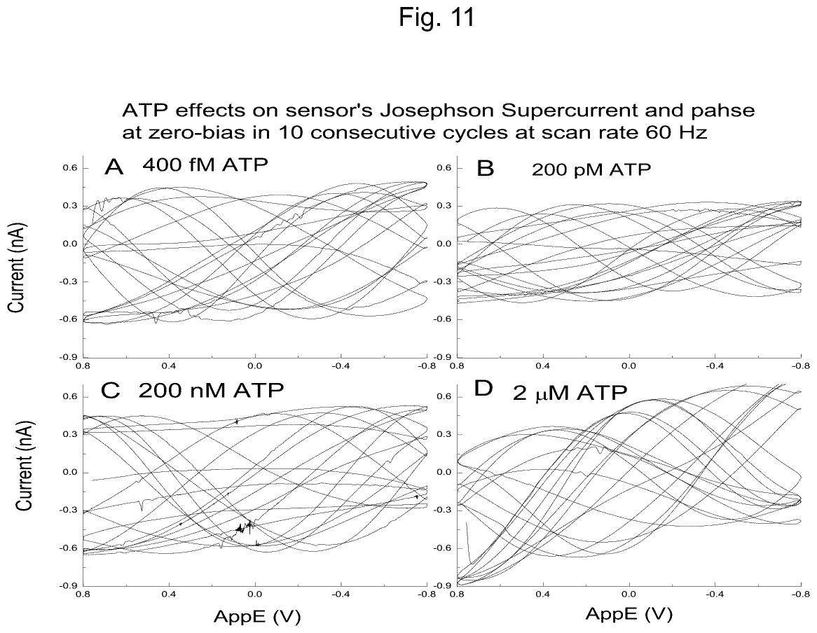

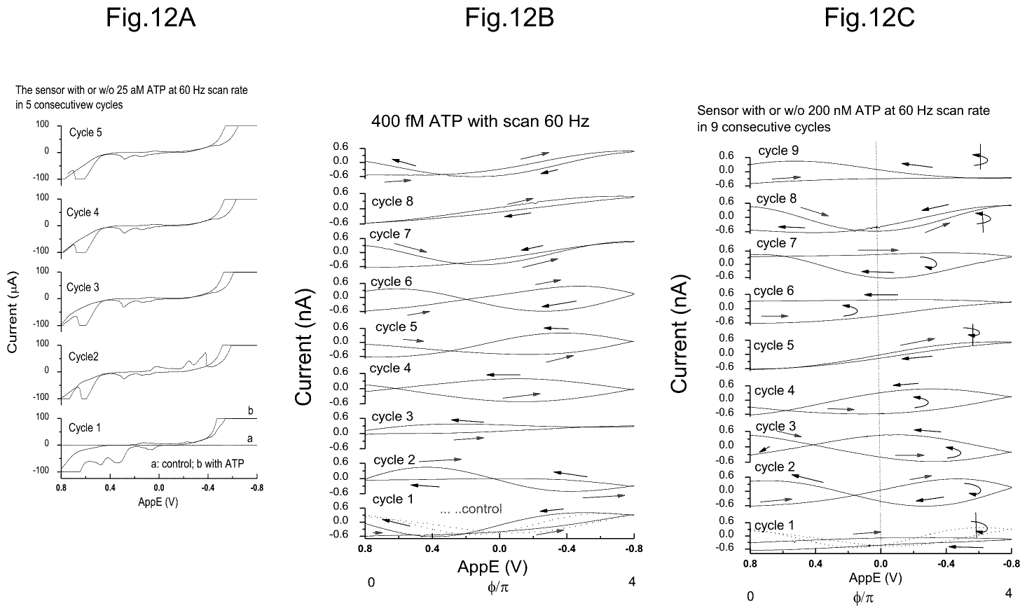

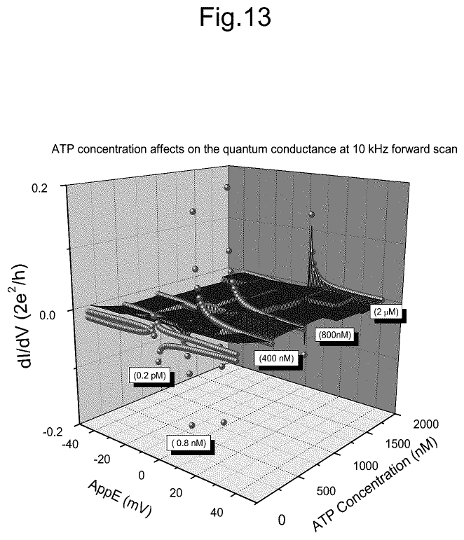

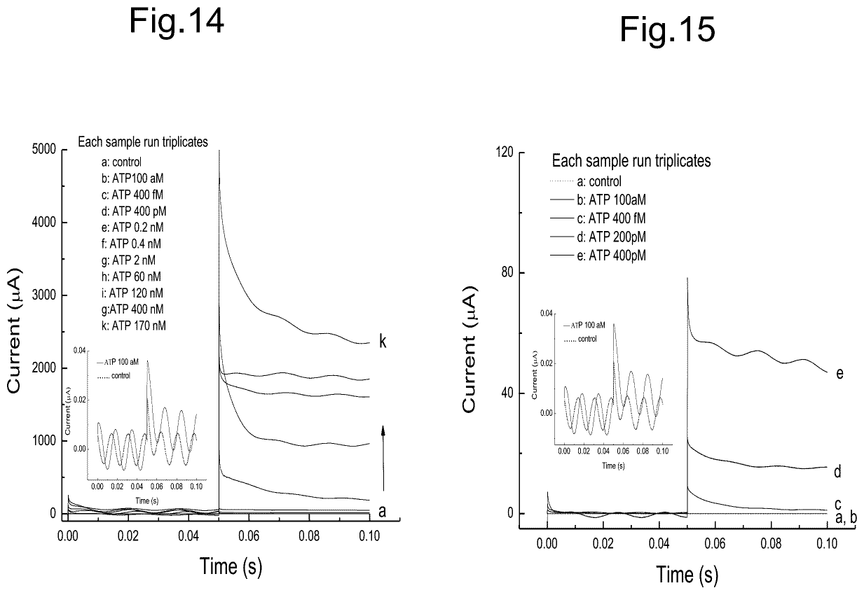

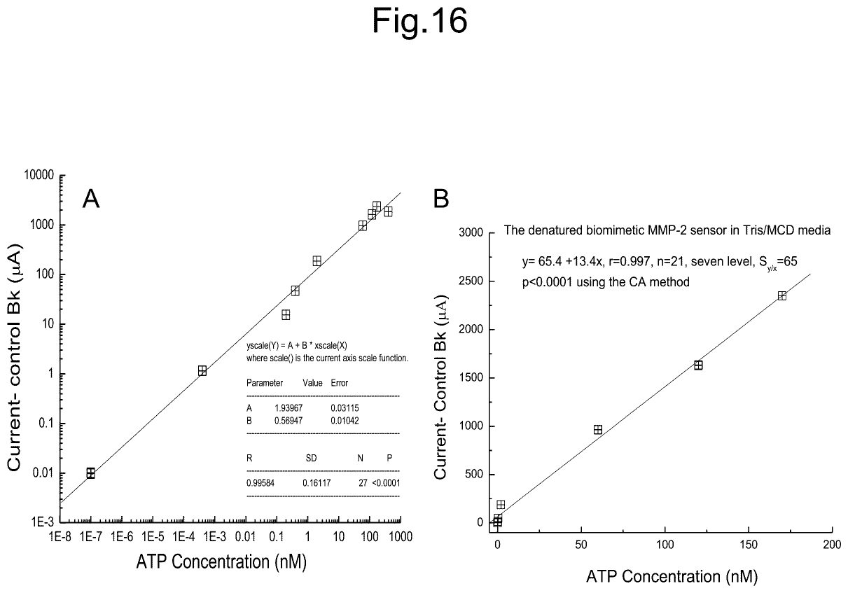

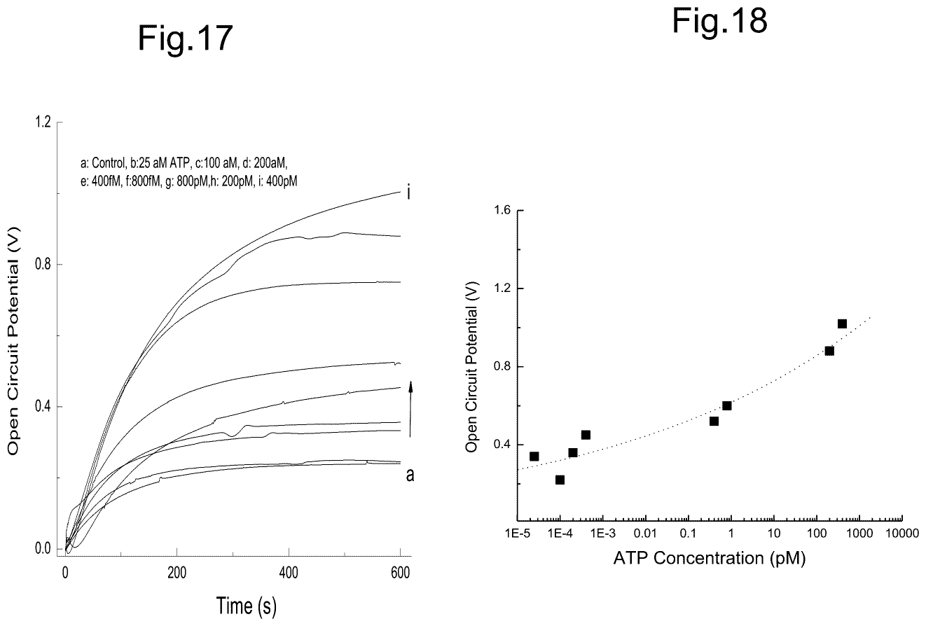

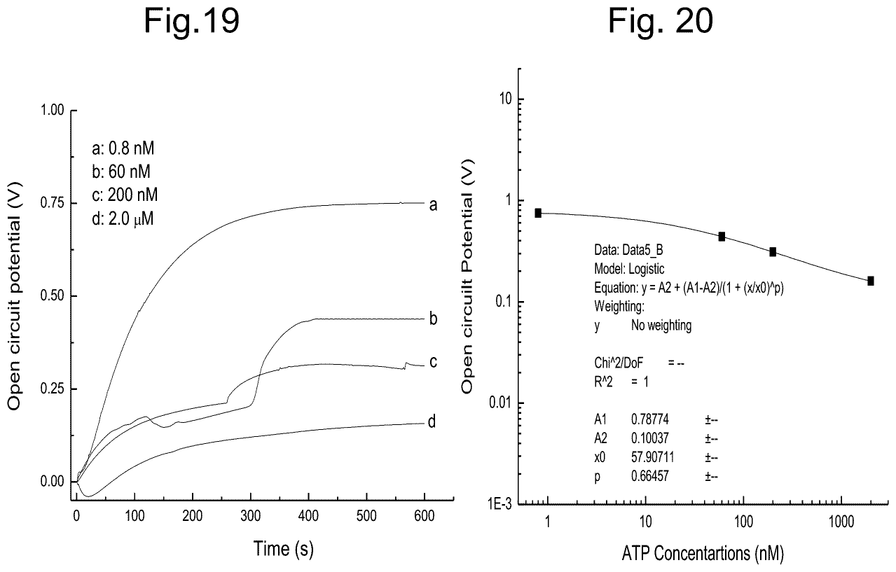

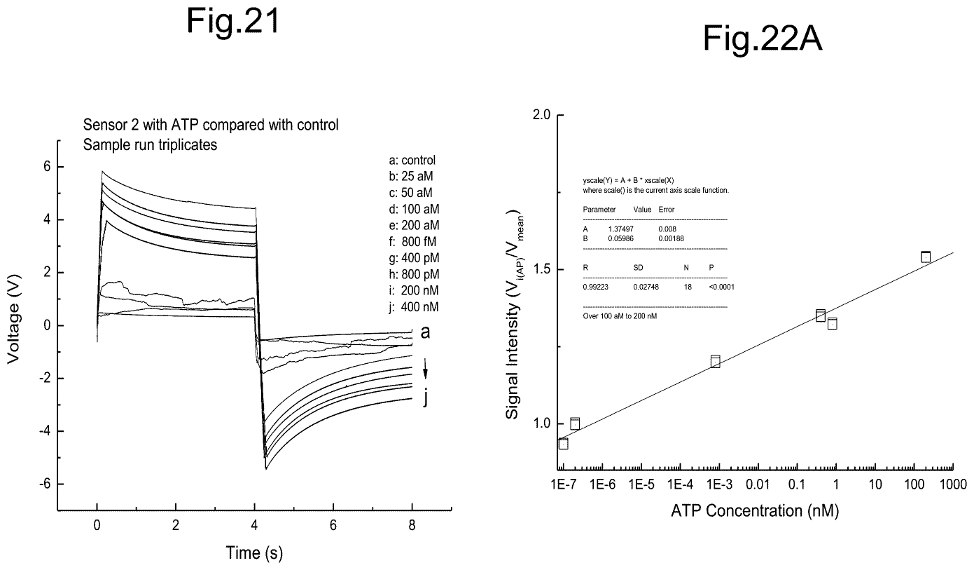

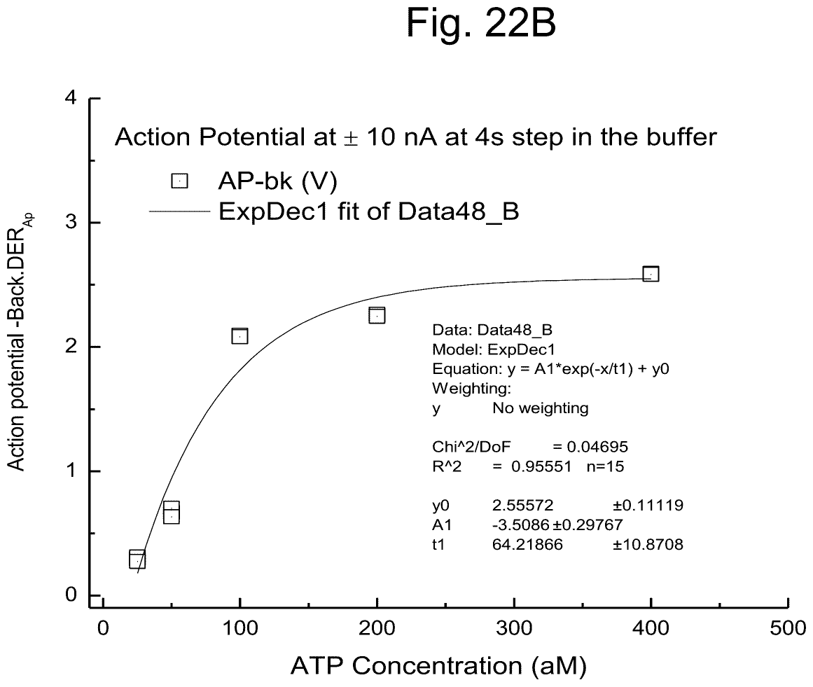

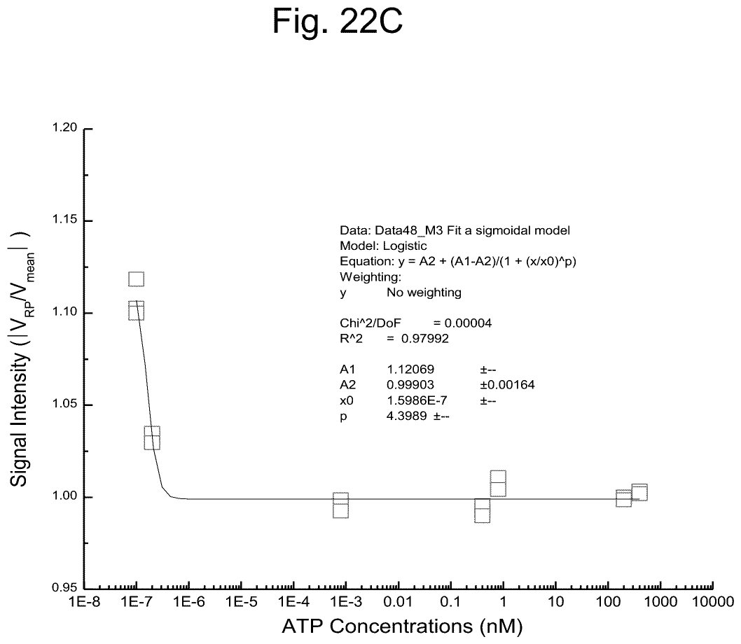

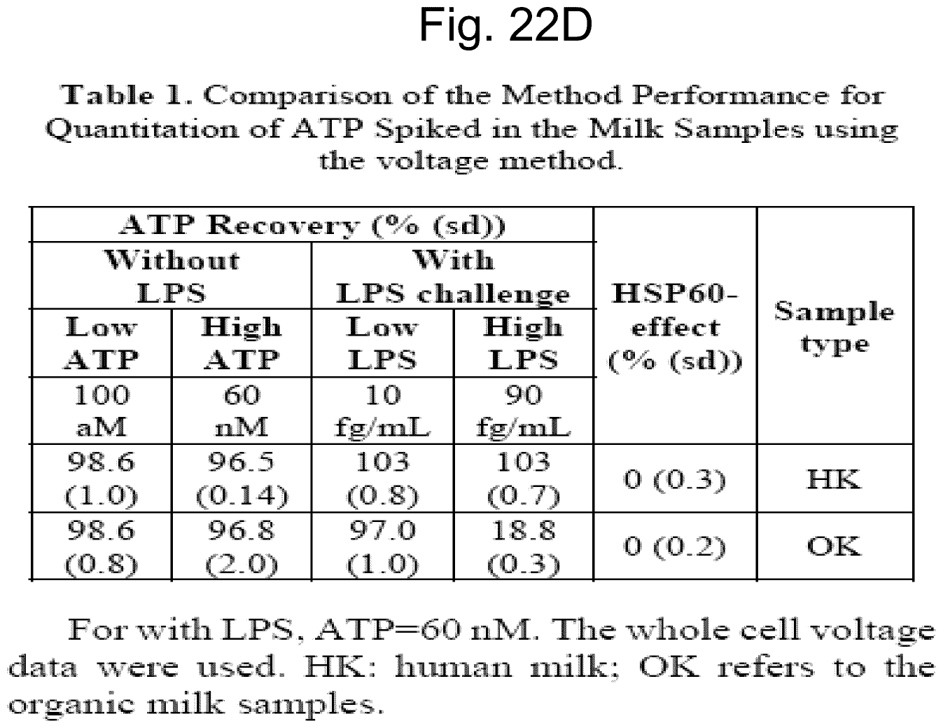

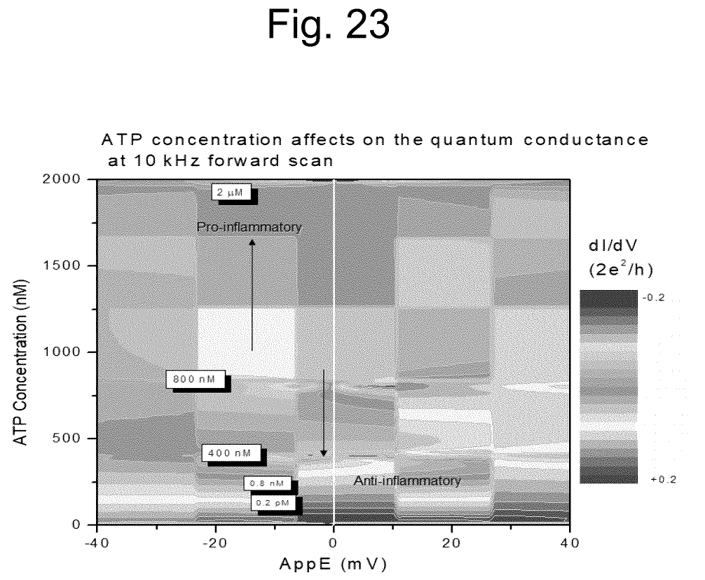

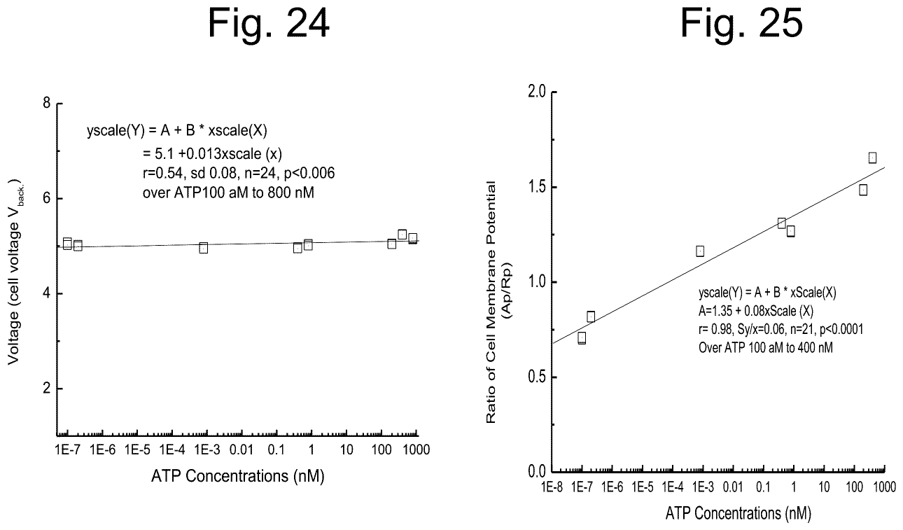

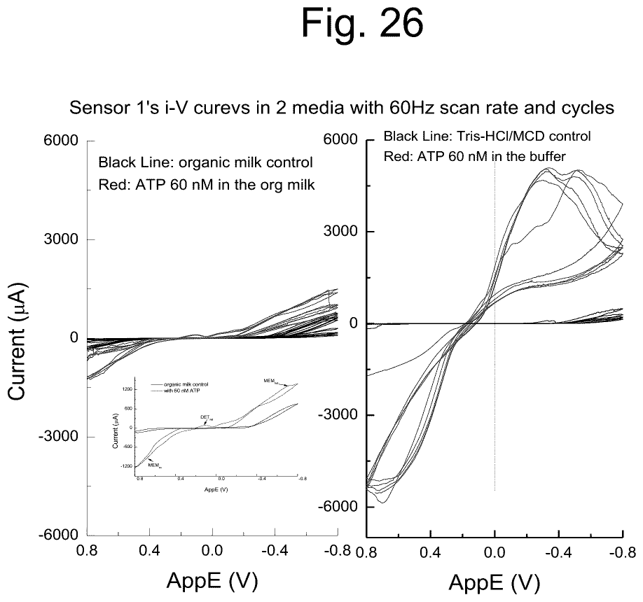

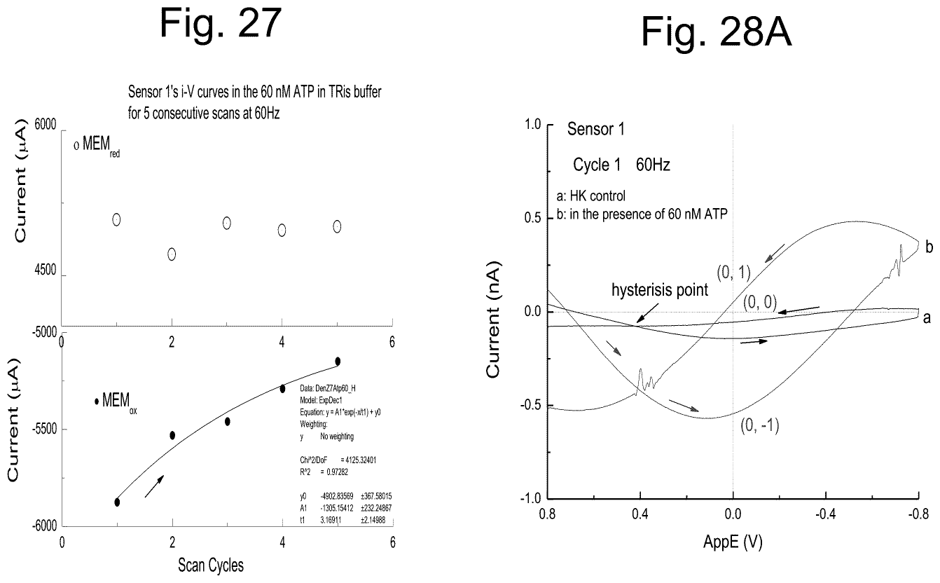

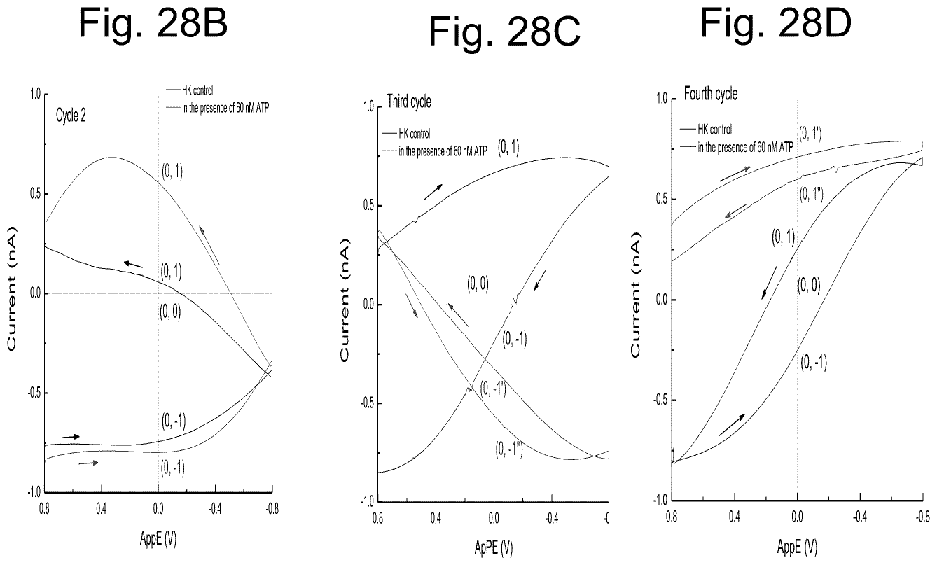

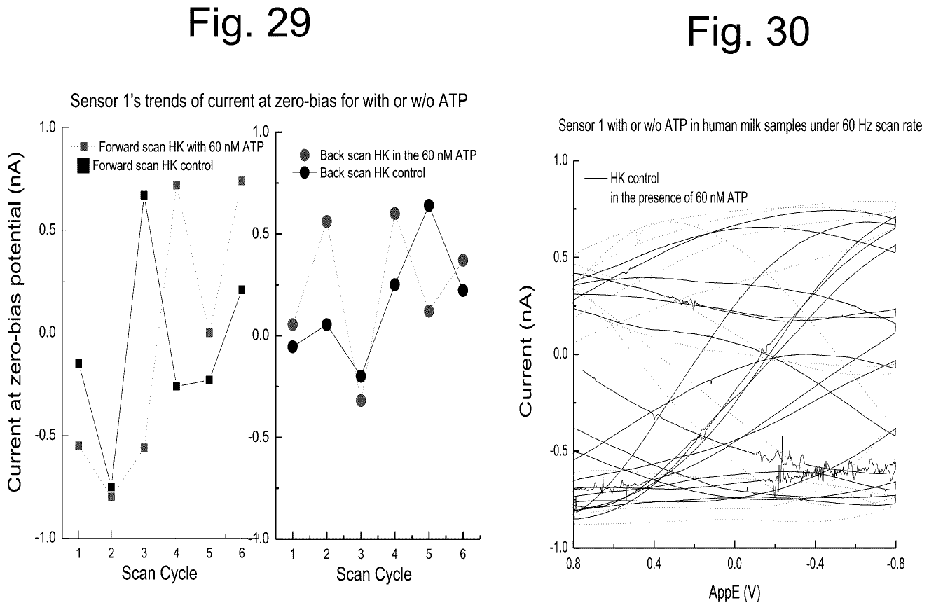

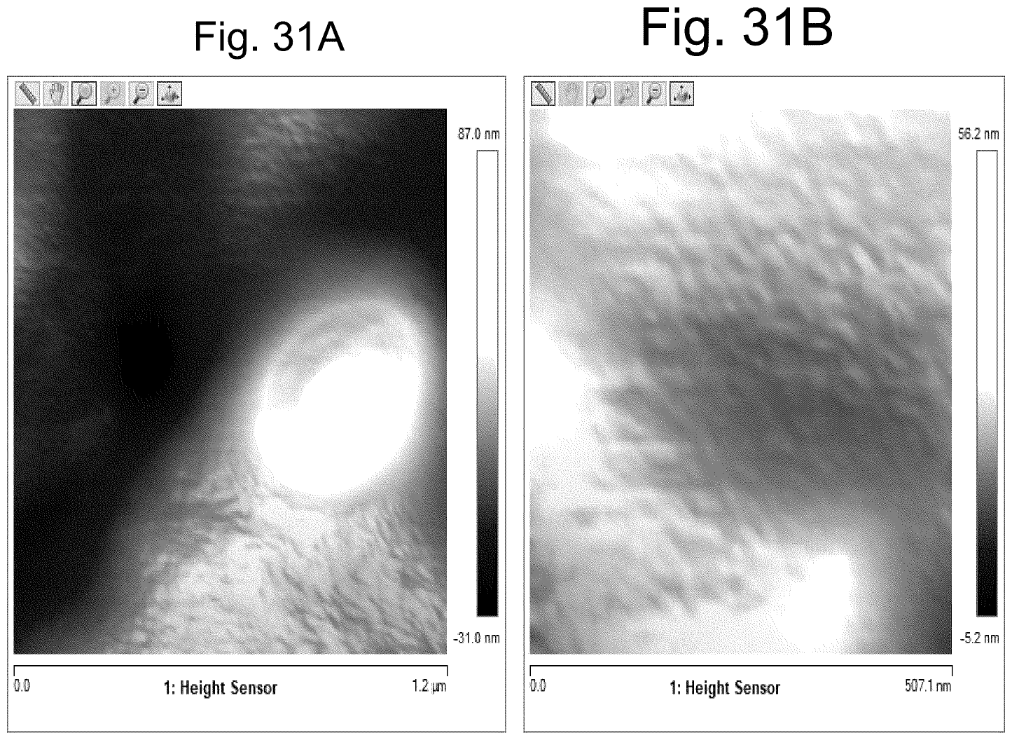

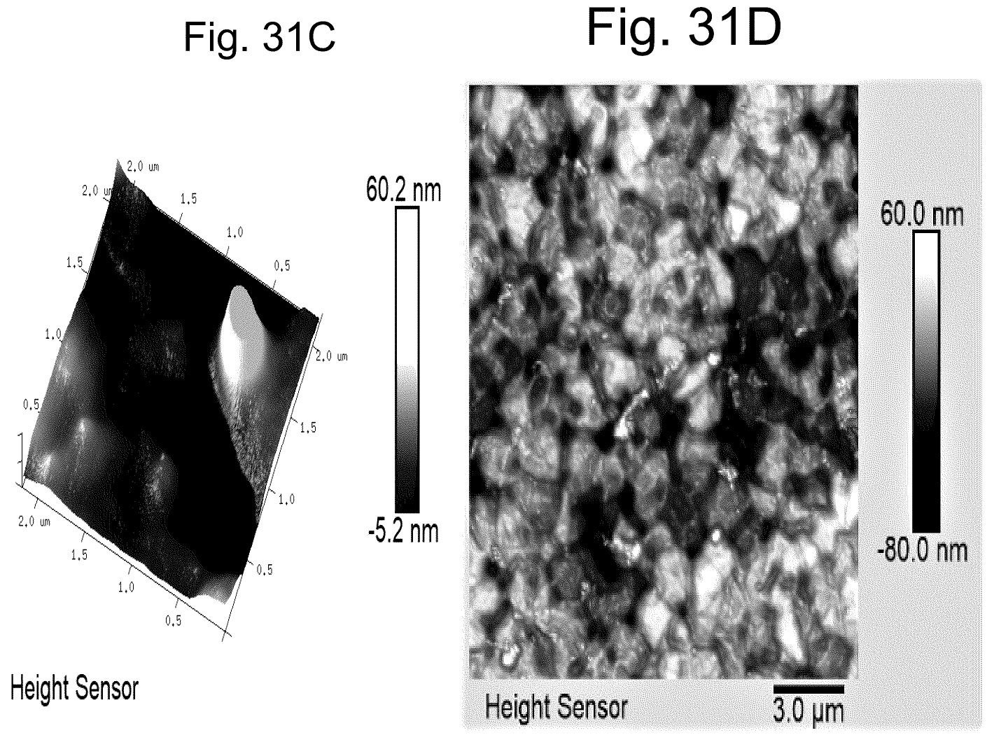

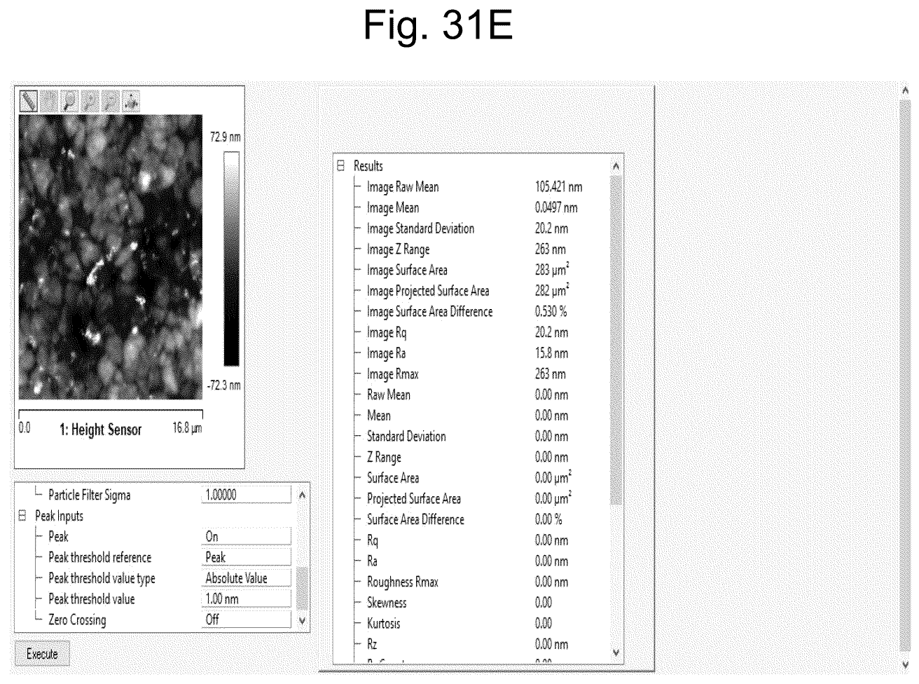

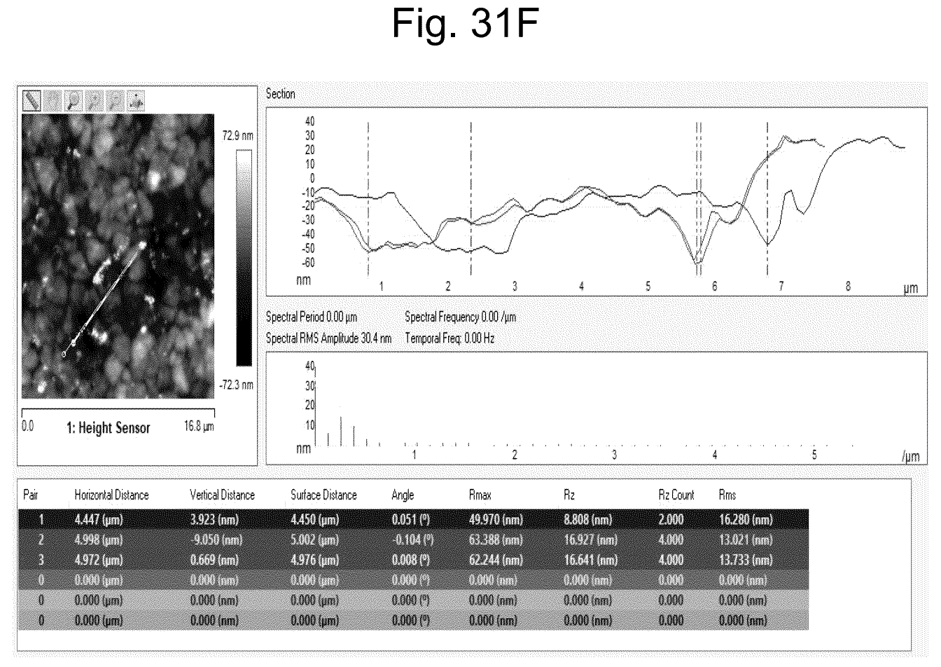

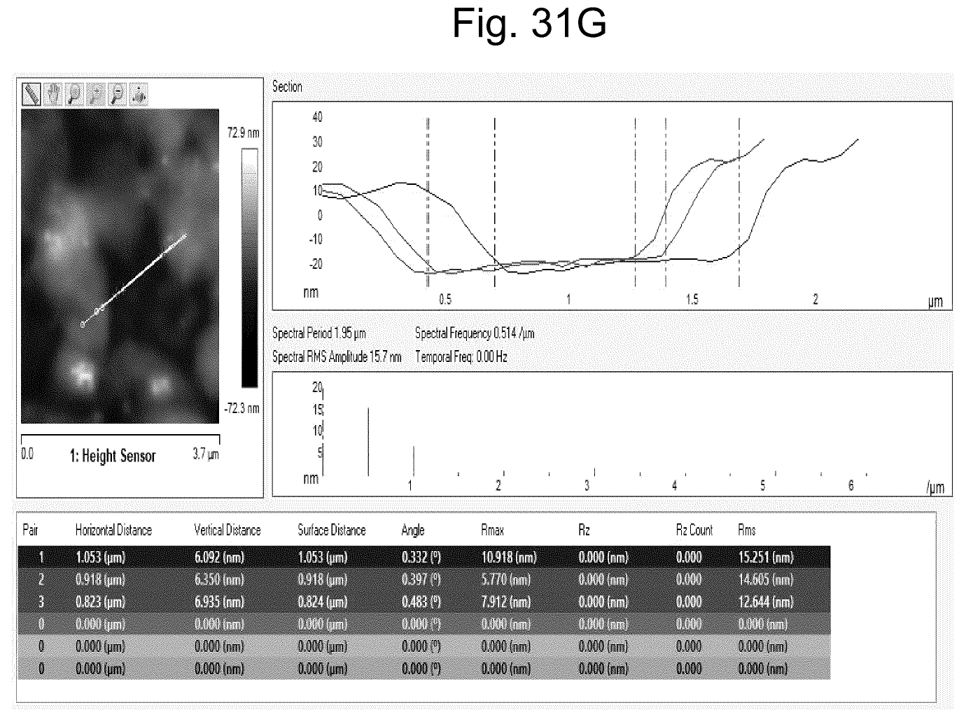

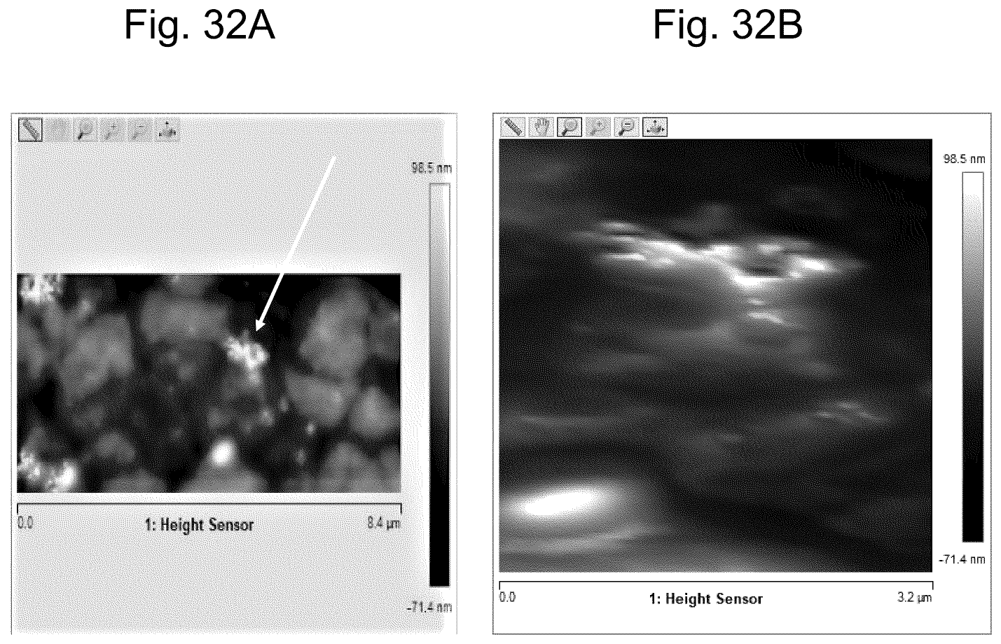

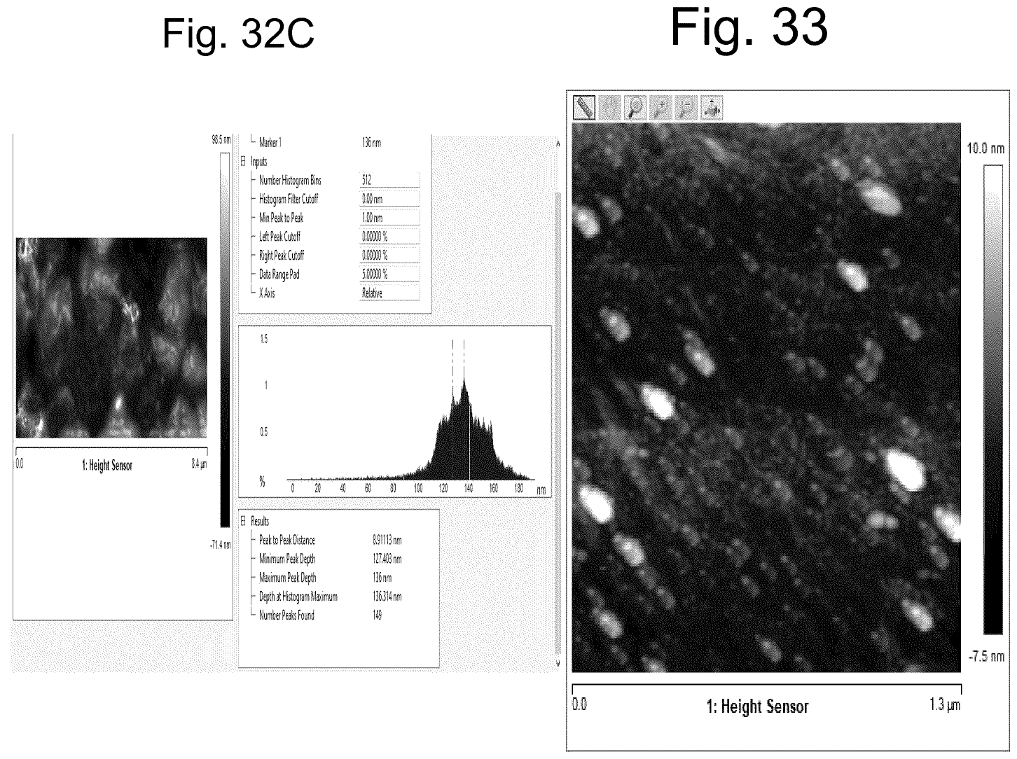

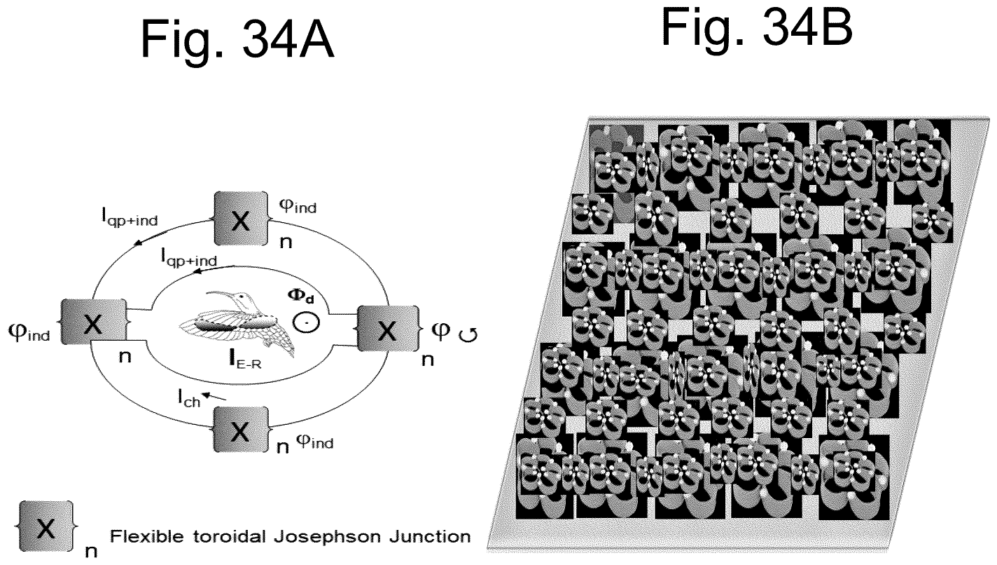

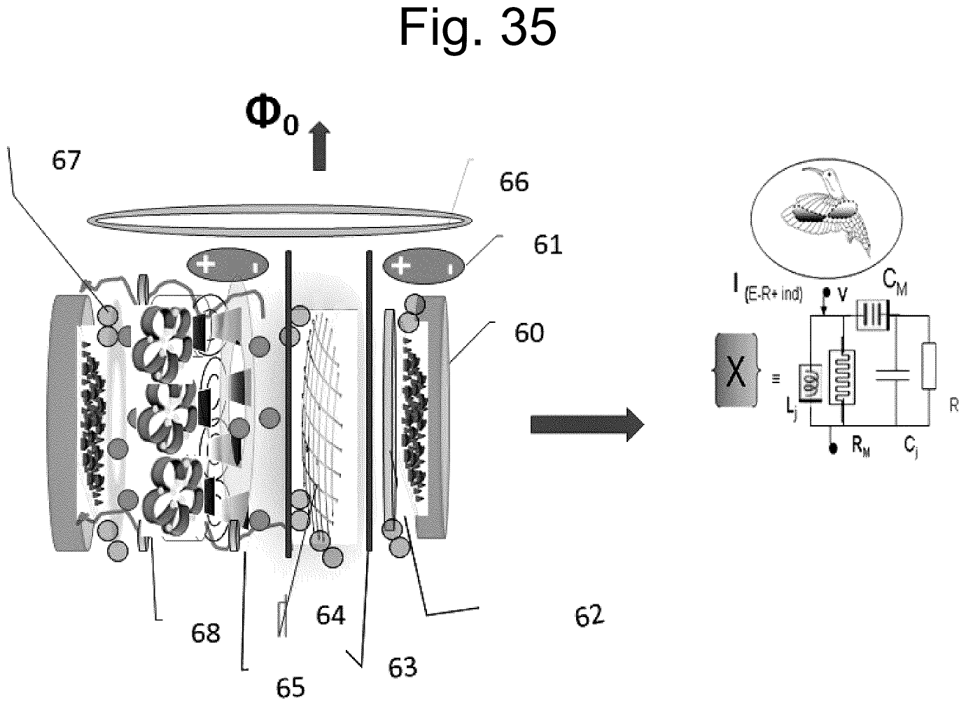

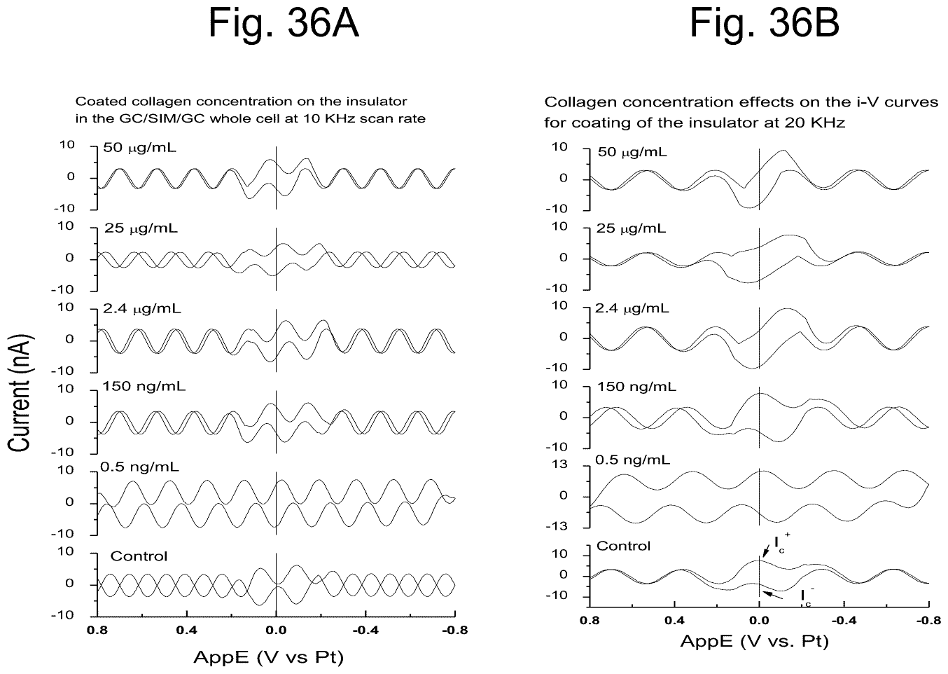

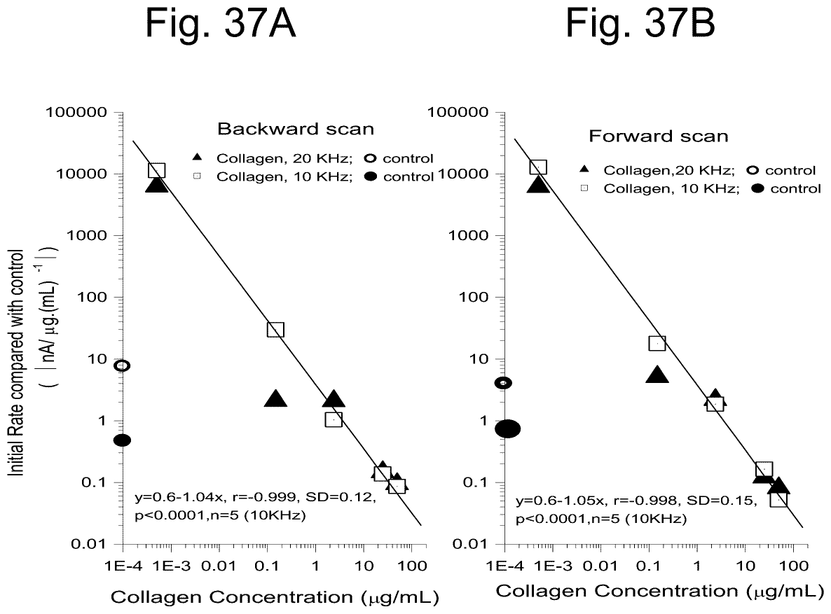

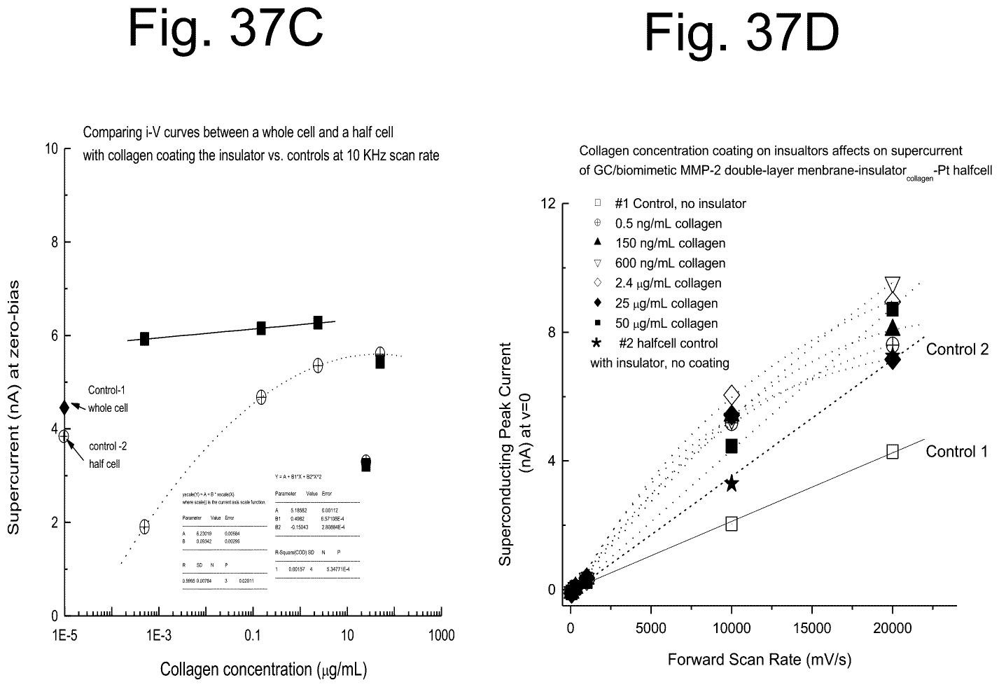

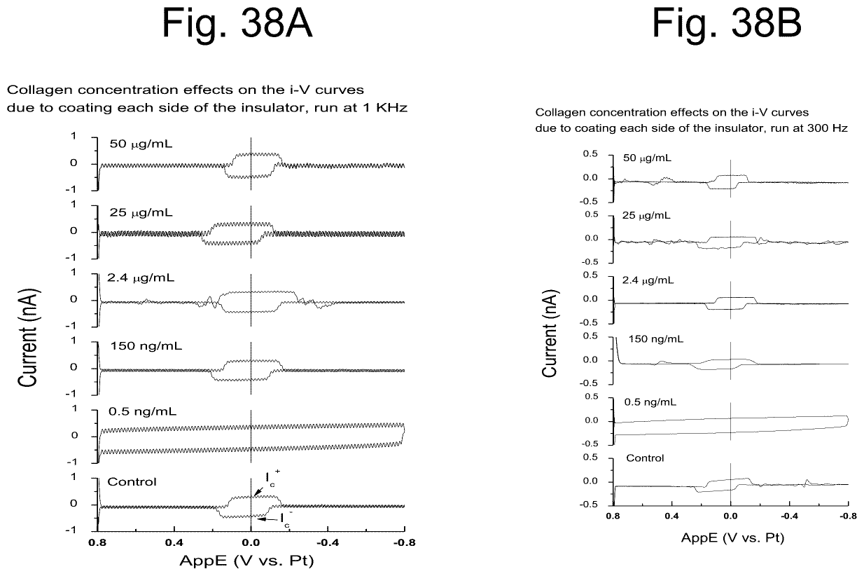

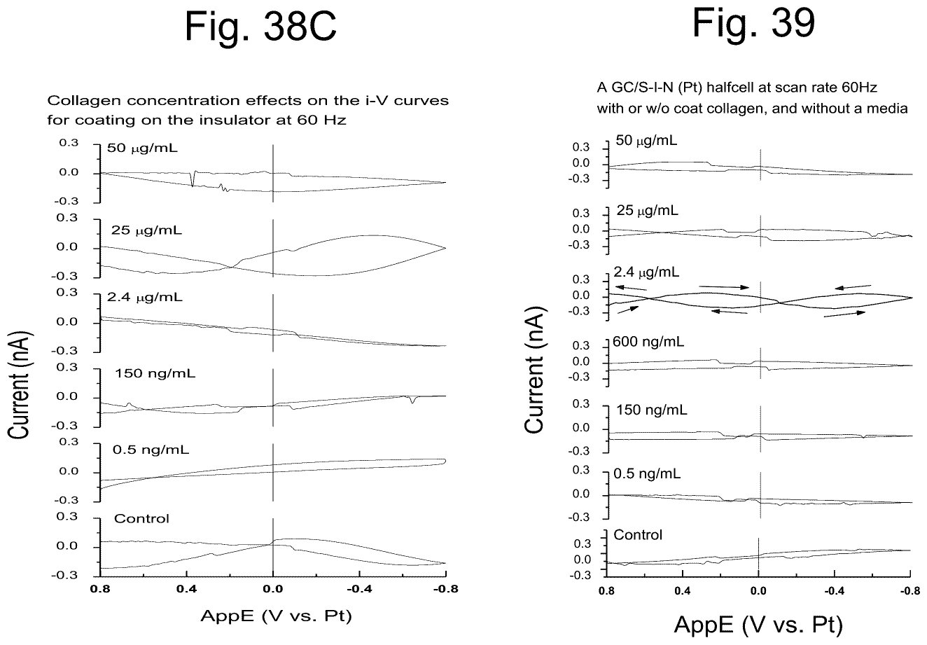

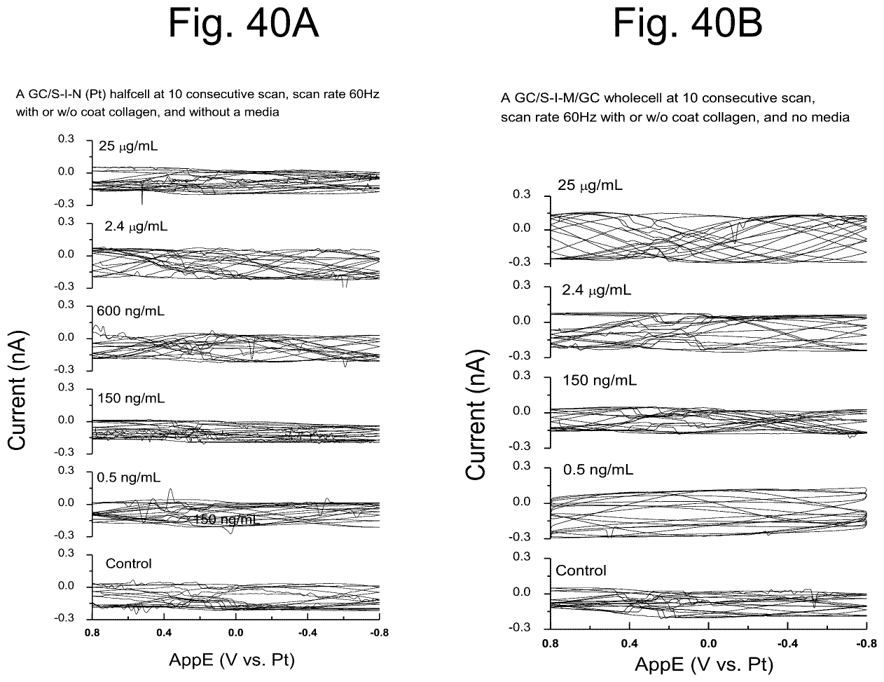

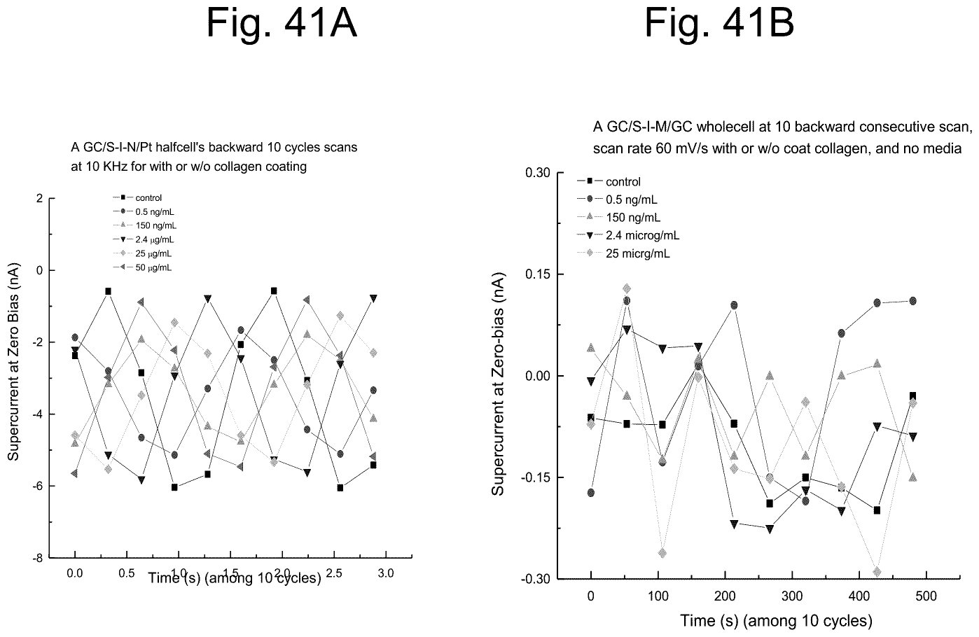

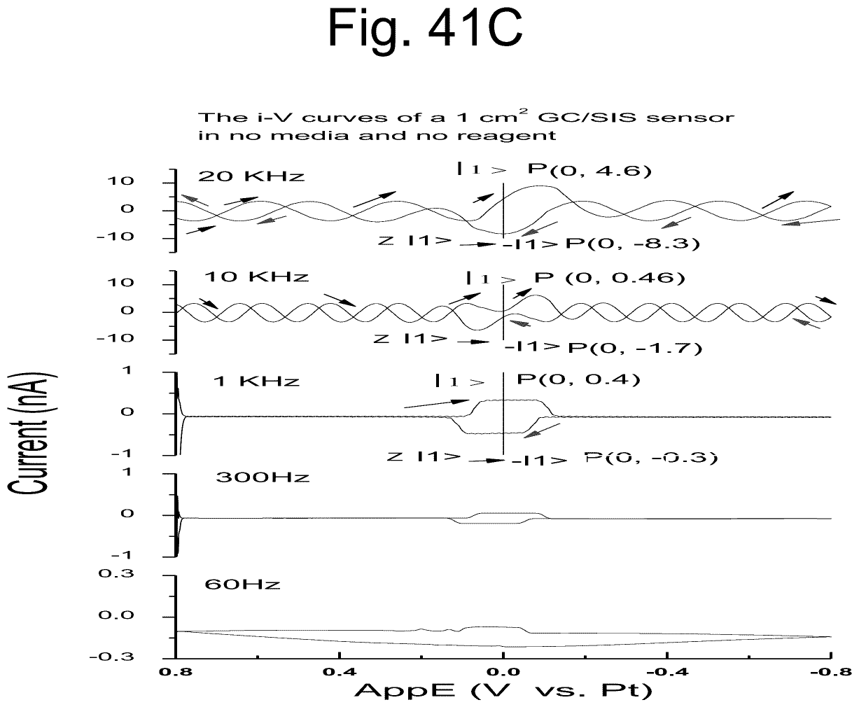

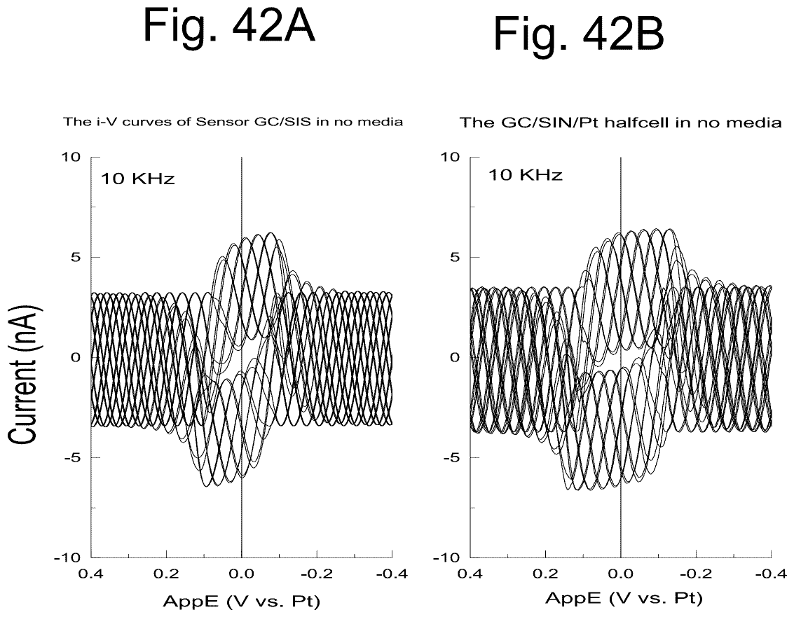

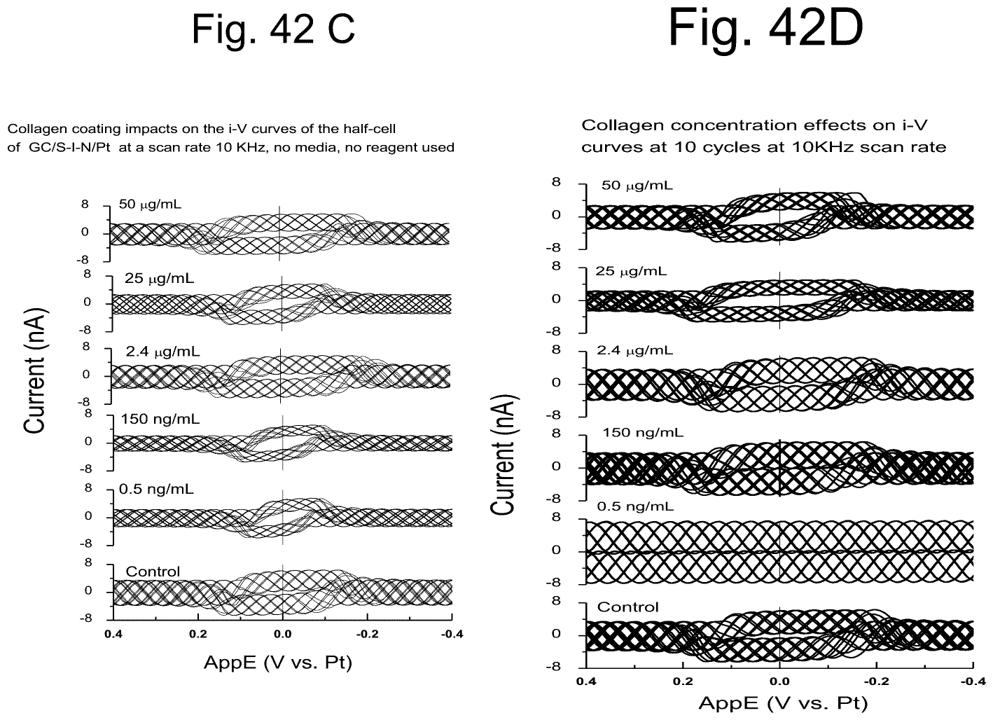

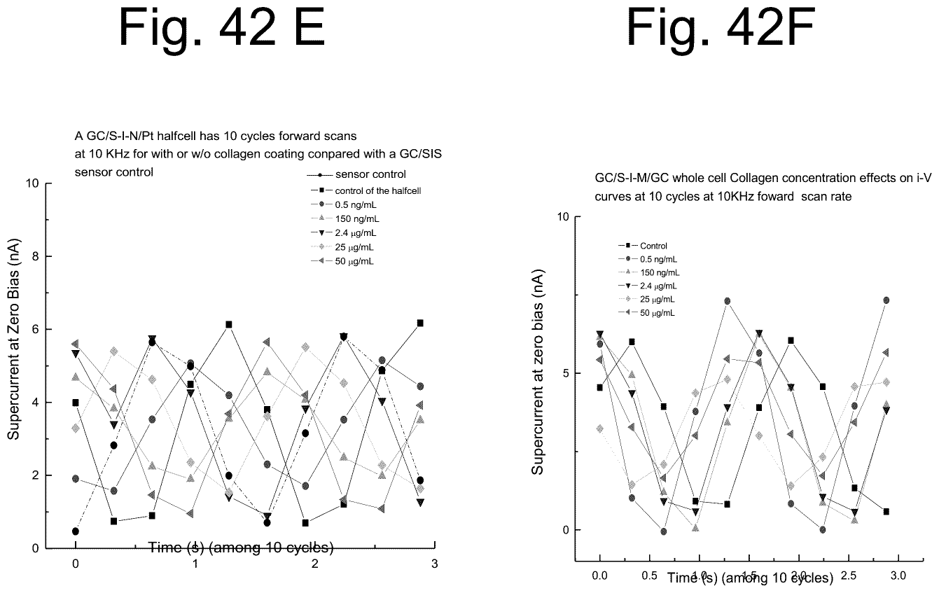

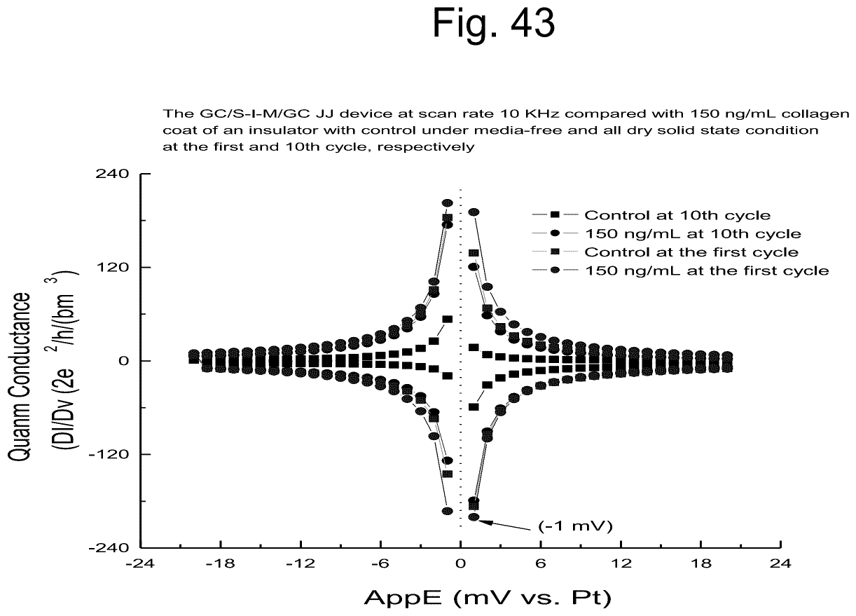

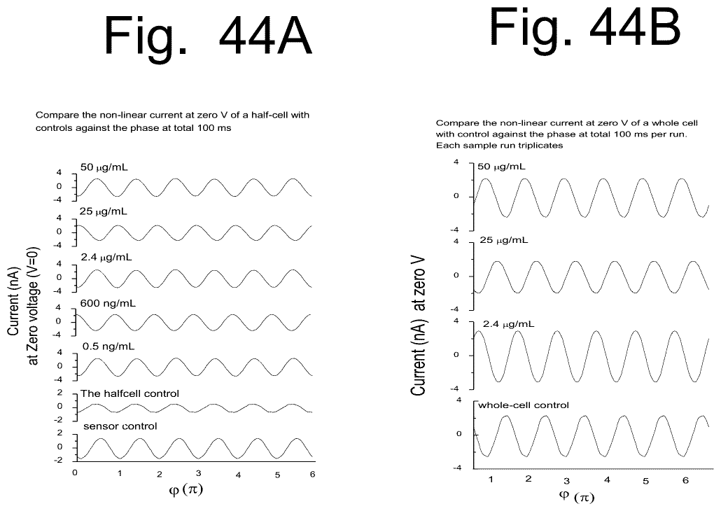

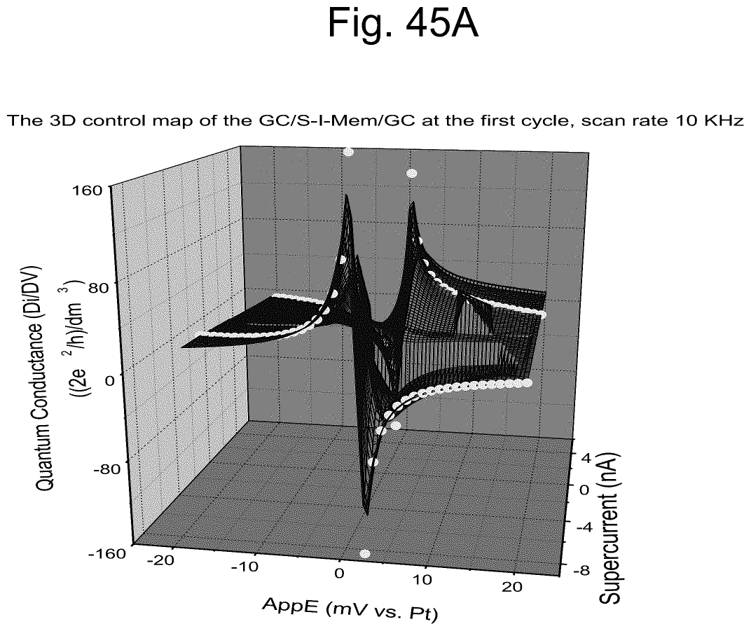

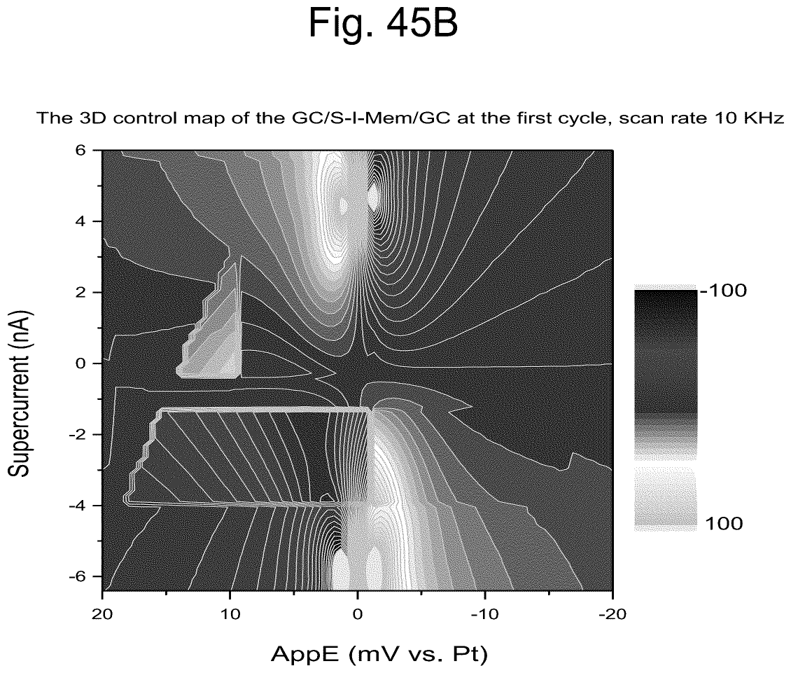

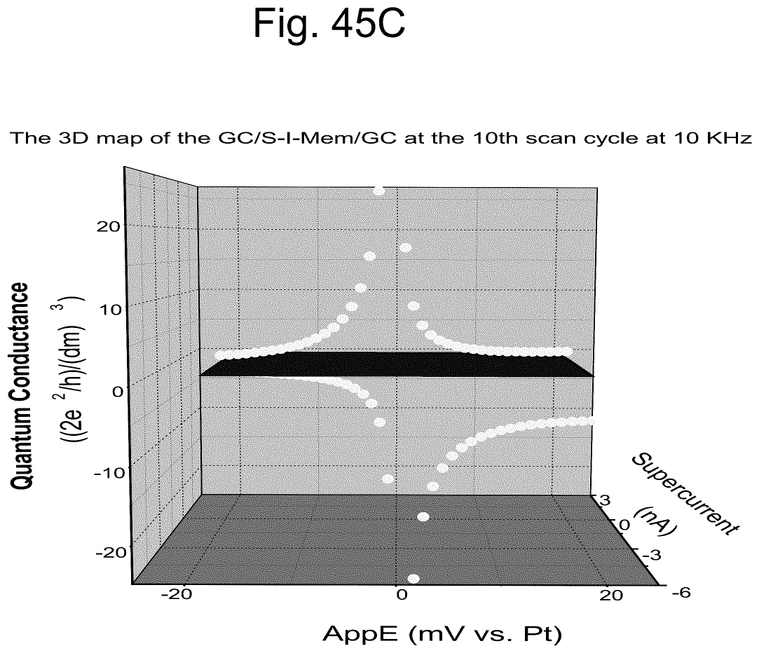

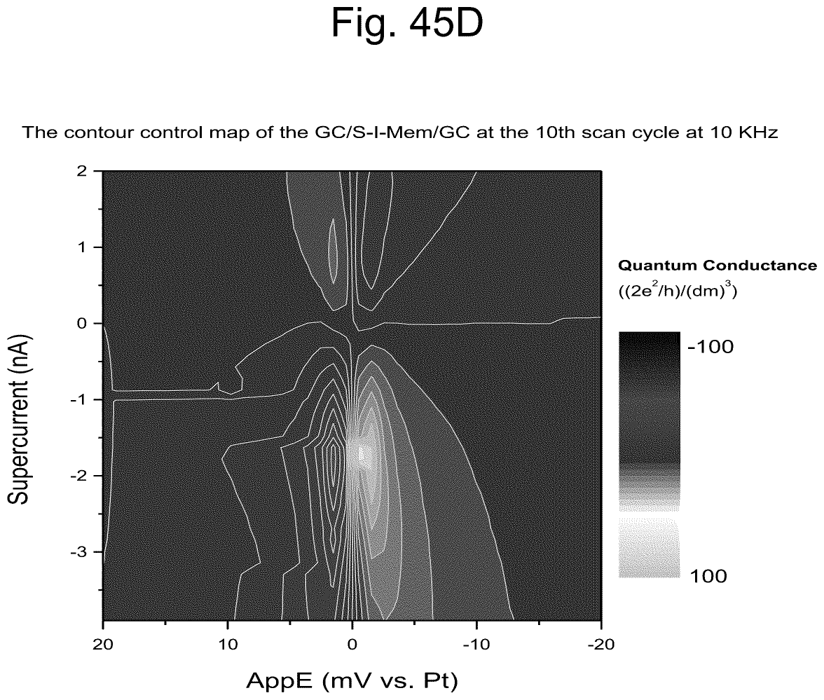

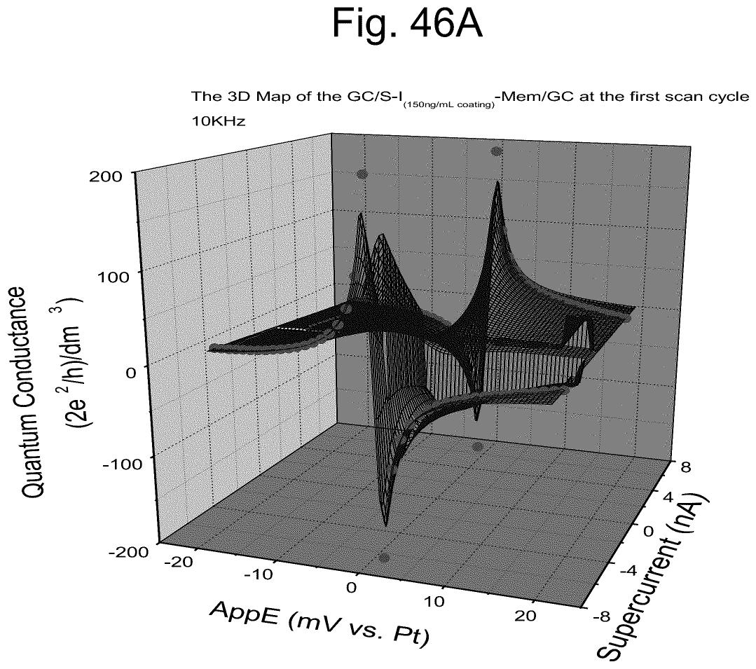

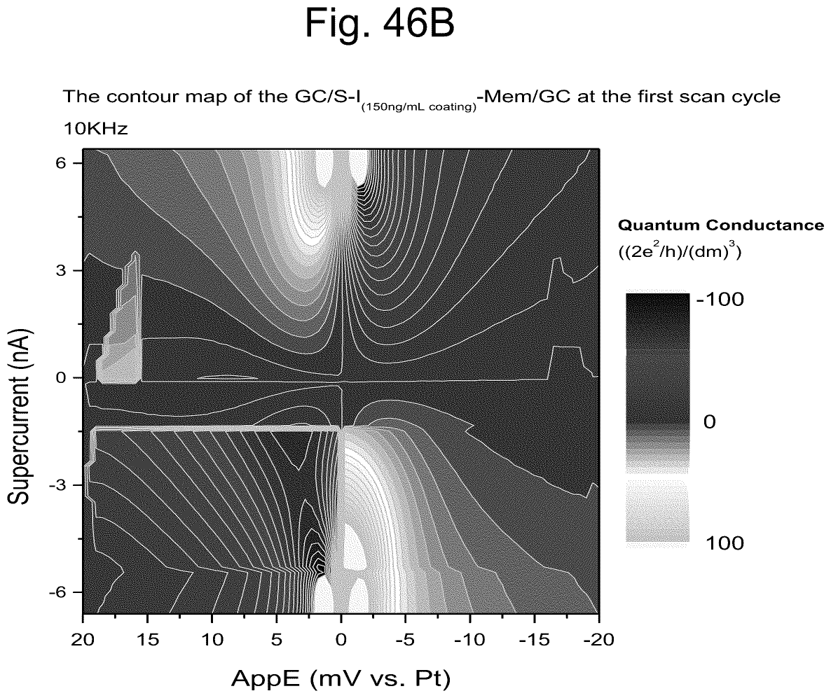

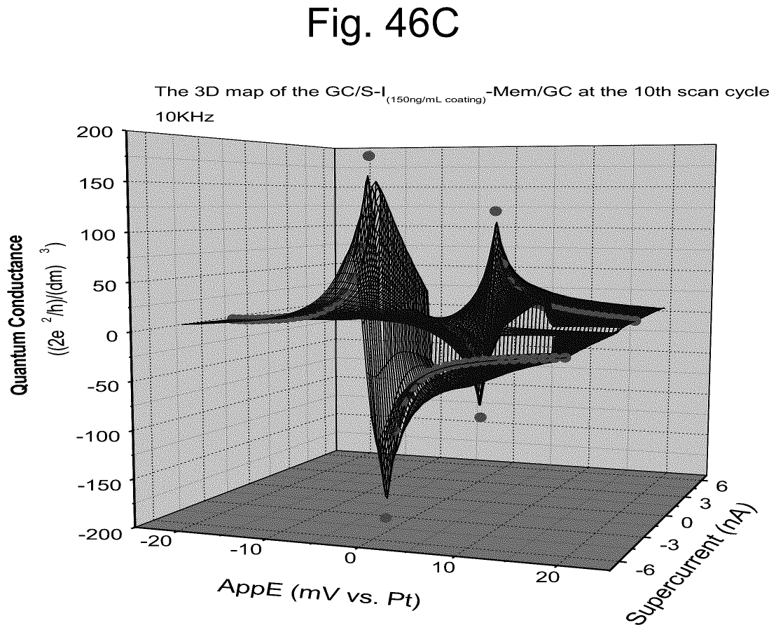

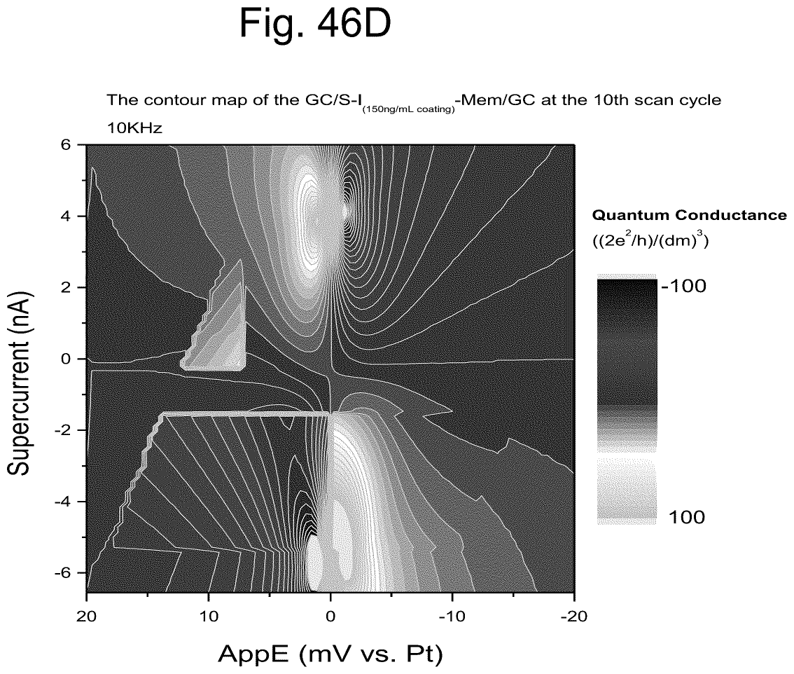

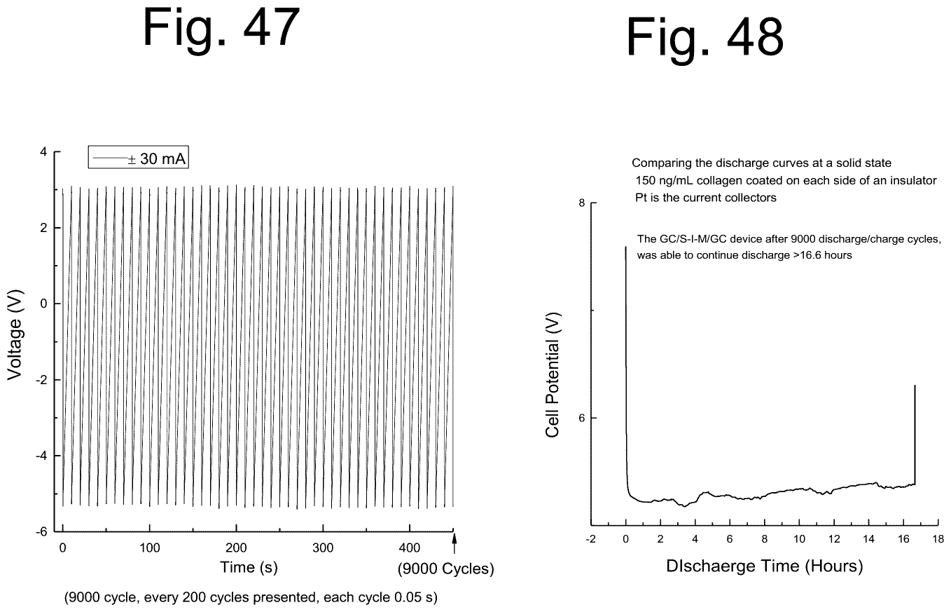

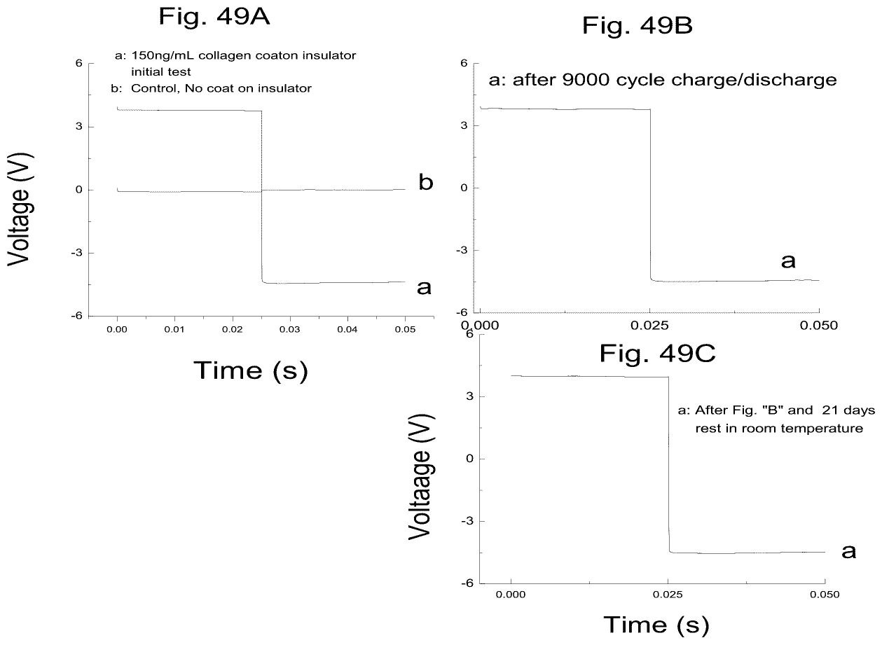

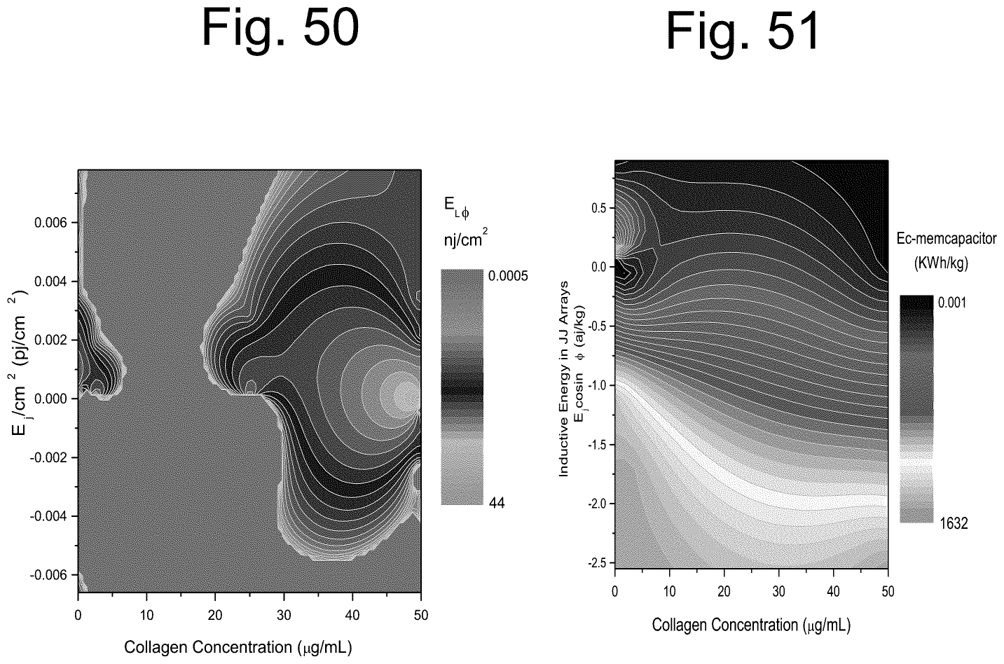

FIG. 4A illustrates the Direct Electron-relay (DER) art model made by a cage-structured polymer network between the zinc ions from the activated biomimetic MMP-2, by elimination of the cysteine group from the polymer mixture of bM-β-DMCD/TCD/PEG/PVP/ZnCl₂ before deposited on the gold chip, and the mM-β-DMCD (short names as MCD) in Tris/HCl buffer. Zinc ions coordinated with three imidazole groups (two from the bM-β-DMCD and one from the MCD), and either with the COO-of TCD, or OH-group from the cyclodextrin, or from water. Furthermore, all other hydrogen bonding, hydrophobic interaction, and the p-p interaction between the functional groups inside the cavity induced the DER force evidenced as the Cooper pair electron freely moving toward the same direction shown in FIG. 3D and the cylinder cage structure was shown from FIG. 3F to FIG. 3I.

FIG. 4B depicts the art model when ATP interacts with the cylinder polymer network molecules that form an enhanced DER. This effect has enabled ATP to play a role to transform a memristive/meminductive sensor to a superconductor at a scan rate ≥60 Hz with an ATP concentration higher than 400 aM at the zero-bias potential.

### EXAMPLE 4

### The JJ Characteristics

The hallmarks of the JJ characteristics are (1) at a DC voltage=0, a supercurrent

$${I}_{s} = {I}_{c} sin ( Δφ ) \qquad ( 1 )$$

I_{c} is critical current, Δφ is the phase difference between the waves of two superconductors, which appears at the DC Josephson junction; (2) at a finite DC voltage, the phase of the supercurrent has changed as a function of time that caused oscillating at the AC Josephson Junction, which is proportional to 2eV_{DC}, i.e.,

$$\partial φ / \partial t = ( 2 e / h ) {V}_{D C} . \qquad ( 2 ) [ 28 ]$$

Here the Planck constant=h/2π(1.055×10⁻³⁴ Js). Scan frequency affects i-V curves shown in FIG. 5A for Sensor 1 is different from that of Sensor 2 in FIG. 6, both figures were working in the Tris/mono-substituted imidazole dimethyl-β-cyclodextrin (mM-β-DMCD), in short, MCD control media over 1 Hz to 25 kHz. Sensor 1 demonstrated a memristive behavior with a hysteresis point at zero V and zero current at 60 Hz, but it had no superconductivity through all the curves, which indicates the heating procedure damaged the superlattice structure leading to a poor Cooper pair formation. In contrast, Sensor 2 demonstrated perfect memristive behaves at 60 Hz and also showed the superconductivity at zero-bias potential over 5 kHz to 25 kHz as shown in FIG. 6. This fact showed as long as the Cooper pair formation, the superconductivity can be expected. The sine waves oscillated in the high scan frequency are observed.

### EXAMPLE 5

### Superconductivity with Super-Positioning

Because Sensor 1 lacks Friedel-oscillation due to the heating process for eliminating the cysteine group, without any superconductivity regardless with or without ATP in the Tris media as shown in FIG. 5A (without ATP) and 5B (with ATP) over a wide range of scan rate. Scan frequency effects on the Shapiro step voltage in the sine waves of Sensor 2 over 5 kHz to 25 kHz, under external magnetic field=0 condition, was observed as shown in the i-V curves in FIG. 6, FIG. 7, and FIG. 8 for without and with ATP under 60 and 10 kHz scan rate, respectively. The superlattice quantum bit's superposition states were seen between state “1” at v=0, I>0; “−1” state at v=0, I<0 and state “0” at v=0, I=0 at zero bias in FIG. 7 and FIG. 8.

### EXAMPLE 7

### Sensing ATP by Sensor 2 Using the CV Method and ATP Promotes Super Conductivity

FIG. 5B depicts the Direct Electron Transfer (DETred) peak current increased exponentially for Sensor 1 as the ATP concentration increased from 40 fM to 60 nM compared with the control having 66.5, 146, 3048.7, and 4664-fold increase in four levels, respectively at 60 Hz scan rate. FIG. 6 shows scan rate impacts on Sensor 2's i-V curves over scan rate from 1 Hz to 25 kHz in the Tris/HCl/MCD control media.

FIG. 7 shows Sensor 2's sine or cosine wave peak superconducting current intensity increased or decreased exponentially for the forwarding scan and backward scan, respectively, over ATP concentrations from Panl B, 400 aM; Panel C: ATP 400 fM; Panel D: ATP 200 pM; Panel E: ATP 200 nM; Panel F: ATP 2 μM, at zero-bias at the same scan rate of 60 Hz compared with the controls in FIG. 6. We observed that a 25 aM ATP concentration is not enough to turn memristive to superconductivity shown in Panel A. The superposition characteristics were shown also as an example labeled in Panel F. The exponential increase of the superconducting current vs. ATP concentrations over 400 aM to 2 μM for the forward scan against the backward scan, which is in an exponential decay, were shown in FIG. 9. At a high scan rate of 10 kHz, both the forward and the backward scan's supercurrent at zero-bias increased non-linearly from 200 fM till near 1 μM, both currents is dropped drastically as shown in FIG. 10. FIG. 10 depicts the supercurrent vs. ATP concentration curves of the DET_{red} and DETₒₓ peaks at the zero-bias over ATP concentration ranges from 200 fM to 200 μM. Data was obtained according to the figures from FIGS. 8A to 8I.

### EXAMPLE 9

### ATP Induces Phase Change in Detail Presented in Sensor 2

Above Section we discovered ATP promoted superconductivity at an appropriate concentration and scan rate in the Tris buffer, now this Section we explain the invention of the technology on Sensor 2 and discovered the ATP's other role which is for inducing a phase change as shown in FIG. 11 for 10 consecutive scans at 60 Hz. The curves from Panel A are with an ATP concentration of 400 fM; Panel B with ATP 200 pM; Panel C with ATP 200 nM, and Panel D with ATP 2.0 μM, respectively. The phases of the i-V curves were constantly changing at a fixed scan rate, but the ATP concentration was increased.

FIG. 12A depicts the i-V curves of a 25 aM low concentration ATP in consecutive 5 scan cycles at a scan rate of 60 Hz compared with the control of Sensor 2. There was no phase change occurred, and the hysteresis curves are seen through the 5 consecutive scans. FIG. 12B depicts when concentration increased to 400 fM, in the consecutive 9 scan cycles of the same scan rate, we observed the cross-point moved away from the origin, and the i-V curves having the degree of superconductivity at zero-bias, and companies with a phase change occurred compared with the control. FIG. 12C also depicts a similar trend when ATP concentration increased to 200 nM ATP concentrations, Sensor 2 shows the phase changes and superconducting current.

### EXAMPLE 10

### The 3D Quantum Conducting Map in the Multiple-Variable Study

Quantum computing is computing using quantum-mechanical phenomena, such as superposition and entanglement [28]. Superconducting flux qubit has two states that can be effectively separated from the other states and is the basic building block of quantum computers. Current DC or RF superconducting SQUID are made in advance for a faster switch time; however, hundreds of MHz electromagnetic field applied onto a tank circuit coupled to the SQUID is needed for the system to work under cryogenic conditions [29-31]. The RF-SQUID consists of a superconducting ring of inductance L interrupted by a JJ, the potential energy of the SQUID and the Hamiltonian equations are given by

$$U ( Φ ) = {( Φ - {Φ}_{e} )}^{2} / 2 L - {E}_{J} cos ( 2 πΦ / {Φ}_{0} ) \qquad ( 3 ) [ 28 ]$$

$$H = {Q}^{2} / 2 C + {( Φ - {Φ}_{e} )}^{2} / 2 L - {E}_{J} cos ( 2 πΦ / {Φ}_{0} ) \qquad ( 4 ) [ 28 ]$$

Φₑ is the applied magnetic flux penetrating the SQUID ring. Φ is the total magnetic flux threading the SQUID ring, L is the inductance, E_{J} represents the Josephson coupling energy, and Φ₀ is the superconducting magnetic flux quantum, Q is the charge on junction's shunt capacitance satisfying [Φ, Q]=ih/2π, while h is the Planck constant.

Stern's group reported the observation of Majorana bound states of Josephson vortices in topological superconductors, and the equations of three types of energy contributions to the Josephson vortices in a long circular junction in a Sine-Gordon system was published [32]. The Josephson junction energy was from the Cooper pair, the magnetic energy was from the inductivity of the circular vortex, and the charge energy was from the SIS quantum capacitor-like device [32-33]. Our group reported using a 3D dynamic map method, to elucidate the multiple-variables between the component energies contributing to the superconductivity of the vortex array system at room temperature without external magnetic field applied. Our experimental data were shown on the i-V curves and the AFM structure of the superlattice array. The modified Sine-Gordon system energy for our d-wave vortex array is:

$${E}_{JJA}^{n} = ( 1 / 2 ) {{C}_{i}^{- 1} ( Q - e {n}_{1 … i} )}^{2} \qquad ( 5 )$$

$${E}_{L}^{n} = ( 1 / 2 ) {μ}_{0} {N}_{n = 1 ¨ i}^{2} . A . {L}_{n = 1 .. i}^{- 1} . {I}_{n = 1 . . i}^{2} \qquad ( 6 )$$

where Eⁿ_{jjA} is the charge energy of Josephson Junction arrays at n=1 . . . i; Q is the charge, C is the total capacitance at n=1 . . . i, en is the n quantum particles at 1 . . . i data point with energy periodic in h/e for Josephson effect for d-wave [34]; Eⁿ_{L} is the Inductive energy induced by the circular toroidal array. N is the turning number around the toroidal porous at n=1 . . . i, A is the cross-sectional area of the porous, L is the length of the wending, μ₀ is the magnetic permeability constant in free space; I is current. The toroidal arrays are in series connected. A recent publication regarding our FFTJJ multiple-variable study results in 3D dynamic maps was presented in the literature [34]. In this invention, the multiple variables, such as the ATP concentration and the applied potential effect on the quantum conductance were studied through the 3D mapping method without decomposing the superconducting energy into

several components. [00060] FIG. 13 depicts the 3D dynamic map of the relationships over 5-level ATP concentrations from 0.2 pM to 2.0 μM over the potential range between −40 mV to +40 mV to illustrate the relationship among ATP concentrations, zero-bias potential, and the differential quantum conductance using 10 kHz scan rate forwarding scan data. From the map, the quantum conductance values are correlating with the ATP concentrations at zero bias, except at a higher concentration near 1.0 μμM, it was dropped.

### EXAMPLE 11

### Quantitation of ATP in Biological Specimens

### The Chronoamperometric Method (CA) was Measured by Sensor 1

FIG. 14 depicts Sensor 1's current vs. time over ATP concentrations over 100 aM to 400 nM compared with the control in pH 7.8 Tris/HCl/MCD solution. FIG. 15 depicts the lower-end concentration curves. Inserts are enlarged views for lower end concentration compared with controls. Inserts are the enlarged view of the curves at the lower concentration compared with the control. FIG. 16 in Panel A depicts the regression calibration curve of current density vs. ATP concentrations in a logarithm scale for the x-axe and the y-axe (9 levels, n=27 over 100 aM to 400 nM) with the regression equation (y log scale) Y=1.9+0.57*(log scale)χ. r=0.996. S_{Y/χ}=0.16, p<0.0001. In the inversion of the equation. 0.57 is the power of the x, and 1.9 is the intercept from the log-log plot. Hence the inversed equation is F(x)=79x^{0.57}. FIG. 16 Panel B depicts Sensor 1's calibration curve over a linear range of 400 fM to 170 nM (7 levels, n=21) with the regression equation y=65.4+13.4x, r=0.997. Sy/x=65. p<0.0001. The Detection of Limits (DOL) of 0.56 fM over the analytical range 100 aM-400 nM with a relative Pooled Standard Deviation (RPSD) value of 0.9% (n=30).

### EXAMPLE 12

### The Open Circuit Potential Method (OPO)

Sensor 2's strong superconductivity has enabled the device to direct real-time monitor energy change under open circuit potential, under a reagent-free, antibody-free condition. FIG. 17 depicts the voltage curves exponentially increasing as the ATP concentration increase compared with the control in the buffer media. FIG. 18 depicts the non-linear calibration curve of voltage vs. concentrations over the range of 25 aM to 400 pM. FIG. 19 shows a plot for high-end ATP concentration with spontaneous voltage curves as the time (600s) over 0.8 nM (a) to 2 μM (d). FIG. 20 shows the calibration curve over the higher ATP concentration range from 0.8 nM to 2 μM.

### EXAMPLE 13

### The Double Step Chronopotentialmetry Method (DSCPO) by Sensor 2

The ATP concentrations also can be detected in several seconds using the DSCPO method and setting the fixed current as ±10 nA, and each step 4s with a data rate of 1 kHz. FIG. 21 depicts Sensor 2's voltage vs. time (each step 4s) curves by the Double-step Chronopotentialmetry (DSCPO) method in the presence of various ATP concentrations from 25 aM to 400 nM in the Tris/HCl/MCD buffer compared with the control. Each sample run triplicates. The curves show a positive correlation between the voltage intensity and the ATP concentrations in 7 levels compared with the lowest voltage from the control. FIG. 22A depicts the calibration curve of the normalized action potential divided by the mean signal vs. ATP concentrations over 100 aM to 200 nM. The relative pooled standard deviation (RPSD) is 0.24%. The linear semi-log plot gave an equation of yscale(Y)=A+B*xscale(X) with A=0.37 and B=0.06. r=0.992, n=18. S_{y/logx}=0.027, p<0.0001. The DOL found from this plot is 46 aM.

FIG. 22B depicts the low-end ATP calibration curve from ATP 25 aM to 400 aM in the Action potential results of the DER peaks after subtracting the background signals with an RPSD error of 1.3% with the signal increase rate of 0.016V/aM. The DOL result is 2 aM. FIG. 22C depicts the_semi-log plot of the absolute normalized resting potential vs. ATP concentrations under the same experimental conditions as FIG. 22A having a power 4.39 indicated the resting potential effects on ATP concentration lower than 100 aM, which is dominate than that of the action potential effect.

### EXAMPLE 14

### Accuracy and Imprecision

The USDA-certified organic milk for infants was compared with human milk (Lee Biosolutions. MO) without prior sample preparation. Human milk was collected from normal subjects who breastfeed 1 month-old newborn, each sample run triplicates.

Methods validations were studied through the recovery experiments using fresh human milk and USDA-certified organic milk for infants as controls compared with spiked 100 aM and 60 nM ATP and with or without low and high-level LPS challenges by the OPO method against each one's standards and the controls. The accuracy recoveries were 94±0.14% and 91±0.16% at 100 aM and 60 nM ATP for the human milk samples compared with organic milk samples of 85±0.2% and 79%±0.2%, respectively using the OPO method is traced back to the Tris standard control. The human mike control and the organic milk control samples have an agreement related to the Tris/HCl/MCD buffer sample controls (each type sample run triplicates) are 94.0±0.14% and 95.5±0.2%. respectively. The 5% difference is believed due to the artificial chaperone cage effect on the proteins of the milk. which is to lead the landscape free energy down to the lowest for a right folding [29-30]. After correcting this effect, human milk's recovery in the two levels' ATP challenges is 98±0.14% and 95±0.16%; the organic milk recoveries are 90.5±0.2% and 84.5±0.2%, respectively. The data implies the chaperoning effect has more impact on organic milk than that on human milk. In the two levels' LPS challenges (100 ag/mL. 60 ng/ml), the recovery results are 96±0.16% and 105±0.14% vs. 105.5±0.28% and 95±0.19% for human milk and organic milk, respectively.

Point accuracy and imprecision were studied through the recovery experiments using spiked human milk and the USDA-certified organic milk samples against the control samples with 2 levels of ATP concentrations at 100 aM and 60 nM, respectively. We compared the measured results with the calibration curve after subtraction of the voltage values from control samples using sensor 2 as shown in FIG. 22D of Table 1. We also studied the LPS effects on the recovery at 10 fg/mL and 90 fg/mL. respectively under a fixed ATP concentration of 60 nM. The results show the recoveries using human milk and organic milk samples with the voltage method are higher than 96% with an imprecision error less than or equal to 2% at the 100 aM and 60 nM levels ATP compared with the controls. respectively without LPS challenge. Using two levels of LPS challenges with the fixed ATP 60 nM, the recoveries are 103±0.8%, and 103±0.7% for human milk samples compared with the spiked controls at the same level in the buffer; using organic milk samples, the recoveries are 97±1%, 18.8±0.3% at 10 fg/mL LPS and 90fg/mL LPS, respectively traced back to the spiked controls at the same level in the buffer. These results showed organic cow milk samples are vulnerable to the LPS attack at higher-level 90 fg/mL, which caused an unacceptable result in recovery, but the human milk samples demonstrated immunological advantage with 100% recovery with two levels of LPS challenges even under 60 nM ATP concentration.

The CA method was also used to access the accuracy and imprecision. Human milk control samples against the standard control samples in the Tris/HCl/MCD buffer found no specimen interference having 101+6% traceable to the standards; In the presence of 30 fM ATP spiked in the human milk samples, i.c., the final ATP concentration is 30 fM in the sample, the recovery results are 100.0±7%; However, when 60 nM ATP presents in the human milk samples, due to the immunological property, human milk samples eliminated all the ATP's effect, led to no signals were measurable; In the presence of 30 fM ATP, under a 10 fg/mL LPS challenge of the human milk samples, the recovery results are 100±10%; Under 30 fM ATP, using a 90 fg/mL LPS challenge, the human milk samples produce 3-fold high signal intensity,

For comparison, the organic milk control samples were found to have a 21% of HSP 60-like chaperone interference for trace back to the standard control samples with 79±2.6%; in the Tris/HCl/MCD buffer; After correcting the interference, in the presence of 30 fM ATP spiked in the organic milk samples, the recovery results are 100.0±4.4%; when 60 nM ATP presents in the organic milk samples, the recovery results are 102.3±0.02%; In the presence of 30 fM ATP, under a 10 fg/mL LPS challenge of the organic samples, the recovery results are 49±0.2%; Using a 90 fg/mL LPS challenge to the organic milk samples, the recovery results are 130 +3.6%.

### EXAMPLE 15

### Applications for Defining the Transformational Immunomodulant Between the Anti-Inflammatory and the Pro-Inflammatory Status

We primarily suggest the turning point of the ATP concentration from anti-inflammatory to pro-inflammatory for a “healthy” Biomimetic MMP-2/HSP60 sensor 2 in the extracellular environment is higher than 800 nM. FIG. 23 further shows a contour map relationship between the ATP concentration (as the y-axis), the applied potential (as the x-axis), and the quantum conductance (as Z-axis). It was observed that the range between the highest quantum conductance values at zero bias is associated with the ATP concentration between 200 fM to 800 nM, so we define this range as having the Physiological High-Frequency Oscillation (Phy-HFO) as the “Anti-inflammatory” range; when concentration higher than 800 nM, the quantum conductance values are reduced, and this range was defined as the “Pro-inflammatory” in the extracellular ATP concentration range at 10 KHz. Because Sensor 1 can be viewed as a “Mutated” or “Stressful” Biomimetic MMP-2/HSP60 model as far as the capability to promote the Cooper-pair electrons' concerns, was greatly diminished as shown in the AFM image FIG. 2B, hence it was shown Sensor 1 neither have superconducting peaks in the presence of ATP in the buffer media in 60 Hz and 10 kHz scan rate, respectively.

### EXAMPLE 16

### Applications in Defining ATP Concentration Ranges Effecting on Cell Reversible Membrane Potential (RMP) and Its Ratio Between Action Potential and Resting Potential

It is a well-recognized phenomenon that cancer cells have abnormal cell membrane potential [35-38]. Biologists measure cell membrane action and resting potentials with burdensome instrumentation and time-

consuming procedures. A recent report shows breast cancer cell division caused a membrane potential increase due to variations in ion channel expression. Because the normal cell membrane action potential is 58 mV, and −70 mV is for the resting potential [39], the small signals are very easily buried in the background noises that can cause problems for pediatric neurologists and intensive care unit doctors who need strong signals to monitor and diagnose the neonatal neurological diseases [40]. The consequences of human cancers, trauma brain injury (TBI) and other diseases are not being able to maintain mitochondrial cells' reversible membrane potential (RMP) and unable to maintain the normal membrane's potential ratio between the action potential and the resting potentials [35-44]. Our group first used the biological marker of the action/resting potential ratio to monitor the treatment of triple-negative breast cancer and the brain cancer prognosis in a 3D heat release map [41-44]. There are very few, if any, use the ratio of action/resting potential as a biomarker to monitor and define the ATP concentration ranges, that transform from anti-inflammation to pro-inflammation in the presence of bacterial toxins under a well-controlled system without any sample processing, no labeling and no tracers were used. FIG. 24 shows the cell RMP voltage (after subtracting the control) is not depending upon the ATP concentrations between the range from 100 aM to 800 nM with a power of 0.013, which means the cell voltage increase rate is only 1.3% of the ATP concentration increase rate, hence this range is the “safe zone” confirmed by FIG. 10 using the 3D map method based on the CV data. However, we found concentrations lower than 100 aM and higher than 800 nM the cell RMP cannot keep constant, either lower than the normal average cell voltage, or 10, 20-fold higher than the normal average cell voltage, therefore, we define this range as the pro-inflammatory toxin range, for contrast, the “safe zone” range was defined as the anti-inflammatory toxin range.

FIG. 25 further shows the extracellular ATP concentration range effects on the ratio of the cell action potential vs. resting potential. The results showed a concentration lower than 100 aM or higher than 400 nM will lead to a ratio of action vs. resting potential either significantly lower or significantly higher than the normal ratio range, which is between 0.7 to around 1.0 [45-50]. Our prior works revealed that living cancer cells can lead to an abnormal ratio up to 3. 10 to 100-fold higher than this range according to the nature of cancer and the stage of the cancer are in. Even FIG. 24 reveals the total cell voltage at 800 nM is in the “safe zone” with respect to the energy concerns, but clinically, the ratio value in FIG. 25 further revealed the 800 nM ATP concentration led to a 10-fold higher than the normal ratio range, hence in FIG. 25 we excluded the 800 nM points. The ratio vs. concentration curve has a power of 0.08 which means the ratio increase rate is 8% of the concentration rate increase having a CV value±4.5% error in the normal range was demonstrated.

### EXAMPLE 17

### Applications in Assessing Human Milk Immunological Advantage in Preventing Extracellular ATP Hydrolyzation

Assessing human milk's immunological advantage in preventing extracellular ATP hydrolyzation is important. Our study was conducted through the CV method using Sensor 1 because we knew Sensor 1 lacks Copper-pair electrons, and we used the human milk samples compared with the certified organic milk samples for infants under the same experimental conditions. FIG. 26, Left Panel shows the extracellular hydrolysis DETₒₓ curves (also called the memristive peak) happened in the presence of spiked 60 nM ATP (final concentration), which caused a 9-fold increase of the peak current at 700 mV compared with the organic milk negative control. There is an unknown peak observed in the organic control milk sample located at 20 mV, and the peak was the 2.8-fold increase of the amplitude when ATP presences. In contrast,

FIG. 26 in the Right Panel shows the buffer control sample of the CV curve has no such unknown peak at 20 mV. There is a current increase for the DETₒₓ peak at 700 mV of more than 173-fold in the presence of 60 nM ATP compared with the control, and the DET_{red} peak intensity also increased 150-fold in the presence of ATP compared with the control buffer sample.

The DETₒₓ has a first-order ATP hydrolysis rate of 5.92×10⁻³/s by plotting the peak currents vs. scan cycles (each cycle is 53.33 s) as shown in FIG. 27. The top Panel curve is for the DET_{red} peak current vs. scan cycles; the bottom Panel curve is for the DETₒₓ peak current vs. scan cycles.

Human milk samples communicated with the “mutated” HSP60 membrane on the same Sensor 1 having a different manner compared with the organic cow milk samples. FIG. 28A, FIG. 28B, FIG. 28C and FIG. 28D depict the human milk samples under the impact of 60 nM ATP concentration in 4 scan cycles (2 more cycles curves did not show) compared with the human milk controls using Sensor 1 at 60 Hz. The nodes, the super-positioning, and the phase change at the zero-bias were observed, and there was no ATP hydrolysis signature peak observed, and there was no unknown DET reduction peak noticed. FIG. 29 shows an alternative up and down peak superconducting current vs. scan cycles for the forward and backward scan, respectively. The human milk samples have “eyes” that can see the danger and purposely avoiding communicate with the ATP molecules because the human milk contains living microbiota, in which the HSP60 and ZnT chaperonin proteins in the extracellular play a role in the guardian's protection. FIG. 30 shows the overlapping curves of the control human milk sample in 6 consecutive scans vs. the milk sample with 60 nM ATP. The peak magnitude kept the same, but the phase change is constantly happening with or without ATP, that indicates human milk promotes an orderly electromagnetic energy stored in the cell for enhancing the brain development and memory caused by the mem inductivity through the Josephson junctions, in which are advantages compared with the “chaos” electromagnetic energy acquired from Alzheimer's β-amyloidal accumulation [51-54].

### EXAMPLE 18

### Experimental

Sensor 1 has an activated biomimetic MMP-2 membrane by a heating method at 80° C. for 5 minutes using the innate biomimetic MMP-2 membrane fabricated based on a published procedure [34]. Sensor 2 was also in a state of activation of biomimetic MMP-2 by a direct deposited method with compositions of TCD, PEG, PVP, bM-β-DMCD, and embedded zinc chloride on gold chips with appropriate proportions at 37° C. for 96 hours. The USDA-certified organic milk for infants was compared with human milk (Lee Biosolutions, MO) without prior sample preparation. Human milk was collected from normal subjects who breastfeed 1-month-old newborns, and each sample run triplicates.

The morphology of the AU/SAM was characterized using an Atomic Force Microscope (AFM) (model Dimension Edge AFM, Bruker, MA). Data was collected in TappingMode using silicon probes with a 5-10 nm tip radius and ˜300 kHz resonance frequency (Probe mode TESPA-V2, Bruker, MA).

### EXAMPLE 19

### Conclusions and Discussions

A multiple-functioning superconductive device was invented based on Toroidal Josephson Junction (FFTJJ) array with a 3D-cage structure self-assembled organo-metallic superlattice membrane. The device not only mimics the structure and function of an activated Matrix Metalloproteinase-2 (MMP-2) protein, but also mimics the cylinder structure of the Heat Shock Protein (HSP60) protein, that works at room temperature under a normal atmosphere, and without external electromagnetic power applied. The device enabled direct rapid real-time monitoring of atto-molarity concentration ATP in biological specimens and was able to define the anti-inflammatory and pro-inflammatory status revealing a transitional range of ATP concentration under antibody-free, tracer-free, and label-free conditions.

### Followings are the Specifications in CIP Application

### DETAILED DESCRIPTION OF THE INVENTION

### Example 1

### Fabrication of the Nanobiomimetic Organometallic Superconductor/Mem-element Qubit Devices with Superlattice Structures

The double-layer membrane comprised of a layer of bis-substituted imidazole-β-dimethyl cyclodextrin (bM-β-DMCD), triacetyl-β-cyclodextrin (TCD), polyethylene glycol diglycidyl ether (PEG) and poly (4-vinylpyridine) (PVP), zinc chloride with an appropriate composition deposited on the first layer of 1 cm₂ GC/TCD/PEG/PVP/CD copolymer was fabricated by a self-assembly method using a gold chip as the substrate [9-11]. The morphology of the GC/SAM was characterized using an Atomic Force Microscope (AFM) (model Dimension Edge AFM, Bruker, MA) [9-11]. Another GC/MEA comprised TCD/PEG/PVP/CD copolymer with o-nitrophenyl acetate (NPA) cross-linked organic conductive Battcell membrane cited in literature on GC electrode procedures were followed [27-30].

Coating the dielectric insulator procedures comprised of depositing 60 μL collagen in pH 7.4 PBS solution on each side of the insulator in a chosen concentration level between 0.5 ng/ml to 50 μg/mL, and putting the coated insulator into the incubator for drying of 2 hours at 40° C. for each side, until two sides are dried. The entire cyclic voltammetry (CV) measurements, the Double-step Chronopotentiometry (DSCPO) measurements and the single-step Chronopotentiometry (CPO) measurements were conducted under all solid, media-free, and reagents-free conditions at room temperature.

The 1 cm² cells were assembled with a GC/double-layer superconductive-I_{collagen}-M/GC configuration with each of the two platinum (Pt) wires contacted with the each of the two GC/MEA separated by the insulator and fasted compared with the control cell without coating collagen-1 on the insulator. The Pt current collectors are connected with the adaptor cables, then connected with the EC working station. The GC plate is 2 mm in thickness. Data was acquired by the electrochemical work station Epsilon (BASi Corp. West Lafayette. IN 47906), and data were processed by BASi's software package. Final statistic data analysis and 3D maps were conducted by the software package OriginPro 2022 (Originlab. Northampton, MA 01060).

Fabrication of a “Control” half-cell for comparison purpose with the whole cell of GC/S-I-M/GC was implemented by using the 1 cm2 GC/S-I-N/Pt configuration: the anode GC/MEA comprised GC/double-layer superconductive nanopore membrane, which the embedded zinc ions serve for the secondary Josephson junction toroidal array, and the GC/membrane contacted with a Pt wire, then both contacted with a collagen coated dielectric insulator, then fasted the half-cell as the working electrode assembly connected with the anode lead of the adaptor cable; another Pt wire serves as the cathode electrode connected with the cathode lead in the adaptor, and the reference lead also connected with the bare Pt wire for reference. All measurements were done without a media, and no reagent used.

Fabrication of a “Control” sensor for the purpose of comparison is fabricated as same as above paragraph except without the coated insulator, the GC/double-layer superconductive nanopore membrane directly contacted with the Pt wire fasted connecting with the anode lead of the adaptor cable, and another Pt wire connected with the cathode lead, and all others are same as in the above paragraph.

### Example 2

### The Friedel-Oscillation in the Superlattice Membranes

Friedel-oscillation is a phenomenon of Cooper-pair long-range indirect interactions between electrons

on the superlattice of the superconductive surface [16]. FIG. 31A depicts the AFM image of the double-layer membrane on a 1 cm² glassy carbon (GC) electrode in a 1.2×1.2 μm² AFM image. The top layer comprises bis-substituted imidazole-β-dimethyl cyclodextrin (bM-β-DMCD), triacetyl-β-cyclodextrin (TCD), polyethylene glycol diglycidyl ether (PEG), poly (4-vinylpyridine) (PVP), and zinc chloride with an appropriate composition deposited on the bottom layer of 1 cm² GC/TCD/PEG/PVP/CD copolymer. It comprises an elongated shine zinc cluster in Friedel-oscillation sited on superlattices on a curvature “bridge”, underneath there can be seen a pore. FIG. 31B depicts a 507.1×507.1 nm² AFM image in high sensor mode comprising many orderly superlattices with zinc clusters having shine Friedel-oscillation which demonstrates the JJ toroidal array. Further, FIG. 31C depicts a larger 2.5×2.5 μm² 3D AFM image with the zinc cluster's tower structure as the Josephson junction that promotes Cooper pair tunneling along the superlattices. The zinc clusters acted as an “insulator” or “bridge” in the superlattice GC substrate serves as a part of the superconductor-insulator-superconductor (S-I-S) configuration, and also serves as the anode metal electrode assembling (MEA). We reported in our prior publications that the same compositions of the membranes with the same propositions of cach component fabricated on a gold substrate observed the Friedel-oscillation [7-11]. FIG. 31D depicts the 2D view AFM image of the 16.8×16.8 μm² nano porous structured surface. FIG. 31E shows the AFM image results for surface roughness analysis over the larger area having a R_{q} 20.2 nm against 283 μm². FIG. 31F depicts the Section analysis results over the larger porous area. which appears several “Five-petal Starflower” shapes with three measurements having pore depth 4.56 nm and an outer diameter 4.8 μμm. FIG. 31G depicts the Section analysis results over the bright small porous area with three measurements for the “Five-petal-Starflower” (Pentas flower) having a star-like pore depth 6.46 nm and a diameter 931 nm with a Rms 14.2 nm among the 13.7 μm². The zinc clusters are brightening the star on the petals as the “pearls” or “morning dew” as the key insulating element for JJ array GC/S-I-S.

FIG. 32A depicts the 8.4×4 μm² AFM image of the detailed toroidal structure with five separate petals. and the zinc ion clusters played a role of an “insulator in the middle of the Josephson junctions or as a “bridge” connect the petals having a barriers' JJ width between 107.7 to 180 nm and a length between 0.89 μm to 1.4 μm. It was noticed that the “Five-petal Starflower” has a multiple-layer structure with multiple small starflowers inside of the large cavity, that has a JJ barrier's width between 53 nm to 72 nm having a length 180 nm to 360 nm. FIG. 32B depicts a detail view of the “fly bee” in FIG. 32A, may be come from a zinc cluster shaped like a “bee” flying on the layered starflower petals in the 3.2×3.2 μm² image, or it simply is the Cooper-pair as the “fly-bee”, because in FIG. 31D in the center of the bottom image we see the five-petal starflower with the “fly bee” on the right-hand side. and there is no any fly bee on the left-hand side of this starflower, but it shows two of the fly bees on the image of FIG. 32A at the left side, wherein it most likely are Cooper-pairs' fly trace. FIG. 32C depicts the peak depth analysis with the results shown the maximum peak depth is 136 nm, and the minimum is 127.4 nm, and peak to peak distance is 8.9 nm.

FIG. 33 depicts the 2D view of the Friedel-oscillation phenomena recorded in the 1.3×1.3 μm² AFM image, that all the elongate-shaped crystalline-like zinc ion atoms oscillating due to the impact from Cooper-pairs electrons moving toward the same direction with tails that indicates the Cooper pairs moving trace. The top toroidal organometallic layer membrane indeed promotes the oscillation compared with the first layer has no superconductivity [28-29]. The AFM images of the second GC/MEA as the cathode MEA assembly with TCD/PEG/PVP/CD copolymer before and after embedded o-nitrophenyl acetate (o-NPA) on the cathode GC electrode was cited in the reference [28-29].

### Example 2

### The Toroidal Josephson Junction Array Model

FIG. 34A depicts the circuitry symbol of the flexible Josephson junction toroidal array electronic quantum Qubit comprising of at least 4 or “n” more junctions and a self-powered switchable reversible DET delocalized electron-relay chain in the center which is the fundamental function of the SAM that the mems-element construction relies on. The FJJT array is a component of a circuit that has the mems-inductor and memristor connected in parallel that connected with a mem-capacitor in a serial position that produced a circuit having multiple-function once the switchable reversible electron-relay current produced an open circuit potential enough to self-powered the chip circuit, herein such as memory storage, operation, and energy storage in the same device without a need of a microwave power supply. The hummingbird's “8” shape fly pattern is a symbolic representation of the intrinsic electron-relay loop within and between the membranes that initiated the cooper pair tunneling and crossed the JJ barriers, and the detailed explanations were in literature [11]. FIG. 34B illustrates an art work representing the matrix of the “Five petal Starflower” layout on the GC electrode surface.

### Example 3

### Engineering Design of an Organo-Metallic Superconductor/MEM-Element Qubit Array Device

FIG. 35 depicts the art model designed of a superconductive/memcapacitive device for energy storage. that comprises the Josephson Junction toroidal array in the GC/S-I-M/GC fabrication. “M” means mem-elements of memristive, memcapacitive, and mem-inductive components. “60” is the GC electrode with a nanopore and pillar membrane; “61” is a symbol indicates the two current collector's connection is reversable. “62” refers to another layer of coating on the membrane embedded with o-nitrophenyl acetate (o-NPA) on top of the GC/MEA; “63” refers to the Pt current collectors; “64” refers to the collagen-1 coating matrix on a dielectric insulator; “65” refers to the circular current flow in a positive direction with the zinc atoms as the brown balls; “66” refers to the cyclodextrin cross-linked copolymers formed “Five-petal Starflower” array matrix alignment with each other produced the eternal superconducting current in the blue circle having induced a Φ₀, single flux quantum, that a non-ferromagnetic field is produced; “67” refers to the Cooper pair. Notice there is an air barrier between the membrane and the array of cyclodextrin matrix. “68” refers to the PEG. . . . PVP's N-terminal chain. The wave of cooper pair electrons is able to long tunnel through the JJ array barriers, even pass through the coated insulator barrier reached to the o-NPA coated GC/MEA on the right side by the Josephson effect 18]. The right-hand side of FIG. 35 depicts the FJJ toroidal array superconducting qubit circuitry comprises memristive, memcapacitive, and meminductive elements powered by the eternal delocalized DET relay current and the mem-inductive current, not relied on the external magnetic field. There are two Pt current collectors alongside the two GC/MEAs, separated by the coated insulator in order to connect with the adaptor cable to the work station. The delocalized DET relay helped to stabilize the d-wave Pi Josephson junction energy at the ground state. The designed Josephson junction toroidal array device has the advantage of multiple-functions as illustrated on the right-side circuitry comprising of mem-elements.

### Example 4

### Collagen Coated on the Insulator as a “Promoter” to Promote Cooper-Pair LJJ Transport

It was a well-known phenomenon that the highest energy of a Pi Josephson junction at the ground state is unstable because its phase shifts to a pi compared with a sinusoidal wave has the lowest Josephson energy at the ground state with a phase equal to zero. FIG. 36A depicts the i-V curves at a scan rate of 10 KHz under different amounts of collagen coating of the insulator compared with the control, in which all device configurations are the same, except the insulator has no collagen coating, under media-free and electrolyte-free conditions. FIG. 36A clearly shows the control has an unstable high energy peak at a ground state of zero-bias compared with the peaks at ±83 mV (±π location) having the lowest energy, but in the presence of a collagen coating insulator at 0.5 ng/mL, it changed the energy unstable states at the ground state to the lowest energy state at zero bias, and all coated situations changed to energy stable at the ground state and ensured the system avoided energy drifting. We noticed that the supercurrent increases more at the lowest collagen concentration compared with other collagen concentrations. FIG. 36B depicts the i-V curves at the scan rate of 20 kHz, the control pi junction peaks at zero bias are at the highest energy because of the pi junction's interference, however at the ground state of zero-bias, in the presence of collagen coating the insulator situation, the pi junction's interference reduced to the minimum, which is energetically favorable for Cooper-pairs long tunneling through the primary and secondary JJ barriers. FIG. 36A and 36B demonstrate collagen acted as a “promoter” that promotes Cooper-pairs tunneling through the LJJ by overcoming the energy instability, indicating a delocalized DET relay formed. and there is no need for a large external magnetic field applied. The i-V curves also provided evidence that the Josephson toroidal junction array device has many slits of junctions, so the up and down nonlinear curves continue, and the abnormal large superconducting peaks occurred in the excited state were observed suggesting the DET relay in the anode double-layer membrane working in a fashion through the Cooper-pairs in a spontaneous way crossed the multiple JJ barriers at zero-bias in the presence of collagen. A similar trend was observed in the half-cells, which do not have a memcapacitive organic membrane-associated cathode MEA as cathode MEA, but the cathode electrode is a platinum wire compared with controls.

The results to access the Cooper-pair LJJ transport with or without the presence of collagen in the dielectric insulator are presented in FIG. 37A for the backward scan at 10 kHz, and 20 kHz compared with controls by the initial rate method using the data obtained from the i-V curves and calculated the ratio of the supercurrent under a given collagen concentration subtract the control's supercurrent at zero-bias then the results divided by the difference of collagen concentration used, so the ratio presents how much supercurrent increase due to per μg/mL collagen at 10 kHz scan rate or 20 kHz scan rate. For example, a 0.5 ng/mL collagen coating caused a supercurrent increase initial rate is 10000 nA/(μg/mL) that further implies the initial rate is 125 μA/(μg/mL)/s according to the scan rate of 10 kHz. FIG. 37B depicts the forward scan results at 10 kHz and 20 kHz respectively comparing the initial rate of collagen concentration between 0.5 ng/mL to 50 μg/mL caused a supercurrent increase over the controls.

The slope of the double-log plot indicates the initial rate increase trend due to collagen having an

inverse relationship with collagen concentration increase, the lowest 0.5 ng/mL collagen has the highest initial rate at 10 kHz, which is 24.209-fold higher than the control. It was noticed higher collagen concentration at 25 to 50 μg/mL reduces more the supercurrent intensity compared with controls at 20 kHz than that at 10 kHz in FIG. 37A as well as in FIG. 37B, which indicate the β_{L} value, which is a characteristic nonlinearity parameter [6, 17]. which equals to 2πLI_{c}/Φ₀, L is the inductance, I_{c} is the critical current, has increased as collagen amount increase at 10 kHz, and the 0.5 ng/mL collagen having the smallest β_{L} value indicating the system's capacitive energy may increase and reduced the magnetic energy impact. FIG. 37C depicts the plots of supercurrent vs. collagen concentrations at a 10 kHz scan rate for a comparison between a whole cell and a half-cell's performance against the controls. The results show there is a 2.54-fold sensitivity increase after coating the insulator for the half-cell compared with the whole cell with 3.15 nA/μg (mL)⁻¹ compared with 1.24 nA/μg (mL)⁻¹ after antilog of the values of the slope, that indicates both linear inductivity from the circular toroidal array and the nonlinear JJ inductivity slowed down, and the charge capacitive energy increased through the Copper pairs promoted delocalized DET, that effectively electrochemical communicated with the function groups between the anode and the cathode MEA crossing the JJ berries. Overall, the whole cell has supercurrent results produced due to the whole cell configuration increased the signal strength by 1.2-fold compared with that of the half-cell, and is the 1.37-fold increase in signal intensity compared with the control of the whole cell. The increase is most likely due to the Copper pair promoting a delocalized DET relay or verse versa. FIG. 37D reveals coating collagen on the insulator with the half-cell device increased the rate of the supercurrent vs. scan of 6.89 nA/m Vs⁻¹ of the half-cell with collagen 0.5 ng/ml to 50 μg/mL compared with 2.1 nA/mV·s⁻¹ of control without an insulator, and 3.6 nA/mV·s⁻¹ of control of the half-cell, which coating increased sensitivity of by 3.23 to 1.92-fold. respectably over 60 Hz and 20 kHz for a half-cell compared with two controls.

### Example 5

### A Typical Gate-Control of Supercurrent by the Five-Petal Starflower Diode Bridge at a Low Scan Rate

A signature gate controlled diode i-V curves with a square peak current crossing zero bias were reported in literature [31-33]. Fabrication methods and validation methods for superconducting nanobridge is critically important for engineering superconducting switches. The gate-controlled supercurrent may lead to superconducting logics like CMOS logics having low energy dissipation. In order to overcome the current technology drawbacks of working at cryogenic conditions and only using metallic related materials, we used the Five-petal Starflower diode bridge as 3D unique Josephson junction array nanostructure, under applied potential in two opposite ways. FIG. 38A and FIG. 38B depict the i-V curves at a lower scan rate of 1 kHz and 300 Hz with and without collagen coating of the insulators, respectively. With 0.5 ng/mL collagen, it shows typical memcapacitor's i-V curves and all other curves are a single square loop peak crossed zero-bias, and the peak intensity was reduced by 2-3-fold from 1 kHz compared with 300 Hz. There was no significant collagen impact observed compared with the controls. Surprisingly there was no unstable pi junction peak observed at the ground state for the controls, which indicates the magnetic inductive energy at high Josephson frequency, for example for our case at 11.16 THz using 50 μμg/mL collagen under scan rate 10 kHz produced 11-fold higher inductive energy than at 0.95 THz with 60 Hz scan rate compared using 150 ng/mL collagen at scan rate 10 kHz with a Josephson frequency 6.7 THz produced only 5.5-fold higher inductive energy than at 60 Hz at 1.2 THz Josephson frequency. FIG. 38A and FIG. 38B imply few JJ arrays' “slits” were actually used at low scan rates than many JJ arrays' “slits” were used shown in FIG. 36A and FIG. 36B.

Observations of suppressing supercurrent down to zero at both directions by change applied potential in forward and backward scans at 1 KHz and 300 Hz, respectively as shown in FIG. 38A, and FIG. 38B for with or without coating of the insulator, indicates the capacitance at 300 Hz has increased to 0.6 nF, 0.98 nF, 0.4 nF, 0.6 nF and 0.63 nF with no collagen. 150 ng/mL, 2.4 μg/mL, 25 μg/mL and 50 μg/mL compared with 1 kHz scan rate. the results are 0.45, 0.38, 0.45, 0.46, and 0.43 nF respectively for the forward scan. Wherein, the potential energy also increased from 12.03, 14.24, 15.5, 18.49 and 10 pj for zero collagen. 150 ng/ml, 2.4 μg/mL, 25 μg/mL and 50 μg/mL collagen at 1 KHz scan rate to 19.2, 30.7, 16.05, 24.32 to 9.71 pj at 300 Hz scan rate for zero collagen, 150 ng/ml, 2.4 μg/mL, 25 μg/mL and 50 μg/mL collagen, respectively.

### Example 6

### The Root of Making LJJ Stable and Flexible at 60 Hz Scan Rate Revealed Between a Full S-I-M Cell and a Half-Cell of S-I-N/Pt

FIG. 38C revealed the i-V curves of the collagen impact at a scan rate of 60 Hz compared with control under the same experimental conditions. The memristive behaviors are shown in the control curve, the 150 ng/ml collagen curve, and 25 μg/mL's i-V curves, whose curves have a hysteresis cross point, for example, the control curve at zero-bias having zero superconductive currents forming a loop “remembering” the past event. Memristors and Memcapacitors are devices made of nanolayers that can mimic neuronal synapses with a characteristic of a hysteresis loop in the i-V curve [19-23]. The i-V curves from the 150 ng/mL collagen look like a memcapacitive curve. Curves with 2.4 μg/mL and 50 μg/mL collagen show the Pi junction behaviors without high energy destructive interference at the ground state at zero bias. A half-cell configuration of GC/S-I-N/Pt was developed for comparison with the whole cell shown in FIG. 39 at a 60 Hz scan rate. There looks like no significant i-V curve difference because most curves have negligible current at zero bias except when collagen concentration is from 2.4 μg/mL up to 50 μg/mL for the half-cell and whole cell. The discovery of a d-wave one-x Josephson junction toroidal array with the whole cell at 2.4 μg/mL collagen changed to a d-wave 4× function with the half-cell configuration under a media-free condition with the same concentration of collagen-coated on the insulator at 60 Hz was our first observation. Our group developed a d-wave 4× Josephson junction toroidal array sensor device with different membrane fabrication having superconductivity on an anode gold chip, and it worked in the PBS solution under 300 Hz scan rate, and without a dielectric insulator and a polymer membrane on our cathode Au electrode [8-9].

We decided to further explore the unknown driver who promotes a delocalized Cooper pair LJJ tunneling through the primary Josephson junction of S-I-M by a method of taking ten consecutive scans of the two devices at a scan rate 60 Hz with no media, to find the supercurrent trend. FIG. 40A depicts the i-V curves of the half-cell of GC/S-I-N/Pt at ten scan cycles with collagen coating concentrations between 0.5 ng/ml to 25 μg/mL compared with the control. FIG. 40B is the i-V curves for a whole cell used for comparison with FIG. 40A. Because the ten consecutive scan curves are overlapping together due to frequent phase change, we cannot immediately obtain an answer. FIG. 41A revealed plots of supercurrent at zero-bias vs. time for 10 cycles scans with different collagen concentrations compared with the control using the half-cell configuration at 60 Hz using the backward scan results. The results show the control device has the lowest phase change rate about 6 times per 10 cycles, compared to 8-9 times per 10 cycles for devices with the collagen coating, and the maximum supercurrent magnitude (from the highest positive supercurrent data to the lowest negative supercurrent data among each of 10 cycle scans) has increased by −15.6%, −22%, +3.9%, +21%, and 0% from 0.5 ng/ml, 150 ng/ml, 600 ng/ml, 2.4 μg/mL, and 25 μg/mL, respectively compared with that of the control. Now we have pieces of evidence that the control has the lowest phase change frequency proportionally to a lower voltage as the second Law of Josephson effect shown in Equation 2 [18].

Results from FIG. 41B using the whole cell of GC/S-I-M/GC configuration show a similar trend in that the control has the lowest phase change frequency per 10 cycles compared with the devices that have collagen coating, and the control signal intensity are same as the control in FIG. 41A. But the maximum supercurrent magnitude increase results are by +42%, −7.1%, +26.2%, and +100% from 0.5 ng/ml, 150 ng/mL, 2.4 μg/mL, and 25 μg/mL, respectively compared with that of the control. These data show the driver who promotes a delocalized Cooper pair LJJ tunneling through the primary and secondary Josephson junction array and magnifies the cell energy is the delocalized DET relay effect coherently reversibly operates the whole system without an external magnetic field applied, wherein this device may be able to act as an energy long-time storage device. Comparison of the performance with the control sensor having the same GC/MEA working assembly, which has no dielectric insulator as shown in FIG. 41C, the i-V curves of the GC/SIS sensor has a supercurrent of 0.46 nA at a state of “1” of the ground-state at zero bias compared the supercurrent −1.7 nA at a state of “−1”, which it seems like a quantum Pauli-Z gate operates on a single qubit state of |1>, and flipped it to −|1>, become a p(π)[M. A. Nielsen and I. Chuang, Quantum Computation and Quantum Information: UV Physics Academy at youtube.com @UVnagee]. There is no positive energy associated with the “−1” qubit bit state in all the i-V curves in the sensor configuration with no dielectric insulator, and no collagen coat, wherein next chapter we will analysis the advantages of using the collagen coating on the insulator.

$$Z \vert 1 >= - \vert 1 >$$

These phenomena observed indicates the d-wave Pi junction in the sensor device energy unstable at the ground state. By contract, FIG. 36A and FIG. 36B for the situations with collagen coating have turned the energy from unstable to stable at the ground state.

### Example 7

### Observations of the DET Relay and the Coating Effect on LJJ Stability at 10 kHz Scan Rate Between a Sensor, a Half-Cell, and a Whole cell in 10 Consecutive Scan Cycles

FIG. 42A depicts the i-V curves of the GC/SIS sensor control in 10 kHz consecutive scan cycles under no media, no dielectric insulator, and no reagent conditions. Comparing the i-V curves in FIG. 42A with the i-V curves in the GC/SIN/Pt half-cell control with a coated insulator in 10 KHz and ten consecutive scan cycles in FIG. 42B, we cannot immediately conclude until the supercurrent data at zero bias at the ground state are extracted and compared shown in FIG. 42E and FIG. 42F. FIG. 42C depicts the i-V curves of the GC/SIN/Pt half-cell at 10 kHz scan rate with collagen coating over 0.5 ng/mL to 50 μμg/mL compared with the control with ten consecutive cycle scans. FIG. 42D depicts the i-V curves of the GC/SIM/GC whole cell at a 10 kHz scan rate with collagen coating over 0.5 ng/mL to 50 μg/mL compared with the control with 10 consecutive cycles scans. The whole cell configuration with coating has higher supercurrent strength compared with the half-cell indicating the DET relay further promoted Cooper-pair LJJ and evidenced by the observations of the Josephson effect crossing the coated insulator reached the cathode GC/MEA assembly without any means of media, reagent, and catalyst at zero bias.

The trend of the supercurrent magnification and the phase change rate among the 10-cycle scans with coating and without coating are assessed in FIG. 42E and FIG. 42F. FIG. 42E revealed the relationship between the supercurrent intensity at zero-bias vs. time among 10 cycles scans with collagen coating compared with control from the half-cell configuration at 10 kHz scan rate at the forward scan from the data obtained in FIG. 42A, FIG. 42B and FIG. 42C with the whole cell configurations shown in FIG. 42F that data obtained from FIG. 42D.

TABLE 1
Performance comparison between the half cell and the whole cell compared
with the control at 10 kHz scan rate among 10 consecutive scan cycles

| | Phase change rate | | Maximum | | Minimum | | | |
|---|---|---|---|---|---|---|---|---|
| | (time among 10 cycle/s) | | supercurrent (nA) | | supercurrent (nA) | | \|Δ(nA)\| | |
| Collagen | Half | Whole | Half | Whole | Half | Whole | Half | Whole |
| (μg/mL) | cell | cell | cell | cell | cell | cell | cell | cell |
| 0 | 2.78 | 2.78 | 6.19 | 6.05 | 0.69 | 0.61 | 5.5 | 5.44 |
| 5E⁻⁴ | 3.12 | 2.78 | 5.13 | 7.37 | 1.54 | −0.03 | 3.59 | 7.4 |
| 0.15 | 3.12 | 2.78 | 4.83 | 6.3 | 1.88 | 0.03 | 2.95 | 6.27 |
| 2.4 | 3.12 | 2.43 | 5.84 | 6.3 | 0.85 | 0.59 | 4.99 | 5.71 |
| 25 | 3.12 | 2.78 | 5.54 | 4.8 | 1.52 | 1.39 | 4.02 | 3.41 |
| 50 | 2.78 | 2.78 | 5.64 | 5.67 | 0.95 | 1.61 | 4.69 | 4.06 |

Results in Table 1 reveal coating collagen among the five levels on the insulator increased the phase change rate at 10 KHz for the half-cell by a 9.8% increase compared with the control, while the whole cell reduced by 2.5% compared with the control. The maximum supercurrent has reduced by 12.8% according to the mean of the five levels compared with the control for the half-cell; the whole cell has an increase by 10% from 0.5 ng/ml to 2.4 μg/mL, but reduced by 13.4% from 25 μg/mL to 50 μg/mL compared with control. The results for the minimum supercurrent are a 95% increase due to collagen coating over the five levels of collagen compared with control for the half-cell; while the whole cell has an increase by 17.7% compared with control over the five levels, a special notice is the minimum supercurrent was reduced by 67.7% between 0.5 ng/ml to 2.4 μg/mL levels compared with the control indicating collagen suppressed on the Josephson effect to focus on the charge energy increase for the energy storage. The higher collagen levels from 25 to 50 g/mL have done damage to our energy storage purpose by increasing the minimum supercurrent by more than 2-fold against the control. Overall, the results of the absolute signal distance between the maximum and the minimum are reduced by 26.4% and 0.7% for the half-cell and the whole cell, respectively over the five collagen levels. In contrast, an increase of 19% of the absolute signal distance over 0.5 ng/mL to 2.4 μg/mL levels compared with the control for the whole cell implies our approach for long energy storage will succeed. Delocalized DET and the coating have promoted Cooper-pair increase the superconductivity and balanced the Josephson nonlinear Eⱼ.

### Example 8

### The JJ Characteristics

The hallmarks of the JJ characteristics are (1) at a DC voltage=0, a supercurrent

$${I}_{s} = {I}_{c} sin ( Δφ ) \qquad ( 1 )$$

I_{c} is critical current, Δφ is the phase difference between the waves of two superconductors, appears at the DC Josephson junction; (2) at a finite DC voltage, the phase of the supercurrent changes as a function of time that caused oscillating at the AC Josephson Junction, which is proportional to 2eV_{DC}, i.e.,

$$\partial φ / \partial t = ( 2 e / h ) {V}_{D C} \qquad ( 2 ) [ 28 ]$$

Stern's group reported the observation of Majorana bound states of Josephson vortices in topological superconductors, and the equations of three types of energy contributions to the Josephson vortices in a long circular junction in a Sine-Gordon system was published [24]. The Josephson junction energy was from the Cooper pair, the magnetic energy was from the inductivity of the circular vortex, and the charge energy was from the SIS quantum capacitor-like device [24]. The vortex suppression of the supercurrent effect also was considered in the equation. The Hamiltonian of the Josephson Junction Array (JJA) was given in the combinations of the first part of charging energy obtained from all arrays and the second part of the Josephson Effect energy [25]. Inspired by their experimental works, our attempt was, by using the 3D dynamic map method, to further seek a method to elucidate the reactions between the component energies to the superconductivity of the vortex array system at room temperature without external magnetic field applied. Our experimental data were shown on the i-V curves and the AFM structure of the superlattice array. The modified Sine-Gordon system energy for our d-wave vortex array is:

$${E}_{JJA}^{n} = ( 1 / 2 ) {{C}_{i}^{- 1} ( Q - e {n}_{1 … i} )}^{2} \qquad ( 3 )$$

$${E}_{L}^{n} = ( 1 / 2 ) {μ}_{0} {N}_{n = 1 ¨ i}^{2} . A . {L}_{n = 1 .. i}^{- 1} . {I}_{n = 1 . . i}^{2} \qquad ( 4 )$$

where Eⁿ_{jjA} is the charge energy of Josephson Junction arrays at n=1 . . . i; Q is the charge, C is the total capacitance at n=1 . . . i, en is the n quantum particles at 1 . . . i data point with an energy periodic in h/e for Josephson effect for d-wave [9]; Eⁿ_{L} is the Inductive energy induced by the circular toroidal array. N is the turning number around the toroidal porous at n=1 . . . i, A is the cross-sectional area of the porous, L is the length of the wending, μ₀ is the magnetic permeability constant in free space; I is current. The toroidal arrays are in series connected. Recent publication regarding our FFTJJ multiple-variable study results in 3D dynamic maps was presented in the literature [9].

Assessing the stability in quantum conductance (Gc) results with or without collagen coating on the insulator was conducted by comparing the results extracted from the i-V curves of 10 consecutive scans at 10 KHz using 150 ng/mL collagen against that of the control. The current data for 10 cycles of forward scans and backward scans, and the corresponding potential used to calculator the quantized Gc. FIG. 42 depicts the 10 cycles scan i-V curves with collagen compared with the control using the whole cell of GC/S-I-M/GC configuration at high scan rate of 10 kHz. FIG. 43 depicts the quantized quantum conductance results for a comparison between the first cycle and the 10th cycle. It is clear evidence that the 150 ng/ml collagen coatings increased quantum conductance over 3 to 7-fold at ±1 mV at 10 kHz scan rate compared with the controls at the 1st, and 10th scan cycles, while the control's quantum conductance values were reduced by 68 to 87.7%.

### Example 9

### Collagen Coating Dielectric Insulator Acted as a Magnifier to Stabilize and Empower the Devices Using a Chronoamperometric Method Under a Zero Voltage Condition

FIG. 44A depicts the plots of peak current at zero voltage vs. phase (Po/It) in a 0.050s per step time interval, a total of 0.10s used in the presence of collagen coating over concentration 0.5 ng/ml to 50 μg/mL on insulators of a half-cell compared with the controls. The typical Josephson AC curves vs. the phase revealed the absolute peak intensity (positive current subtracted from the negative current) has increased by 4.2-fold, 3-fold, 3.3-fold. 2.75-fold, and 2.80-fold for coated collagen 0.5 ng/mL, 600 ng/ml, 2.4 μg/mL, 25 μg/mL, and 50 μg/mL, respectively for the half-cell compared with half-cell control. That Josephson current is inversely proportional to collagen concentration, and they all have a higher total peak intensity than the control's peak. which has the lowest to value having a higher frequency than that of having a coating, indicates the toroidal SQUID inductive of magnetic energy is dominate by the harmonic oscillation at the lower coating than at the higher coating, which Josephson energy increased, which anharmonic oscillating is dominate.

FIG. 44B is for the plots of peak current at zero voltage vs. phase (φ₀/π) under similar conditions as in FIG. 44A but is for the whole-cell in a total 0.10 s in the presence of collagen coating over concentration 2.4 μg/mL to 50 μg/mL compared with the control. Results show the absolute peak current increase due to coating has increased by 27.5% for 2.4 μg/mL coating compared with whole cell control, but for 25 μg/mL and 50 μg/mL concentration, peak intensity reduced by 21.6% and 3.6%, respectively, indicates the phase change caused current increase got control because we want a lasting charge and circular current inductive energy storage device. The observation of coated device overcoming the control's ground state high energy of the d-wave Pi junction to lower energy indicates the system is getting stable.

### Example 10

### Comparing the Stability in Quantum Conductance in 10 Cycle Scans by Multiple Variables Study Using Contour Maps

Results presented in the above section are also presented in contour map forms regarding the quantum conductance relationships with potential and supercurrents with or without 150 ng/ml collagen at 1st and 10th scan cycles in FIG. 45A. 45B for the 1st scan cycle controls in a 3D dynamic map and a 2D contour map, and 45C and 45D are for the control's 10th scan cycle in the dynamic map and the contour map. respectively. We see the contour map's quantum conductance was significantly reduced at the 10th scan cycle for the control device. FIG. 46A. 46B for the 1st scan cycle of the device with 150 ng/ml collagen in a 3D dynamic map and a 2D contour map. respectively. FIG. 46C and FIG. 46D are for the device with 150 ng/ml collagen at the 10th scan cycle in the dynamic map and in the contour map. respectively. For the case with collagen, the maps show the negligible reduction of quantum conductance intensity between the first and the 10th scan cycle. The quantum conductance intensity with collagen, the maps show a 6-8-fold increase compared with the control at the 10th scan cycle, indicates collagen promoted Cooper-pair LJJ tunneling of a long stable quantum conductance, wherein a high energy stability in Ec is expected under the same experimental conditions in time and applied potential for the two cases.

### Example 11

### The LJJ Toroidal Array Device Acted as a Memcapacitive Device Providing Long-Time Stable Energy Storage

The Charge/Discharge Cycles. FIG. 47 depicts the device finished 9000 cycles of charge/discharge at ±30 mA with 50 ms for per cycle, and it reached at a no energy drifting situation under media-free and electrolyte-free conditions at room-temperature.

A Long-time Discharge. After the 9000 cycles charge/discharge, the device with 150 ng/mL collagen coated insulator demonstrates its capability to continue discharge at 50 mA at a nominal voltage 5.4 V for 16.6hours as shown in FIG. 48.

### Example 12

### Further Stability Tests Using the DSCPO Method

FIG. 49A depicts the stability testing using the DSCPO method by comparing a single cycle (50 ms) for charge/discharge at 50 ms at ±30 mA at the first cycle as curve “a” with the collagen 150 ng/mL coated GC/SIM/GC device compared with the control curve “b” without collagen. FIG. 49B depicts the curve “a” for voltage vs. time after 9000 cycles. FIG. 49C depicts the curve “a” after the 9000 cycles in FIG. 49B and after resting for 21 days of testing tested the voltage vs. time. The figures show a robust performance in stability even though the cell voltage kept the same intensity as that of in the beginning after the 9000 cycles and 21 days rest at room temperature.

### Example 13

### Contour Maps Display a Multiple-Variable Study Between the Inductive Energy, Josephson Energy and Collagen Concentrations

The equation of the LJJ array's inductive energy was defined in Section 3.1.1 Equation 4. Results were obtained based on the i-V curves at 10 kHz with different collagen concentrations coated on the insulator shown in FIG. 36A compared with the control. The potential energy stored at the Josephson junction is defined as followings:

$${E}_{p} ( ϕ ) = - {E}_{j} cos ( {ϕ}_{( t )} / φ0 ) \qquad ( 5 ) [ 26 ]$$

$${E}_{J} ≡ {φ}_{0}^{2} / {L}_{j}^{0} \qquad ( 6 ) [ 26 ]$$

$${L}_{j}^{0} ≡ {φ}_{0} / {I}_{0} \qquad ( 7 ) [ 26 ]$$

Eⱼ is called the Josephson energy. Lⱼ⁰ refers to the zero-flux Josephson inductance.
   FIG. 50 shows the highest EL values associated with collagen concentrations of 50 μg/mL, while the Josephson energy Ej is negligible near zero in the contour map plot. It demonstrates when collagen between 0.5 ng/ml to 25 μg/mL has a balanced control of the overwhelming strong (several orders of magnitude) linear magnetic inductance due to the circuitry elements suppressing the nonlinear phase change induced potential energy at the JJ, which confirms our approach of the collagen coating insulator process has a profound impact not only 100% reduced the ground state d-wave pi junction energy unstable, but also suppressed the nonlinear Josephson inductance. FIG. 5I depicts the contour map plot of the capacitive energy of the device as a memcapacitor vs. the nonlinear potential energy stored in the LJJ array and vs. collagen concentrations. The highest memcapacitive energy is located in the region associated with the low collagen concentrations, while the nonlinear potential energy due to phase change induced energy has the most negative energy in −2.5 aj/kg. It further indicates the LJJ array device predominantly serves as a long-time energy storage device.

### Example 14

### Conclusion and Discussions

We demonstrated the benefits of the flexible long Josephson toroidal junction array devices at a solid state without using a media to be able to reach the long-time energy storage goals with stability compared with controls relied on the study of the coating collagen processing onto the insulator systematically, and understanding the effect of the DET relay on the Cooper-pair LJJ. Our new approaches overcome the d-wave pi Josephson junction instability at the ground state, and brought a whole new world of opportunity-a long delocalized LJJ enables DET delocalizing, hence increasing the device utility not only as a quantum computing element but also an energy storage device, and as well as a sensing device. The primary and secondary Josephson junction engineering invasions in the insulator material chosen are appreciative. This technology may help to explore new materials and new membrane fabrication benefiting superconducting energy storage, quantum computing, and quantum sensing purposes.

What is claimed is:

1. A solid-state multiple-functioning D-wave Pi Josephson Junction toroidal array (DPJJTA) quantum interference device (SQUID) comprises of
(a) a first electrode having a first layer of an organic superconductive membrane on top that membrane was made of arrays of nanopillar with cyclodextrin cavities by self-assembly cross-linked copolymers;
(b) a second layer comprising of an organometallic superlattice comprising of a five-petal starflower-like nanostructured membrane that was made of cross-linked triacetyl-β-cyclodextrin (TCD), polyethylene glycol diglycidyl ether (PEG), poly (4-vinylpyridine) (PVP), bis-imidazole derivatized dimethyl-b-cyclodextrin (bM-β-DMCD) and embedded zinc chloride was fabricated by self-assembly horizontally affixed on top of the first layer membrane;
(c) Polymer PEG. . . . TCD and polymer PVP. . . . PEG cross-linked to form polymer chains mimicking a protein choline acetyltransferase (CHAT)'s C-terminal and N-terminal, respectively, and vertically oriented on the surface of the first electrode;
(d) there are Josephson junction barriers comprised of insulators;
(e) a second electrode comprises a double-layer membrane with an electron conductive organic polymer cross-linked formed a poreless flat membrane on top of a first layer nanopore and pillar membrane;
(f) Between two Membrane Electrode Assemblies (MEA) s there is an insulator;
(g) A current collector attached to one of the two MEAs separated by the insulator.

2. A solid-state multiple-functioning D-wave Pi Josephson Junction toroidal array (DPJJTA) SQUID device according to claim 1, wherein uses a scalable 1 cm2 glassy carbon as the electrode.

3. A solid-state multiple-functioning D-wave Pi Josephson Junction toroidal array (DPJJTA) SQUID device according to claim 2, wherein appropriate concentrations of collagen between 0.5 ng/mL to 50 μg/mL are chosen to coat on both sides of a dielectric insulator at 40° C. for two hours for each side.

4. A solid-state multiple-functioning D-wave Pi Josephson Junction toroidal array (DPJJTA) SQUID device according to claim 2, wherein a direct electron-transfer (DET) relay memristive hysteresis i-V current loop formed between the first layer and the second layer membrane on the first electrode, such as biomimetic choline acetyltransferase (CHAT) in the first layer . . . a biomimetic Matrix Metalloproteinase (MMP-2) in the second layer . . . zinc ions being an insulator of the Josephson junction . . . a caped nanopore/-pillar membrane on the second electrode when scanning an electric potential at a low rate of 60 m V/s between ±800mV including passing a zero-bias for the control device with a dielectric insulator without a coating.

5. A solid-state multiple-functioning D-wave Pi Josephson Junction toroidal array (DPJJTA) SQUID device according to claim 4, wherein an anharmonic oscillation phenomenon was observed when scanning an electric potential at a high rate of 10,000 mV/s and up comprising a peak at zero bias with higher energy at a state of “zero” compared with that of an i-V curve fasted a Pauli quantum Z gate operator of Z|1> and transformed to a −|1> state of a P(π) z-direction flip, which the energy at the ground state becomes stable.

6. A solid-state multiple-functioning D-wave Pi Josephson Junction toroidal array (DPJJTA) SQUID device according to claim 1, wherein a superposition qubit “1” state having a high energy and a qubit “0” state with a low energy establishment is to have at least one of the superconductor's membrane with a Friedel-oscillation in the superlattice membrane due to active DET relay, and coating collagen in an appropriate concentration in the dielectric insulator under a media-free operation at room-temperature.

7. A solid-state multiple-functioning D-wave Pi Josephson Junction toroidal array SQUID device according to claim 1, wherein is a solid-state device working under a media-free, electrolyte-free, and catalyst-free at room temperature.

8. A solid-state multiple-functioning D-wave Pi Josephson Junction toroidal array SQUID device according to claim 1, wherein a large “Five-petal Starflowers” structured membrane has a pore depth of 4.56 nm and an outer diameter of 4.8 μm; while a small starflower structure has a star-like pore depth 6.46 nm and a diameter 931 nm with an Rms 14.2 nm among an area 13.7 μm2.

9. A solid-state multiple-functioning D-wave Pi Josephson Junction toroidal array SQUID device according to claim 7, wherein the zinc ion clusters as a “bridge” connecting to the five petals having a barriers' JJ width between 107.7 to 180 nm and a length between 0.89 μm to 1.4 μm with a multiple-layer structure inside of the large cavity of the “Starflower” with a JJ width between 53 nm to 72 nm having a length 180 nm to 360 nm.

10. A solid-state multiple-functioning D-wave Pi Josephson Junction toroidal array SQUID device according to claim 1, wherein the device does not apply an external magnetic field.

11. A solid-state multiple-functioning D-wave Pi Josephson Junction toroidal array SQUID device according to claim 1, wherein comprises mem-elements of memcapacitive, meminductive, and memristive function.

12. A solid-state multiple-functioning D-wave Pi Josephson Junction toroidal array SQUID device according to claim 3, wherein a trend of supercurrent vs. collagen concentrations of coating the insulator was obtained by sensing supercurrent change having a sensitivity 1.24 nA/μg·(mL)⁻¹ vs. 3.15 nA/μg·(mL)⁻¹ for the whole cell over 0.5 ng/ml to 2.4 μg/mL compared to the half-cell over 0.5 ng/ml to 50 μg/mL at 10 kHz forward scan against the controls, respectively.

13. A solid-state multiple-functioning D-wave Pi Josephson Junction toroidal array SQUID device according to claim 3, wherein a trend of supercurrent due to collagen coating compared with controls vs. scan rate over 60 Hz to 20 kHz (5 levels) was obtained by sensing supercurrent change over the scan having a mean sensitivity 6.89 nA/m Vs⁻¹ of the half-cell with collagen 0.5 ng/mL to 50 μg/mL compared with 2.1 nA/m V·s⁻¹ of control without an insulator, and 3.6 nA/mV·s⁻¹ of control of the half-cell, which coating increased sensitivity of by 3.23 to 1.92-fold, respectably.

14. A solid-state multiple-functioning D-wave Pi Josephson Junction toroidal array SQUID device according to claim 3, wherein a trend of initial rate increases due to collagen coating compared with controls vs. collagen concentrations was obtained by sensing an initial rate change for the whole cell at 10 KHz and 20 kHz, respectively with a slope of sensitivity inversely proportional to collagen concentrations of −0.091 nA/μg(mL)⁻¹. At 0.5 ng/ml, the initial rate is 21,348-fold and 24,209-fold higher than the control of 0.53 nA/μg(mL)⁻¹ at 10 kHz forward and backward scan, respectively.

15. A solid-state multiple-functioning D-wave Pi Josephson Junction toroidal array SQUID device according to claim 1, wherein the oscillating wave of the superconductor with insulator coating of 150 ng/ml collagen increased quantum conductance (QC) by 3 and 7-fold at the first scan cycle and 10th cycle of 10KHz compared with controls in a whole cell, while the control without coat, its QC values reduced by 68 and 87.7% at the 10th cycles for forward and backward scan, respectively.

16. A solid-state multiple-functioning D-wave Pi Josephson Junction toroidal array SQUID device according to claim 1, wherein a long-time energy storage property was evidenced by finishing 9000 cycles of charge/discharge at ±30 mA with 50 ms per cycle with 150 ng/ml collagen coating the insulator, and it reached at no energy drifting; it was able to continue discharge at 50 mA at a nominal voltage 5.4 V for 16.6 hours under media-free and electrolyte-free conditions at room temperature.

17. In the use of a solid-state multiple-functioning D-wave Pi Josephson Junction toroidal array SQUID device according to claim 3, a finite supercurrent induced by the electron-relay arises as an eternal energy provider initiating a long-range Josephson junction of toroidal vortices, i.e., intrinsic electromagnetic flux within the JJ boundaries at zero-bias; cooper pairs hop through the junctions causing an anharmonic oscillation due to phase change, herein AC voltage pulses are produced as the qubit's “1”; by the mem-element's reversible loop, when the voltage down to zero with zero current, the qubit's “0” state is granted, furthermore, the quantum state “1” can also be flipped by an embedded Pauli Z-gate due to collagen coating the insulator, to an outcome bit of “−1” state.

18. A solid-state multiple-functioning D-wave Pi Josephson Junction toroidal array SQUID device according to claim 3, wherein provides the function to suppressing supercurrent acting as a gate-controlled diode bridge that increased capacitance and the potential energy storage compared scan rate from 1 kHz and to 300 Hz for with collagen coating compared with the controls. Beyond the square wave region, supercurrent rapidly drop to zero intensity at both directions.
